(19) United States
(12) Patent Application Publication
(10) Pub. No.: US 2026/0335343 A1
(43) Pub. Date: Oct. 29, 2026

(54) COMBINATION THERAPY FOR TREATING OR PREVENTING HIV

(71) Applicant: Gilead Sciences, Inc., Foster City, CA (US)

(72) Inventors: James B.C. Mack, San Mateo, CA (US); Murali Subramanian, Foster City, CA (US); Zaikuan Yu, Foster City, CA (US)

(21) Appl. No.: 19/544,639

(22) Filed: Feb. 19, 2026

Related U.S. Application Data
(60) Provisional application No. 63/761,736, filed on Feb. 21, 2025.

(51) Int. Cl. A61K 31/675; A61K 9/20; A61K 31/41

(52) U.S. Cl. CPC A61K 31/675; A61K 9/2018; A61K 9/2027; A61K 9/2054; A61K 31/41

(57) ABSTRACT

This disclosure relates generally to drug combinations for use in the treatment or prevention of a Retroviridae viral infection, including an infection caused by the human immunodeficiency virus (HIV).

### CROSS REFERENCE TO RELATED APPLICATIONS

This application claims the benefit of U.S. Provisional Application No. 63/761,736, filed Feb. 21, 2025, the entire contents of which is hereby incorporated by reference in its entirety.

### TECHNICAL FIELD

This disclosure relates generally to combinations for use in the treatment or prevention of a Retroviridae viral infection, including an infection caused by the human immunodeficiency virus (HIV). This disclosure also relates to pharmaceutical compositions comprising said combinations.

### BACKGROUND

Positive-single stranded RNA viruses comprising the Retroviridae family include those of the subfamily Orthoretrovirinae and genera Alpharetrovirus, Betaretrovirus, Gammaretrovirus, Deltaretrovirus, Epsilonretrovirus, Lentivirus, and Spumavirus which cause many human and animal diseases. Among the Lentivirus, HIV-1 infection in humans leads to depletion of T helper cells and immune dysfunction, producing immunodeficiency and vulnerability to opportunistic infections. Treating HIV-1 infections with highly active antiretroviral therapies (HAART) has proven to be effective at reducing viral load and significantly delaying disease progression (Hammer, S. M., et al.; JAMA 2008, 300:555-570). However, these treatments could lead to the emergence of HIV strains that are resistant to current therapies (Taiwo, B., International Journal of Infectious Diseases 2009, 13:552-559; Smith, R. J., et al., Science 2010, 327:697-701). Therefore, there is a pressing need to discover new antiretroviral agents that are active against emerging drug-resistant HIV variants.

Also of interest in the area of HIV therapies and treatments is providing regimens to patients with improved pharmacokinetic properties, including, for example, increased potency, long-acting pharmacokinetics, low solubility, low clearance, and/or other properties. While current regimens for treating HIV have progressed enough that patients no longer have to take multiple pills multiple times a day, patients today still are required to take a pill every day for the foreseeable span of their life. Thus, it would be beneficial to have HIV therapies that require patients take medication less than once a day (e.g. once every couple of days, once a week, once every other week, once a month, and so forth) or take a smaller effective dose of the medication(s) on a daily, weekly, monthly, or longer basis.

### SUMMARY

The present application provides, inter alia, a method of treating or preventing a human immunodeficiency virus (HIV) infection in a patient in need thereof, comprising administering to the patient:
(a) a compound of Formula Ia:

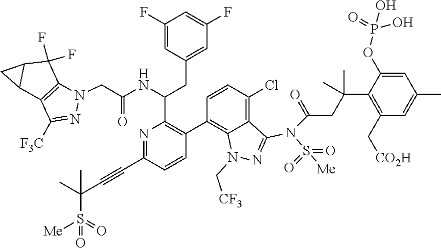 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof; and
(b) a compound of Formula II:

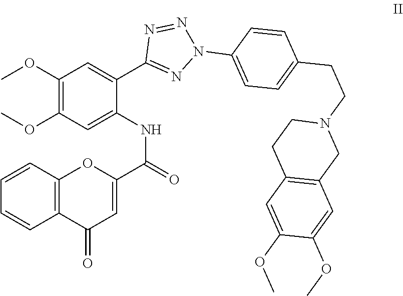 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

or a pharmaceutically acceptable salt thereof.

The present application further provides a method of treating or preventing a human immunodeficiency virus (HIV) infection in a patient in need thereof, comprising administering to the patient a pharmaceutical composition comprising a compound of Formula Ia, or a pharmaceutically acceptable salt thereof, and one or more pharmaceutically acceptable excipients; and a pharmaceutical composition comprising a compound of Formula II, or a pharmaceutically acceptable salt thereof, and one or more pharmaceutically acceptable excipients.

The present application further provides a compound of Formula Ia, or a pharmaceutically acceptable salt thereof, and a compound of Formula II, or a pharmaceutically acceptable salt thereof, for use in any of the methods described herein.

The present application further provides a pharmaceutical composition comprising a compound of Formula Ia, or a pharmaceutically acceptable salt thereof, and one or more pharmaceutically acceptable excipients; and a pharmaceutical composition comprising a compound of Formula II, or a pharmaceutically acceptable salt thereof, and one or more pharmaceutically acceptable excipients, for use in any of the methods described herein.

The present application further provides a compound of Formula Ia, or a pharmaceutically acceptable salt thereof, and a compound of Formula II, or a pharmaceutically acceptable salt thereof, for the preparation of a medicament for use in any of the methods described herein.

The present application further provides a pharmaceutical composition comprising a compound of Formula Ia, or a pharmaceutically acceptable salt thereof, and one or more pharmaceutically acceptable excipients; and a pharmaceutical composition comprising a compound of Formula II, or a pharmaceutically acceptable salt thereof, and one or more pharmaceutically acceptable excipients, for the preparation of a medicament for use in any of the methods described herein.

### DESCRIPTION OF DRAWINGS

FIG. 1 shows lenacapavir % F improvement in male beagle dogs following an oral dose of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4] cyclopenta[1,2-c]pyrazol-1-yl) acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl) acetic acid (i.e., Compound 2) at 55 mg fixed dose with encequidar compared to administration without encequidar.

FIG. 2 shows plasma pharmacokinetic (PK) profiles of encequidar following intravenous (IV) and oral (PO) administration in non-clinical species (monkey, dog, and rat). Plasma concentration-time profiles are shown for IV administration at 1 mg/kg (blue circles) and PO administration at 10 mg/kg (red squares).

FIG. 3 shows pharmacokinetic (PK) profiles of orally administered digoxin (5 mg/kg) and paclitaxel (5 mg/kg) in Sprague-Dawley rats, paclitaxel (5 mg/kg) in beagle dogs, and talinolol (1 mg/kg) in cynomolgus monkeys with and without efflux inhibitors. Encequidar (5 mg/kg, red squares) and elacridar (10 mg/kg, green triangles) were co-administered orally to evaluate their effects on substrate plasma concentrations.

FIG. 4 shows pharmacokinetic (PK) profiles of single-dose intravenous (IV) administration of apixaban (0.33 mg/kg), talinolol (0.33 mg/kg), and lenacapavir (0.33 mg/kg) in beagle dogs, and paclitaxel (1 mg/kg) in Sprague-Dawley rats. Plasma concentration-time curves are shown with) and without co-administration of the P-gp inhibitors (5 mg/kg PO).

### DETAILED DESCRIPTION

The present application provides, inter alia, a method of treating or preventing a human immunodeficiency virus (HIV) infection in a patient in need thereof, comprising administering to the patient an HIV capsid inhibitor in combination with a P-glycoprotein inhibitor (PGP) inhibitor.

In some embodiments, the present disclosure provides a combination of an HIV capsid inhibitor provided herein (e.g., a compound of Formula Ia or Ib, or a pharmaceutically acceptable salt thereof), and a PGP inhibitor (e.g., a compound of Formula II, or a pharmaceutically acceptable salt thereof), or a combination of pharmaceutical compositions each comprising an active agent (i.e., an HIV capsid inhibitor and/or a PGP inhibitor), or a pharmaceutically acceptable salt thereof, provided herein for use in a method of treating or preventing a human immunodeficiency virus (HIV) infection in a patient in need thereof, the method comprising administering to the patient the combination of compounds or compositions provided herein. In some embodiments, the method is a method of treating HIV in a patient. In some embodiments, the method is a method of preventing HIV in a patient.

As used herein, “HIV” or “Human Immunodeficiency Virus” refers to HIV-1 and/or to HIV-2. In some embodiments, the HIV is HIV-1. In some embodiments, the HIV is HIV-2. In some embodiments, the HIV is HIV-1 and HIV-2.

The term “patient” is meant to refer to a human who is in need of therapeutic or preventative treatment for a viral infection, such as HIV infection.

As used herein and in the appended claims, the singular forms “a” and “an”, and “the” include plural referents unless the context clearly dictates otherwise. Thus, e.g., reference to “the compound” includes a plurality of such compounds, “a tablet” or “the tablet” includes reference to one or more tablets, and reference to “the assay” includes reference to one or more assays, and so forth.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, the term “about X” includes description of “X”.

As used herein, the term “consists essentially of” means that, in addition to the mandatory ingredients, specific further ingredients can be present, namely those not materially affecting the essential characteristics of the composition or component in question. Compositions or components herein which “consist essentially of” the specified ingredients may consist only of the specified ingredients, with no other ingredients present.

Kolliphor® HS-15 (CAS No. 70142-34-6) is a non-ionic solubilizer and emulsifying agent obtained by reacting 15 moles of ethylene oxide with 1 mole of 12-hydroxystearic acid. Kolliphor® HS-15 consists of polyglycol mono- and di-esters of 12-hydroxystearic acid (=lipophilic part) and of about 30% of free polyethylene glycol (=hydrophilic part). Kolliphor® HS-15 is also known as Macrogol 15 Hydroxystearate (Ph. Eur.), or Polyoxyl 15 Hydrostearate (USP/NF).

Kolliphor® RH 40 is a non-ionic solubilizer and emulsifying agent obtained by reacting 1 mole of hydrogenated castor oil with 40 moles of ethylene oxide. The main constituent of Kolliphor® RH 40 is glycerol polyethylene glycol hydroxy-stearate, which, together with fatty acid glycerol polyglycol esters, forms the hydrophobic part of the product. The hydrophilic part consists of polyethylene glycols and glycerol ethoxylate. Kolliphor® RH 40 is also known as Macroglycerol Hydroxystearate (Ph. Eur.) or Polyoxyl 40 Hydrogenated Castor Oil (USP/NF). The description provided herein is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading.

In some embodiments, the HIV capsid inhibitor is a compound of Formula Ia:

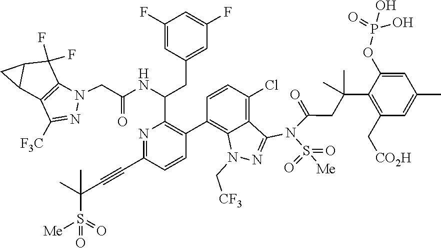 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof.

In some embodiments, the compound of Formula Ia is a compound of Formula Ib:

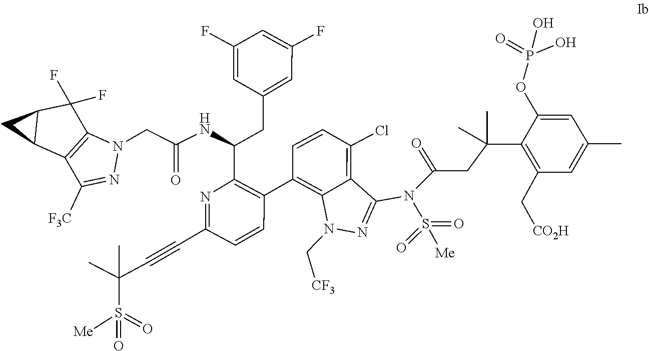 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof. The compound of Formula Ib may also be referred to as Compound 2. Synthesis and characterization of the compounds of Formula Ia and Ib can be found, for example, in U.S. Pat. No. 11,787,825, and U.S. Publication No.: 20240132527, the disclosures of which are incorporated herein by reference in their entireties.

Compound 2 converts to lenacapavir (i.e., N—((S)-1-(3-(4-chloro-3-(methylsulfonamido)-1-(2,2,2-trifluoroethyl)-1H-indazol-7-yl)-6-(3-methyl-3-(methylsulfonyl) but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4] cyclopenta[1,2-c]pyrazol-1-yl) acetamide), an HIV capsid inhibitor that is in development as a long-acting treatment for HIV, in the gastrointestinal tract when administered to a subject (e.g., a human patient).

One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC). Accordingly, the compound structure for Compound 2 provided above may also be named or identified as 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa [3,4] cyclopenta[1,2-c]pyrazol-1-yl) acetamido)-2-(3,5-difluorophenyl)ethyl)-6-(3-methyl-3-(methylsulfonyl) but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl) acetic acid.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds and salts described herein contain one or more asymmetric centers and/or hindered rotation about a bond axis and may thus give rise to enantiomers, diastereomers, and other stereoisomeric forms that may be defined, in terms of absolute stereochemistry, as (R)- or (S)-. The present disclosure is meant to include all such possible isomers, including racemic mixtures, scalemic mixtures, diastereomeric mixtures, optically pure forms and intermediate mixtures. Optically active (R)- and(S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s).

In some embodiments, the compound of Formula Ib is a crystalline form of a compound of Formula Ib (i.e., a crystalline form of Compound 2). In some embodiments, the compound of Formula Ib is crystalline Form I of the compound of Formula Ib (i.e., Compound 2, crystalline Form I).

In some embodiments, the compound of Formula Ib, crystalline Form I has at least one XRPD peak, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least two XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least three XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least four XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least five XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I is characterized by a DSC thermogram having a melting onset at about 202 °C.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. In some embodiments, the crystalline form provided herein may be substantially anhydrous.

Crystalline forms can be identified by solid state characterization methods such as by X-ray powder diffraction (XRPD). Other characterization methods such as differential scanning calorimetry (DSC) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about +3°C depending on the instrument, particular settings, sample preparation, etc. Accordingly, a crystalline form reported herein having a DSC thermogram “about” a particular temperature is understood to accommodate such variation.

In some embodiments, the active ingredient in a composition provided herein is a compound of Formula Ia, or a pharmaceutically acceptable salt thereof. In some embodiments, the active ingredient in a composition provided herein is a compound of Formula Ia.

In some embodiments, the active ingredient in a composition provided herein is a compound of Formula Ib, or a pharmaceutically acceptable salt thereof. In some embodiments, the active ingredient in a composition provided herein is a compound of Formula Ib.

In some embodiments, the P-glycoprotein (PGP) inhibitor provided herein is selected from verapamil, dexverapamil, cyclosporine, zosuquidar, laniquidar, elacridar, tariquidar, and encequidar.

In some embodiments, the PGP inhibitor is a compound of Formula II:

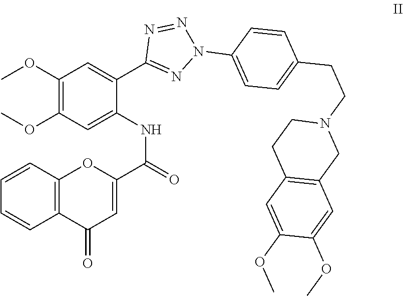 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

or a pharmaceutically acceptable salt thereof. The compound of Formula II may also be referred to as encequidar (or ENC) or N-(2-(2-(4-(2-(6,7-dimethoxy-3,4-dihydroisoquinolin-2 (1H)-yl)ethyl) phenyl)-2H-tetrazol-5-yl)-4,5-dimethoxyphenyl)-4-oxo-4H-chromene-2-carboxamide. In some embodiments, the PGP inhibitor is a pharmaceutically acceptable salt of encequidar. In some embodiments, the PGP inhibitor is a mesylate salt of encequidar.

Increased intestinal expression of P-glycoprotein can reduce the absorption of drugs that are substrates for P-glycoprotein. PGP inhibitors may therefore be useful to modulate the PK properties of HIV capsid inhibitors (e.g., compounds of Formula Ia or Ib) disclosed herein. In some embodiments, the use of a PGP inhibitor in combination with a compound of Formula Ia or Ib, or a pharmaceutically acceptable salt thereof, disclosed herein can modulate (e.g. increase) the PK properties of the compound of Formula I, such as AUC, Cₘₐₓ, and oral bioavailability.

In some embodiments, the methods provided herein comprise administering to the subject about 1 mg to 4,000 mg of the PGP inhibitor, for example, about 2,000 to 4,000 mg, about 1 to 2,000 mg, about 1 to 1,000 mg, about 1 to 500 mg, about 10 to 500 mg, about 20 to 500 mg, about 1 to 300 mg, about 10 to 300 mg, about 20 to 300 mg, about 1 to 200 mg, about 10 to 200 mg, about 20 to 200 mg, about 1 to 100 mg, about 10 to 100 mg, about 20 to 100 mg, about 50 to 300 mg, about 75 to 200 mg, or about 15 to 150 mg of the PGP inhibitor.

In some embodiments, the methods provided herein comprise administering to the subject about 1 mg to about 50 mg of the PGP inhibitor.

In some embodiments, the methods provided herein comprise administering to the subject about 10 mg to about 50 mg of the PGP inhibitor.

In some embodiments, the methods provided herein comprise administering to the subject about 10 mg to about 30 mg of the PGP inhibitor.

In some embodiments, the methods provided herein comprise administering to the subject about 10 mg to about 20 mg of the PGP inhibitor.

In some embodiments, the methods provided herein comprise administering to the subject about 14 mg to about 16 mg of the PGP inhibitor.

In some embodiments, the methods provided herein comprise administering to the subject about 15 mg of the PGP inhibitor.

In some embodiments, the methods provided herein comprise administering to the subject about 1 mg to 4,000 mg of the compound of Formula II, or a pharmaceutically acceptable salt thereof, for example, about 2,000 to 4,000 mg, about 1 to 2,000 mg, about 1 to 1,000 mg, about 1 to 500 mg, about 10 to 500 mg, about 20 to 500 mg, about 1 to 300 mg, about 10 to 300 mg, about 20 to 300 mg, about 1 to 200 mg, about 10 to 200 mg, about 20 to 200 mg, about 1 to 100 mg, about 10 to 100 mg, about 20 to 100 mg, about 50 to 300 mg, about 75 to 200 mg, or about 15 to 150 mg of the compound of Formula II, or a pharmaceutically acceptable salt thereof.

In some embodiments, the methods provided herein comprise administering to the subject about 1 mg to about 50 mg of the compound of Formula II, or a pharmaceutically acceptable salt thereof. In some embodiments, the methods provided herein comprise administering to the subject about 10 mg to about 50 mg of the compound of Formula II, or a pharmaceutically acceptable salt thereof. In some embodiments, the methods provided herein comprise administering to the subject about 10 mg to about 30 mg of the compound of Formula II, or a pharmaceutically acceptable salt thereof. In some embodiments, the methods provided herein comprise administering to the subject about 10 mg to about 25 mg of the compound of Formula II, or a pharmaceutically acceptable salt thereof. In some embodiments, the methods provided herein comprise administering to the subject about 15 mg to about 25 mg of the compound of Formula II, or a pharmaceutically acceptable salt thereof. In some embodiments, the methods provided herein comprise administering to the subject about 15 mg to about 20 mg of the compound of Formula II, or a pharmaceutically acceptable salt thereof. In some embodiments, the methods provided herein comprise administering to the subject about 10 mg to about 20 mg of the compound of Formula II, or a pharmaceutically acceptable salt thereof. In some embodiments, the methods provided herein comprise administering to the subject about 19 mg to about 21 mg of the compound of Formula II, or a pharmaceutically acceptable salt thereof. In some embodiments, the methods provided herein comprise administering to the subject about 20 mg of the compound of Formula II, or a pharmaceutically acceptable salt thereof. In some embodiments, the methods provided herein comprise administering to the subject about 14 mg to about 16 mg of the compound of Formula II, or a pharmaceutically acceptable salt thereof. In some embodiments, the methods provided herein comprise administering to the subject about 15 mg of the compound of Formula II, or a pharmaceutically acceptable salt thereof.

In some embodiments, the methods provided herein comprise administering to the subject about 1 mg to about 50 mg of a pharmaceutically acceptable salt of the compound of Formula II. In some embodiments, the methods provided herein comprise administering to the subject about 10 mg to about 50 mg of a pharmaceutically acceptable salt of the compound of Formula II. In some embodiments, the methods provided herein comprise administering to the subject about 10 mg to about 30 mg of a pharmaceutically acceptable salt of the compound of Formula II. In some embodiments, the methods provided herein comprise administering to the subject about 10 mg to about 25 mg of a pharmaceutically acceptable salt of the compound of Formula II. In some embodiments, the methods provided herein comprise administering to the subject about 15 mg to about 25 mg of a pharmaceutically acceptable salt of the compound of Formula II. In some embodiments, the methods provided herein comprise administering to the subject about 15 mg to about 20 mg of a pharmaceutically acceptable salt of the compound of Formula II. In some embodiments, the methods provided herein comprise administering to the subject about 10 mg to about 20 mg of a pharmaceutically acceptable salt of the compound of Formula II. In some embodiments, the methods provided herein comprise administering to the subject about 19 mg to about 21 mg of a pharmaceutically acceptable salt of the compound of Formula II. In some embodiments, the methods provided herein comprise administering to the subject about 20 mg of a pharmaceutically acceptable salt of the compound of Formula II. In some embodiments, the methods provided herein comprise administering to the subject about 14 mg to about 16 mg of a pharmaceutically acceptable salt of the compound of Formula II. In some embodiments, the methods provided herein comprise administering to the subject about 15 mg of a pharmaceutically acceptable salt of the compound of Formula II.

In some embodiments, the methods provided herein comprise administering to the subject about 1 mg to about 50 mg of a mesylate salt of the compound of Formula II. In some embodiments, the methods provided herein comprise administering to the subject about 10 mg to about 50 mg of a mesylate salt of the compound of Formula II. In some embodiments, the methods provided herein comprise administering to the subject about 10 mg to about 30 mg of a mesylate salt of the compound of Formula II. In some embodiments, the methods provided herein comprise administering to the subject about 10 mg to about 25 mg of a mesylate salt of the compound of Formula II. In some embodiments, the methods provided herein comprise administering to the subject about 15 mg to about 25 mg of a mesylate salt of the compound of Formula II. In some embodiments, the methods provided herein comprise administering to the subject about 15 mg to about 20 mg of a mesylate salt of the compound of Formula II. In some embodiments, the methods provided herein comprise administering to the subject about 10 mg to about 20 mg of a mesylate salt of the compound of Formula II. In some embodiments, the methods provided herein comprise administering to the subject about 19 mg to about 21 mg of a mesylate salt of the compound of Formula II. In some embodiments, the methods provided herein comprise administering to the subject about 20 mg of a mesylate salt of the compound of Formula II. In some embodiments, the methods provided herein comprise administering to the subject about 14 mg to about 16 mg of a mesylate salt of the compound of Formula II. In some embodiments, the methods provided herein comprise administering to the subject about 15 mg of a mesylate salt of the compound of Formula II.

The methods provided herein comprise administration of compounds provided herein (e.g., administration of a compound of Formula Ia or Ib, and a compound of Formula II), and salts thereof, such as pharmaceutically acceptable salts. A salt generally refers to a derivative of a disclosed compound wherein the parent compound is modified by converting an existing acid or base moiety to its salt form. A pharmaceutically acceptable salt is one that, within the scope of sound medical judgment, is suitable for use in contact with the tissues of human beings and animals without excessive toxicity, irritation, allergic response, or other problem or complication, commensurate with a reasonable benefit/risk ratio. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

### Methods of Treating or Preventing HIV

In some embodiments, the present disclosure provides a method of treating or preventing a human immunodeficiency virus (HIV) infection in a patient in need thereof, comprising administering to the patient:
(a) a compound of Formula Ia:

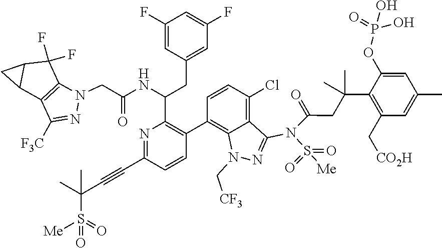 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof; and
(b) a compound of Formula II:

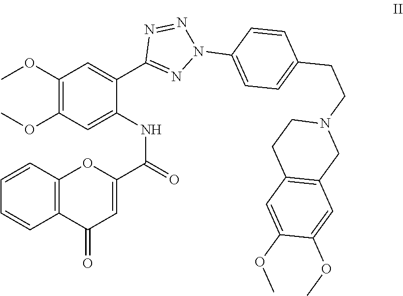 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

or a pharmaceutically acceptable salt thereof.

In some embodiments, the method provided herein is a method of treating a human immunodeficiency virus (HIV) infection in a patient. In some embodiments, the method comprises administering to the patient a therapeutically effective amount of the compounds (e.g., a compound of Formula Ia or Ib, or a pharmaceutically acceptable salt thereof, and a compound of Formula II, or a pharmaceutically acceptable salt thereof) or compositions provided herein.

The term “therapeutically effective amount” refers to an amount of the compounds or compositions provided herein, which when administered to a patient in need thereof, is sufficient (e.g., sufficient in combination) to effect treating an HIV infection, as described herein. Such an amount would be sufficient to elicit the biological or medical response of a tissue system, or patient that is sought by a researcher or clinician. The amount of the compounds which constitute therapeutically effective amounts will vary depending on such factors as the compound, salt, or composition used for administration, the time of administration, the route of administration, the rate of excretion of the compound, the duration of the treatment, the type of disease-state or disorder being treated and its severity, drugs used in combination with or coincidentally with the compounds provided herein, and the age, body weight, general health, sex and diet of the patient. Such therapeutically effective amounts can be determined routinely by one of ordinary skill in the art having regard to their own knowledge, the state of the art, and this disclosure.

In some embodiments, the method provided herein is a method of treating a human immunodeficiency virus (HIV) infection in a heavily treatment-experienced patient.

As used herein, a “heavily treatment-experienced patient” refers to an HIV-infected patient who has limited treatment options due to a multidrug resistant HIV infection. For example, in some embodiments, the “heavily treatment-experienced patient” is a patient with HIV who has developed resistance to an antiretroviral medication from at least one class of antiretroviral medications selected from the group consisting of NRTIs, NNRTIs, PIs, and INSTIs.

In some embodiments, a patient is a heavily treatment-experienced patient with multidrug resistant HIV-1.

In some embodiments, “multidrug resistant HIV infection” means resistance to an antiretroviral medication from at least one class of antiretroviral medications selected from the group consisting of NRTIs, NNRTIs, PIs, and INSTIs. In some embodiments, “multidrug resistant HIV infection” means resistance to at least one antiretroviral medication from two classes of antiretroviral medications selected from the group consisting of NRTIs, NNRTIs, PIs, and INSTIs. In some embodiments, “multidrug resistant HIV infection” means resistance to at least one antiretroviral medication from three classes of antiretroviral medications selected from the group consisting of NRTIs, NNRTIs, PIs, and INSTIs. In some embodiments, “multidrug resistant HIV infection” means resistance to at least one antiretroviral medication from each of the four classes of antiretroviral medications selected from the group consisting of NRTIs, NNRTIs, PIs, and INSTIs.

As used herein, the term “NRTI(s)” refers to nucleoside reverse transcriptase inhibitor(s) or nucleotide reverse transcriptase inhibitor(s).

As used herein, the term “NNRTI(s)” refers to non-nucleoside reverse transcriptase inhibitor(s) or non-nucleotide reverse transcriptase inhibitor(s).

As used herein, the term “PI(s)” refers to protease inhibitor(s).

As used herein, the term “INSTI(s)” refers to integrase strand transfer inhibitor(s). As used herein, the term “fail” or “failed” when referring to HIV therapy or an HIV treatment regimen means a treatment outcome which precludes the use of the same agent or class in the future in a patient with HIV. This could be due to inadequate initial viral response due to pre-existing viral resistance, viral rebound due to emergent viral resistance, or inability of a patient to continue a treatment due to intolerability or safety issues.

In the disclosed methods, the heavily treatment-experienced patient is infected with multidrug resistant HIV. In some embodiments, the heavily treatment-experienced patient has a multidrug resistant HIV infection and is on a failing HIV treatment regimen. In some embodiments, the heavily treatment-experienced patient has a viral load greater than about 1,000 copies of HIV RNA/mL.

In some embodiments, the HIV infection is an HIV-1 infection. In some embodiments, the HIV-1 infection is characterized by HIV-1 mutant resistance to an antiretroviral medication, for example, to one, two, three, four, or more classes of antiretroviral medications (e.g., PIs, NRTIs, NNRTIs, INSTIs, etc.). In some embodiments, the HIV-1 infection is characterized by HIV-1 mutant resistance to one or more classes of antiretroviral medications. In some embodiments, the HIV-1 infection is characterized by HIV-1 mutant resistance to two or more classes of antiretroviral medications. In some embodiments, the HIV-1 infection is characterized by HIV-1 mutant resistance to three or more classes of antiretroviral medications.

In some embodiments, the HIV-1 infection is characterized by an HIV-1 mutant that includes, but is not limited to:
(a) an HIV-1 mutant resistant to a PI (e.g., 150V, 184V/L90M, G48V/V82A/L90M, G48V/V82S, etc.);
(b) an HIV-1 mutant resistant to an NRTI (e.g., K65R, M184V, 6TAMs, etc.);
(c) an HIV-1 mutant resistant to an NNRTI (e.g., K103N, Y181C, Y188L, L100I/K103N, K103N/Y181C, etc.); and/or
(d) an HIV-1 mutant resistant to an INSTI (Y143R, E138K/Q148K, G140S/Q148R, E92Q/N155H, N155H/Q148R, R263K/M50I, etc.).

In some embodiments, the patient is infected with HIV-1 that is resistant to at least one antiretroviral medication. In some embodiments, the patient is infected with multidrug resistant HIV-1. In some embodiments, the patient is infected with multidrug resistant HIV-1 that is resistant to at least one, two, three, four, or more antiretroviral medications. In some embodiments, the patient is infected with multidrug resistant HIV-1 that is resistant to at least one antiretroviral medication from each of two different classes of antiretroviral medications. In some embodiments, the patient is infected with multidrug resistant HIV-1 that is resistant to at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI). In some embodiments, the different classes of antiretroviral medications are selected from an NRTI, an NNRTI, and a PI. In some embodiments, the patient is infected with multidrug resistant HIV-1 that is resistant to at least one NRTI and at least one NNRTI. In some embodiments, the patient is infected with multidrug resistant HIV-1 that is resistant to at least one NRTI and at least one PI. In some embodiments, the patient is infected with multidrug resistant HIV-1 that is resistant to at least one NRTI and at least one INSTI. In some embodiments, the patient is infected with multidrug resistant HIV-1 that is resistant to at least one NNRTI and at least one PI. In some embodiments, the patient is infected with multidrug resistant HIV-1 that is resistant to at least one NNRTI and at least one INSTI. In some embodiments, the patient is infected with multidrug resistant HIV-1 that is resistant to at least one PI and at least one INSTI. In some embodiments, the patient is infected with multidrug resistant HIV-1 that is resistant to at least one NRTI, at least one NNRTI, and at least one PI. In some embodiments, the patient is infected with multidrug resistant HIV-1 that is resistant to at least one NRTI, at least one NNRTI, and at least one INSTI. In some embodiments, the patient is infected with multidrug resistant HIV-1 that is resistant to at least one NRTI, at least one PI, and at least one INSTI. In some embodiments, the patient is infected with multidrug resistant HIV-1 that is resistant to at least one NNRTI, at least one PI, and at least one INSTI. In some embodiments, the patient is infected with multidrug resistant HIV-1 that is resistant to at least one NRTI, at least one NNRTI, at least one PI, and at least one INSTI.

In some embodiments, the patient is infected with multidrug resistant HIV-1 that is resistant to at least one antiretroviral medication that is an NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®)), stavudine (d4T; Zerit®), zalcitabine (dideoxycytidine, ddC; Hivid®), and abacavir (Ziagen®).

In some embodiments, the patient is infected with multidrug resistant HIV-1 that is resistant to at least one antiretroviral medication that is an NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the patient is infected with multidrug resistant HIV-1 that is resistant to at least one antiretroviral medication that is a PI. Examples of PIs include, but are not limited to, amprenavir (Agenerase®), atazanavir (Reyataz®), darunavir (Prezista®), fosamprenavir (Telzir®), Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the patient is infected with multidrug resistant HIV-1 that is resistant to at least one antiretroviral medication that is an INSTI. Examples of INSTIs include, but are not limited to, raltegravir (Isentress®), elvitegravir (Vitekta®), dolutegravir (Tivicay®), cabotegravir, and bictegravir.

In some embodiments, the patient is infected with multidrug resistant HIV-1 that is resistant to at least one antiretroviral medication that is a gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In some embodiments, the patient is infected with multidrug resistant HIV-1 that is resistant to at least one antiretroviral medication that is a CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

In some embodiments of the disclosed methods, the patient has been previously treated with at least one antiretroviral medication before being treated with the compounds (e.g., a compound of Formula Ia or Ib, or a pharmaceutically acceptable salt thereof, and a compound of Formula II, or a pharmaceutically acceptable salt thereof) or compositions provided herein. In some embodiments, the patient has been previously treated with at least one antiretroviral medication for at least 3 months, such as at least 4 months, at least 5 months, at least 6 months, at least 7 months, at least 8 months, at least 9 months, at least 10 months, at least 11 months, at least 12 months, at least 18 months, or at least 24 months. In some embodiments, the patient has been previously treated with at least one antiretroviral medication for at least 3 months. In some embodiments, the patient has been previously treated with at least one antiretroviral medication for at least 6 months. In some embodiments, the patient has been previously treated with at least one antiretroviral medication for at least 9 months. In some embodiments, the patient has been previously treated with at least one antiretroviral medication for at least 12 months. In some embodiments, the patient has been previously treated with at least one antiretroviral medication for at least 18 months. In some embodiments, the patient has been previously treated with at least one antiretroviral medication for at least 24 months. In some embodiments, the patient has been previously treated with at least one antiretroviral medication for at least 30 months. In some embodiments, the patient has been previously treated with at least one antiretroviral medication for at least 36 months.

In some embodiments of the disclosed methods, the patient has failed or is failing an HIV treatment regimen before being treated with the compounds or compositions provided herein. In some embodiments, the prior HIV treatment regimen included administering at least one antiretroviral medication. In some embodiments, the patient infected with HIV has relapsed after an initial response to the prior HIV treatment regimen, for example, antiretroviral therapy. In some embodiments, the patient has a viral load of greater than about 50 copies of HIV RNA/mL after about 48 weeks of therapy, for example, antiretroviral therapy, before being treated with the compounds or compositions provided herein.

In some embodiments, the prior treatment regimen includes administering at least one antiretroviral medication from each of two different classes of antiretroviral medications. In some embodiments, the prior treatment regimen includes administering at least one antiretroviral medication from each of three different classes of antiretroviral medications. In some embodiments, the different classes of antiretroviral medications are selected from a nucleoside reverse transcriptase inhibitor (NRTI), a non-nucleoside reverse transcriptase inhibitor (NNRTI), a protease inhibitor (PI), and an integrase strand transfer inhibitor (INSTI). In some embodiments, the different classes of antiretroviral medications are selected from an NRTI, an NNRTI, and a PI. In some embodiments, the prior treatment regimen includes administering at least one NRTI and at least one NNRTI. In some embodiments, the prior treatment regimen includes administering at least one NRTI and at least one PI. In some embodiments, the prior treatment regimen includes administering at least one NRTI and at least one INSTI. In some embodiments, the prior treatment regimen includes administering at least one NNRTI and at least one PI. In some embodiments, the prior treatment regimen includes administering at least one NNRTI and at least one INSTI. In some embodiments, the prior treatment regimen includes administering at least one PI and at least one INSTI. In some embodiments, the prior treatment regimen includes administering at least one NRTI, at least one NNRTI, and at least one PI. In some embodiments, the prior treatment regimen includes administering at least one NRTI, at least one NNRTI, and at least one INSTI. In some embodiments, the prior treatment regimen includes administering at least one NRTI, at least one PI, and at least one INSTI. In some embodiments, the prior treatment regimen includes administering at least one NNRTI, at least one PI, and at least one INSTI.

In some embodiments, the prior treatment regimen includes administering at least one antiretroviral medication that is a gp41 fusion inhibitor.

In some embodiments, the prior treatment regimen includes administering at least one antiretroviral medication that is a CCR5 co-receptor antagonist.

In some embodiments, the prior treatment regimen includes administering at least one antiretroviral medication that is an NRTI. Examples of NRTIs include, but are not limited to, emtricitabine (FTC; Emtriva®), lamivudine (3TC; Epivir®), zidovudine (azidothymidine (AZT); Retrovir®), didanosine (ddI; Videx-EC®), dideoxyinosine (Videx®), tenofovir, tenofovir alafenamide (Vemlidy®), tenofovir disoproxil fumarate (Viread®), stavudine (d4T; Zerit®), zalcitabine (dideoxycytidine, ddC; Hivid®), and abacavir (Ziagen®).

In some embodiments, the prior treatment regimen includes administering at least one antiretroviral medication that is an NNRTI. Examples of NNRTIs include, but are not limited to, efavirenz (Sustiva®), etravirine (Intelence®), rilpivirine (Edurant®), nevirapine (Viramune®), and delavirdine (Rescriptor®).

In some embodiments, the prior treatment regimen includes administering at least one antiretroviral medication that is a PI. Examples of PIs include, but are not limited to, amprenavir (Agenerase®), atazanavir (Reyataz®), darunavir (Prezista®), fosamprenavir (Telzir®), Lexiva®), indinavir (Crixivan®), lopinavir (Kaletra®), nelfinavir (Viracept®), ritonavir (Norvir®), saquinavir (Invirase®), and tipranavir (Aptivus®).

In some embodiments, the prior treatment regimen includes administering at least one antiretroviral medication that is an INSTI. Examples of INSTIs include, but are not limited to, raltegravir (Isentress®), elvitegravir (Vitekta®), dolutegravir (Tivicay®), cabotegravir, and bictegravir.

In some embodiments, the prior treatment regimen includes administering at least one antiretroviral medication that is a gp41 fusion inhibitor. Examples of gp41 fusion inhibitors include, but are not limited to, albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

In some embodiments, the prior treatment regimen includes administering at least one antiretroviral medication that is a CCR5 co-receptor antagonist. Examples of CCR5 co-receptor antagonists include, but are not limited to, aplaviroc, vicriviroc, maraviroc, cenicriviroc, PRO-140, adaptavir (RAP-101), nifeviroc (TD-0232), anti-GP120/CD4 or CCR5 bispecific antibodies, B-07, MB-66, polypeptide C25P, TD-0680, and vMIP (Haimipu).

In some embodiments of the disclosed methods, the heavily treatment-experienced patient infected with HIV has a viral load of about 200 copies of HIV-1 RNA/mL (c/mL) to about 1,000,000 c/mL at the time of beginning administration of the compounds or compositions provided herein, such as a viral load of about 200 c/mL to about 500,000 c/mL, about 200 c/mL to about 250,000 c/mL, about 200 c/mL to about 100,000 c/mL, about 200 c/mL to about 50,000 c/mL, about 200 c/mL to about 25,000 c/mL, about 200 c/mL to about 10,000 c/mL, about 200 c/mL to about 5,000 c/mL, about 200 c/mL to about 3,000 c/mL, about 200 c/mL to about 2,000 c/mL, about 200 c/mL to about 1,000 c/mL, about 200 c/mL to about 750 c/mL, about 200 c/mL to about 500 c/mL, about 500 c/mL to about 1,000,000 c/mL, about 500 c/mL to about 500,000 c/mL, about 500 c/mL to about 250,000 c/mL, about 500 c/mL to about 100,000 c/mL, about 500 c/mL to about 50,000 c/mL, about 500 c/mL to about 25,000 c/mL, about 500 c/mL to about 10,000 c/mL, about 500 c/mL to about 5,000 c/mL, about 500 c/mL to about 3,000 c/mL, about 500 c/mL to about 2,000 c/mL, about 500 c/mL to about 1,000 c/mL, about 500 c/mL to about 750 c/mL, about 750 c/mL to about 1,000,000 c/mL, about 750 c/mL to about 500,000 c/mL, about 750 c/mL to about 250,000 c/mL, about 750 c/mL to about 100,000 c/mL, about 750 c/mL to about 50,000 c/mL, about 750 c/mL to about 25,000 c/mL, about 750 c/mL to about 10,000 c/mL, about 750 c/mL to about 5,000 c/mL, about 750 c/mL to about 3,000 c/mL, about 750 c/mL to about 2,000 c/mL, about 750 c/mL to about 1,000 c/mL, about 1,000 c/mL to about 1,000,000 c/mL, about 1,000 c/mL to about 500,000 c/mL, about 1,000 c/mL to about 250,000 c/mL, about 1,000 c/mL to about 100,000 c/mL, about 1,000 c/mL to about 50,000 c/mL, about 1,000 c/mL to about 25,000 c/mL, about 1,000 c/mL to about 10,000 c/mL, about 1,000 c/mL to about 5,000 c/mL, about 1,000 c/mL to about 3,000 c/mL, about 1,000 c/mL to about 2,000 c/mL, about 2,000 c/mL to about 1,000,000 c/mL, about 2,000 c/mL to about 500,000 c/mL, about 2,000 c/mL to about 250,000 c/mL, about 2,000 c/mL to about 100,000 c/mL, about 2,000 c/mL to about 50,000 c/mL, about 2,000 c/mL to about 25,000 c/mL, about 2,000 c/mL to about 10,000 c/mL, about 2,000 c/mL to about 5,000 c/mL, about 2,000 c/mL to about 3,000 c/mL, about 3,000 c/mL to about 1,000,000 c/mL, about 3,000 c/mL to about 500,000 c/mL, about 3,000 c/mL to about 250,000 c/mL, about 3,000 c/mL to about 100,000 c/mL, about 3,000 c/mL to about 50,000 c/mL, about 3,000 c/mL to about 25,000 c/mL, about 3,000 c/mL to about 10,000 c/mL, about 3,000 c/mL to about 5,000 c/mL, about 5,000 c/mL to about 1,000,000 c/mL, about 5,000 c/mL to about 500,000 c/mL, about 5,000 c/mL to about 250,000 c/mL, about 5,000 c/mL to about 100,000 c/mL, about 5,000 c/mL to about 50,000 c/mL, about 5,000 c/mL to about 25,000 c/mL, about 5,000 c/mL to about 10,000 c/mL, about 10,000 c/mL to about 1,000,000 c/mL, about 10,000 c/mL to about 500,000 c/mL, about 10,000 c/mL to about 250,000 c/mL, about 10,000 c/mL to about 100,000 c/mL, about 10,000 c/mL to about 50,000 c/mL, about 10,000 c/mL to about 25,000 c/mL, about 25,000 c/mL to about 1,000,000 c/mL, about 25,000 c/mL to about 500,000 c/mL, about 25,000 c/mL to about 250,000 c/mL, about 25,000 c/mL to about 100,000 c/mL, about 25,000 c/mL to about 50,000 c/mL, about 50,000 c/mL to about 1,000,000 c/mL, about 50,000 c/mL to about 500,000 c/mL, about 50,000 c/mL to about 250,000 c/mL, about 50,000 c/mL to about 100,000 c/mL, about 100,000 c/mL to about 1,000,000 c/mL, about 100,000 c/mL to about 500,000 c/mL, about 100,000 c/mL to about 250,000 c/mL, about 250,000 c/mL to about 1,000,000 c/mL, about 250,000 c/mL to about 500,000 c/mL, or about 500,000 c/mL to about 1,000,000 c/mL.

In some embodiments, the patient has a viral load of greater than about 200 copies of HIV-1 RNA/mL (c/mL) at the time of beginning administration of the compounds or compositions provided herein, such as a viral load greater than about 500 c/mL, about 750 c/mL, about 1,000 c/mL, about 2,000 c/mL, about 3,000 c/mL, about 5,000 c/mL, about 10,000 c/mL, about 25,000 c/mL, about 50,000 c/mL, about 100,000 c/mL, about 250,000 c/mL, about 500,000 c/mL, or greater than about 1,000,000 c/mL at the time of beginning administration of the compounds or compositions provided herein. In some embodiments, the patient has a viral load of greater than about 200 c/mL at the time of beginning administration of the compounds or compositions provided herein. In some embodiments, the patient has a viral load of greater than about 500 c/mL at the time of beginning administration of the compounds or compositions provided herein. In some embodiments, the patient has a viral load of greater than about 750 c/mL at the time of beginning administration of the compounds or compositions provided herein. In some embodiments, the patient has a viral load of greater than about 1,000 c/mL at the time of beginning administration of the compounds or compositions provided herein. In some embodiments, the patient has a viral load of greater than about 2,000 c/mL at the time of beginning administration of the compounds or compositions provided herein.

In some embodiments of the disclosed methods, administration of the compounds or compositions provided herein, results in a decrease in the viral load in the patient. In some embodiments, the viral load is decreased by about 0.5 log 10 to about 2.5 log 10 after administration of the compounds or compositions provided herein, for a certain amount of time as compared to the viral load at the time of beginning administration of the compounds or compositions provided herein. For example, the viral load is decreased by about 0.5 log 10, about 1 log 10, about 1.5 log 10, about 2 log 10, or about 2.5 log 10 after administration of the compounds or compositions provided herein, for a certain amount of time as compared to the viral load at the time of beginning administration of the compounds or compositions provided herein. In some embodiments, the viral load is decreased by about 0.5 log 10 after administration of the compounds or compositions provided herein, for about 24 weeks as compared to the viral load at the time of beginning administration of the compounds or compositions provided herein. In some embodiments, the viral load is decreased by about 1 log 10 after administration of the compounds or compositions provided herein, for about 24 weeks as compared to the viral load at the time of beginning administration of the compounds or compositions provided herein. In some embodiments, the viral load is decreased by about 1.5 log 10 after administration of the compounds or compositions provided herein, for about 24 weeks as compared to the viral load at the time of beginning administration of the compounds or compositions provided herein. In some embodiments, the viral load is decreased by about 2 log 10 after administration of the compounds or compositions provided herein, for about 24 weeks as compared to the viral load at the time of beginning administration of the compounds or compositions provided herein. In some embodiments, the viral load is decreased by about 2.5 log 10 after administration of the compounds or compositions provided herein, for about 24 weeks as compared to the viral load at the time of beginning administration of the compounds or compositions provided herein.

In some embodiments of the disclosed methods, the viral load in the patient is about 200 c/mL or less after administration of the compounds or compositions provided herein, for a certain amount of time, such as about 175 c/mL or less, about 150 c/mL or less, about 125 c/mL or less, about 100 c/mL or less, about 75 c/mL or less, or about 50 c/mL or less. In some embodiments, the viral load in the patient is about 200 c/mL or less after administration of the compounds or compositions provided herein, for about 24 weeks. In some embodiments, the viral load in the patient is about 200 c/mL or less after administration of the compounds or compositions provided herein, for about 24 weeks. In some embodiments, the viral load in the patient is about 100 c/mL or less after administration of the compounds or compositions provided herein, for about 24 weeks. In some embodiments, the viral load in the patient is about 50 c/mL or less after administration of the compounds or compositions provided herein, for about 24 weeks.

Also provided in this disclosure is a method of treating an HIV-1 infection in a heavily treatment-experienced patient with multidrug resistant HIV-1 that includes administering a therapeutically effective amount of the compounds, salts, or compositions provided herein, to a patient that had been previously treated with an HIV treatment regimen that includes the administration of at least one antiretroviral medication and had failed the treatment regimen. In some embodiments, the HIV treatment regimen includes administration of at least one antiretroviral medication such as those described herein. In some embodiments of the method, administration of the compounds, salts, or compositions provided herein results in a reduction in HIV viral load in the patient.

Also disclosed is a method of treating an HIV-1 infection in a heavily treatment-experienced patient with multidrug resistant HIV-1 that includes administering a therapeutically effective amount of the compounds, salts, or compositions provided herein to a patient that had been previously treated with an HIV treatment regimen that includes the administration of at least one antiretroviral medication and had failed the treatment regimen, where the multidrug resistant HIV-1 is resistant to at least one antiretroviral medication from each of two different classes of antiretroviral medications. In some embodiments, the different classes of antiretroviral medications are selected from an NRTI, an NNRTI, a PI, and an INSTI. In some embodiments, the patient has a viral load of greater than about 200 c/mL at the time of beginning administration of the compounds or compositions provided herein, and administration of the compound results in a reduction in HIV viral load in the patient.

In some embodiments, disclosed is a method of treating an HIV-1 infection in a heavily treatment-experienced patient with multidrug resistant HIV-1 that includes administering a therapeutically effective amount of the compounds or compositions provided herein, to a patient that had been previously treated with an HIV treatment regimen that includes the administration of at least one antiretroviral medication, and had failed the treatment regimen, where the multidrug resistant HIV-1 is resistant to at least one antiretroviral medication from each of three different classes of antiretroviral medications. In some embodiments, the different classes of antiretroviral medications are selected from an NRTI, an NNRTI, a PI, and an INSTI. In some embodiments, the patient has a viral load of greater than about 200 c/mL at the time of beginning administration of the compounds or compositions provided herein, and administration of the compound results in a reduction in HIV viral load in the patient.

In some embodiments, the methods provided herein comprise preventing a human immunodeficiency virus (HIV) infection in the patient. In some embodiments, the patient may be at risk of contracting an HIV infection. In some embodiments, the patient has been identified as an individual who is at risk of sexual transmission of HIV. In some embodiments, the individual has been identified as a man (e.g., who has sexual intercourse with a man or a woman), transgender man, transgender woman, a woman (e.g., who has sexual intercourse with a man or a woman), as a gender non-binary individual (e.g., who has sexual intercourse with a man or a woman), and/or a sex worker.

As used herein, an “at risk” individual is an individual who is at risk of developing a condition to be treated. An individual “at risk” may or may not have detectable disease or condition, and may or may not have displayed detectable disease prior to the methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

In some embodiments, the individual has been identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual has been identified as having sex with partners of unknown HIV status.

In some embodiments, the individual has been identified as having sex with people who are living with HIV but not on HIV treatment and with an undetectable viral load.

In some embodiments, the individual has been identified as having sex in a geographic area where HIV is common.

In some embodiments, the individual has been identified as having sex in a geographic area of North America (e.g., United States, Canada, Mexico) where HIV is common. In some embodiments, the individual has been identified as having sex in a geographic area of the United States where HIV is common. In some embodiments, the individual has been identified as having sex in a geographic area of the Canada where HIV is common. In some embodiments, the individual has been identified as having sex in a geographic area of the Mexico where HIV is common.

In some embodiments, the individual has been identified as having sex in a geographic area of Africa where HIV is common. In some embodiments, the individual has been identified as having sex in a geographic area of South Africa where HIV is common. In some embodiments, the individual has been identified as having sex in a geographic area of Uganda where HIV is common.

In some embodiments, the individual has been identified as having sex in a geographic area of Europe where HIV is common.

In some embodiments, the individual has been identified as having sex in a geographic area of Asia (e.g., Thailand) where HIV is common. In some embodiments, the individual has been identified as having sex in a geographic area of Thailand where HIV is common.

In some embodiments, the individual has been identified as having sex in a geographic area of South America (e.g., Argentina, Brazil, Peru, and the like) where HIV is common. In some embodiments, the individual has been identified as having sex in a geographic area of Argentina where HIV is common. In some embodiments, the individual has been identified as having sex in a geographic area of Brazil where HIV is common. In some embodiments, the individual has been identified as having sex in a geographic area of Peru where HIV is common.

In some embodiments, the individual has been identified as having sex while under the influence of substances or alcohol.

In some embodiments, the individual has been identified as not using condoms consistently with partners of unknown status.

In some embodiments, the individual has been identified as an individual who injects drugs.

In some embodiments, the individual is a cisgender woman. In some embodiments, the individual is an adolescent cisgender woman. In some embodiments, the adolescent cisgender woman is about 16 to about 25 years of age, for example, about 16, about 17, about 18, about 19, about 20, about 21, about 22, about 23, about 24, about 25, or about 26 years of age.

In some embodiments, the individual is a cisgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender woman identified as having sex with partners of unknown HIV status.

In some embodiments, the individual is a cisgender woman identified as having sex with people who are living with HIV but not on HIV treatment and with an undetectable viral load.

In some embodiments, the individual is a cisgender woman identified as having sex in a geographic area where HIV is common.

In some embodiments, the individual is a cisgender woman identified as having sex in a geographic area of North America (e.g., United States, Canada, Mexico) where HIV is common. In some embodiments, the individual is a cisgender woman identified as having sex in a geographic area of the United States where HIV is common. In some embodiments, the individual is a cisgender woman identified as having sex in a geographic area of the Canada where HIV is common. In some embodiments, the individual is a cisgender woman identified as having sex in a geographic area of the Mexico where HIV is common.

In some embodiments, the individual is a cisgender woman identified as having sex in a geographic area of Africa where HIV is common. In some embodiments, the individual is a cisgender woman identified as having sex in a geographic area of South Africa where HIV is common. In some embodiments, the individual is a cisgender woman identified as having sex in a geographic area of Uganda where HIV is common.

In some embodiments, the individual is a cisgender woman identified as having sex in a geographic area of Europe where HIV is common.

In some embodiments, the individual is a cisgender woman identified as having sex in a geographic area of Asia (e.g., Thailand) where HIV is common. In some embodiments, the individual is a cisgender woman identified as having sex in a geographic area of Thailand where HIV is common.

In some embodiments, the individual is a cisgender woman identified as having sex in a geographic area of South America (e.g., Argentina, Brazil, Peru, and the like) where HIV is common. In some embodiments, the individual is a cisgender woman identified as having sex in a geographic area of Argentina where HIV is common. In some embodiments, the individual is a cisgender woman identified as having sex in a geographic area of Brazil where HIV is common. In some embodiments, the individual is a cisgender woman identified as having sex in a geographic area of Peru where HIV is common.

In some embodiments, the individual is a cisgender woman identified as having sex while under the influence of substances or alcohol.

In some embodiments, the individual is a cisgender woman identified as not using condoms consistently with partners of unknown status.

In some embodiments, the individual is a cisgender woman identified as an individual who injects drugs.

In some embodiments, the individual is a transgender woman. In some embodiments, the individual is an adolescent transgender woman. In some embodiments, the adolescent transgender woman is about 16 to about 25 years of age, for example, about 16, about 17, about 18, about 19, about 20, about 21, about 22, about 23, about 24, about 25, or about 26 years of age.

In some embodiments, the individual is a transgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender woman identified as having sex with partners of unknown HIV status.

In some embodiments, the individual is a transgender woman identified as having sex with people who are living with HIV but not on HIV treatment and with an undetectable viral load.

In some embodiments, the individual is a transgender woman identified as having sex in a geographic area where HIV is common.

In some embodiments, the individual is a transgender woman identified as having sex in a geographic area of North America (e.g., United States, Canada, Mexico) where HIV is common. In some embodiments, the individual is a transgender woman identified as having sex in a geographic area of the United States where HIV is common. In some embodiments, the individual is a transgender woman identified as having sex in a geographic area of the Canada where HIV is common. In some embodiments, the individual is a transgender woman identified as having sex in a geographic area of the Mexico where HIV is common.

In some embodiments, the individual is a transgender woman identified as having sex in a geographic area of Africa where HIV is common. In some embodiments, the individual is a transgender woman identified as having sex in a geographic area of South Africa where HIV is common. In some embodiments, the individual is a transgender woman identified as having sex in a geographic area of Uganda where HIV is common.

In some embodiments, the individual is a transgender woman identified as having sex in a geographic area of Europe where HIV is common.

In some embodiments, the individual is a transgender woman identified as having sex in a geographic area of Asia (e.g., Thailand) where HIV is common. In some embodiments, the individual is a transgender woman identified as having sex in a geographic area of Thailand where HIV is common.

In some embodiments, the individual is a transgender woman identified as having sex in a geographic area of South America (e.g., Argentina, Brazil, Peru, and the like) where HIV is common. In some embodiments, the individual is a transgender woman identified as having sex in a geographic area of Argentina where HIV is common. In some embodiments, the individual is a transgender woman identified as having sex in a geographic area of Brazil where HIV is common. In some embodiments, the individual is a transgender woman identified as having sex in a geographic area of Peru where HIV is common.

In some embodiments, the individual is a transgender woman identified as having sex while under the influence of substances or alcohol.

In some embodiments, the individual is a transgender woman identified as not using condoms consistently with partners of unknown status.

In some embodiments, the individual is a transgender woman identified as an individual who injects drugs.

In some embodiments, the individual is a cisgender man. In some embodiments, the individual is an adolescent cisgender man. In some embodiments, the adolescent cisgender man is about 16 to about 25 years of age, for example, about 16, about 17, about 18, about 19, about 20, about 21, about 22, about 23, about 24, about 25, or about 26 years of age.

In some embodiments, the individual is a cisgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender man identified as having sex with partners of unknown HIV status.

In some embodiments, the individual is a cisgender man identified as having sex with people who are living with HIV but not on HIV treatment and with an undetectable viral load. In some embodiments, the individual is a cisgender man identified as having sex in a geographic area where HIV is common.

In some embodiments, the individual is a cisgender man identified as having sex in a geographic area of North America (e.g., United States, Canada, Mexico) where HIV is common. In some embodiments, the individual is a cisgender man identified as having sex in a geographic area of the United States where HIV is common. In some embodiments, the individual is a cisgender man identified as having sex in a geographic area of the Canada where HIV is common. In some embodiments, the individual is a cisgender man identified as having sex in a geographic area of the Mexico where HIV is common.

In some embodiments, the individual is a cisgender man identified as having sex in a geographic area of Africa where HIV is common. In some embodiments, the individual is a cisgender man identified as having sex in a geographic area of South Africa where HIV is common. In some embodiments, the individual is a cisgender man identified as having sex in a geographic area of Uganda where HIV is common.

In some embodiments, the individual is a cisgender man identified as having sex in a geographic area of Europe where HIV is common.

In some embodiments, the individual is a cisgender man identified as having sex in a geographic area of Asia (e.g., Thailand) where HIV is common. In some embodiments, the individual is a cisgender man identified as having sex in a geographic area of Thailand where HIV is common.

In some embodiments, the individual is a cisgender man identified as having sex in a geographic area of South America (e.g., Argentina, Brazil, Peru, and the like) where HIV is common. In some embodiments, the individual is a cisgender man identified as having sex in a geographic area of Argentina where HIV is common. In some embodiments, the individual is a cisgender man identified as having sex in a geographic area of Brazil where HIV is common. In some embodiments, the individual is a cisgender man identified as having sex in a geographic area of Peru where HIV is common.

In some embodiments, the individual is a cisgender man identified as having sex while under the influence of substances or alcohol.

In some embodiments, the individual is a cisgender man identified as not using condoms consistently with partners of unknown status.

In some embodiments, the individual is a cisgender man identified as an individual who injects drugs.

In some embodiments, the individual is a transgender man. In some embodiments, the individual is an adolescent transgender man. In some embodiments, the adolescent transgender man is about 16 to about 25 years of age, for example, about 16, about 17, about 18, about 19, about 20, about 21, about 22, about 23, about 24, about 25, or about 26 years of age.

In some embodiments, the individual is a transgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender man identified as having sex with partners of unknown HIV status.

In some embodiments, the individual is a transgender man identified as having sex with people who are living with HIV but not on HIV treatment and with an undetectable viral load.

In some embodiments, the individual is a transgender man identified as having sex in a geographic area where HIV is common.

In some embodiments, the individual is a transgender man identified as having sex in a geographic area of North America (e.g., United States, Canada, Mexico) where HIV is common. In some embodiments, the individual is a transgender man identified as having sex in a geographic area of the United States where HIV is common. In some embodiments, the individual is a transgender man identified as having sex in a geographic area of the Canada where HIV is common. In some embodiments, the individual is a transgender man identified as having sex in a geographic area of the Mexico where HIV is common.

In some embodiments, the individual is a transgender man identified as having sex in a geographic area of Africa where HIV is common. In some embodiments, the individual is a transgender man identified as having sex in a geographic area of South Africa where HIV is common. In some embodiments, the individual is a transgender man identified as having sex in a geographic area of Uganda where HIV is common.

In some embodiments, the individual is a transgender man identified as having sex in a geographic area of Europe where HIV is common.

In some embodiments, the individual is a transgender man identified as having sex in a geographic area of Asia (e.g., Thailand) where HIV is common. In some embodiments, the individual is a transgender man identified as having sex in a geographic area of Thailand where HIV is common.

In some embodiments, the individual is a transgender man identified as having sex in a geographic area of South America (e.g., Argentina, Brazil, Peru, and the like) where HIV is common. In some embodiments, the individual is a transgender man identified as having sex in a geographic area of Argentina where HIV is common. In some embodiments, the individual is a transgender man identified as having sex in a geographic area of Brazil where HIV is common. In some embodiments, the individual is a transgender man identified as having sex in a geographic area of Peru where HIV is common.

In some embodiments, the individual is a transgender man identified as having sex while under the influence of substances or alcohol.

In some embodiments, the individual is a transgender man identified as not using condoms consistently with partners of unknown status.

In some embodiments, the individual is a transgender man identified as an individual who injects drugs.

In some embodiments, the individual is a gender non-binary individual. In some embodiments, the individual is an adolescent gender non-binary individual. In some embodiments, the adolescent gender non-binary individual is about 16 to about 25 years of age, for example, about 16, about 17, about 18, about 19, about 20, about 21, about 22, about 23, about 24, about 25, or about 26 years of age.

In some embodiments, the individual is a gender non-binary individual identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a gender non-binary individual identified as having sex with partners of unknown HIV status.

In some embodiments, the individual is a gender non-binary individual identified as having sex with people who are living with HIV but not on HIV treatment and with an undetectable viral load.

In some embodiments, the individual is a gender non-binary individual identified as having sex in a geographic area where HIV is common.

In some embodiments, the individual is a gender non-binary individual identified as having sex in a geographic area of North America (e.g., United States, Canada, Mexico) where HIV is common. In some embodiments, the individual is a gender non-binary individual identified as having sex in a geographic area of the United States where HIV is common. In some embodiments, the individual is a gender non-binary individual identified as having sex in a geographic area of the Canada where HIV is common. In some embodiments, the individual is a gender non-binary individual identified as having sex in a geographic area of the Mexico where HIV is common.

In some embodiments, the individual is a gender non-binary individual identified as having sex in a geographic area of Africa where HIV is common. In some embodiments, the individual is a gender non-binary individual identified as having sex in a geographic area of South Africa where HIV is common. In some embodiments, the individual is a gender non-binary individual identified as having sex in a geographic area of Uganda where HIV is common.

In some embodiments, the individual is a gender non-binary individual identified as having sex in a geographic area of Europe where HIV is common.

In some embodiments, the individual is a gender non-binary individual identified as having sex in a geographic area of Asia (e.g., Thailand) where HIV is common. In some embodiments, the individual is a gender non-binary individual identified as having sex in a geographic area of Thailand where HIV is common.

In some embodiments, the individual is a gender non-binary individual identified as having sex in a geographic area of South America (e.g., Argentina, Brazil, Peru, and the like) where HIV is common. In some embodiments, the individual is a gender non-binary individual identified as having sex in a geographic area of Argentina where HIV is common. In some embodiments, the individual is a gender non-binary individual identified as having sex in a geographic area of Brazil where HIV is common. In some embodiments, the individual is a gender non-binary individual identified as having sex in a geographic area of Peru where HIV is common.

In some embodiments, the individual is a gender non-binary individual identified as having sex while under the influence of substances or alcohol.

In some embodiments, the individual is a gender non-binary individual identified as not using condoms consistently with partners of unknown status.

In some embodiments, the individual is a gender non-binary individual identified as an individual who injects drugs.

In some embodiments, the individual has been identified as:
having anal sex with at least two different sexual partners and no consistent condom use over the last 6 months; and/or
having history of sexually transmitted diseases (STDs) during the last 12 months (e.g., syphilis, gonorrhea, chlamydiae, HBV or HCV infection); and/or
using psycho-active drugs during sexual intercourses (e.g., cocaine, gammahydroxybutyric acid (GHB), methylenedioxymethamphetamine (MDMA), mephedrone); and/or
having sexual intercourse with one or more partners originating from a region with high prevalence of HIV infection (>1%) (e.g., South America, Sub-Saharan Africa, South-East Asia, Eastern Europe, French Guyana) and no consistent condom use; and/or
a sex worker; and/or
having a sexual partner who is an intravenous drug user sharing injection material; and/or
having an HIV-infected sexual partner with a detectable plasma viral load (e.g., >50 copies (cp)/milliliter (mL)); and/or
a cisgender man;
a transgender man;
a cisgender woman;
a transgender woman;
a gender non-binary individual; and/or
a person who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the methods provided herein comprise administering to the patient a prophylactically effective amount of the compound of Formula Ia or Ib, or a pharmaceutically acceptable salt thereof, and a compound of Formula II, or a pharmaceutically acceptable salt thereof, for pre-exposure prophylaxis (PrEP) to prevent sexually acquired HIV-1 in adults and adolescents. In some embodiments, the methods provided herein comprise administering to the patient a prophylactically effective amount of the compound of Formula Ia or Ib, or a pharmaceutically acceptable salt thereof, and a compound of Formula II, or a pharmaceutically acceptable salt thereof, for pre-exposure prophylaxis (PrEP) to prevent sexually acquired HIV-1 in adults and adolescents weighing at least 35 kg. In some embodiments, the patient has obtained a negative HIV-1 test prior to administration of the compound of Formula Ia or Ib, or a pharmaceutically acceptable salt thereof, and a compound of Formula II, or a pharmaceutically acceptable salt thereof.

In some embodiments, the patient is HIV-negative. In some embodiments, the HIV is HIV-1. In some embodiments, the HIV is HIV-2. In some embodiments, the HIV is HIV-1 and HIV-2.

As used herein, “HIV” or “Human Immunodeficiency Virus” refers to HIV-1 and/or to HIV-2.

The term “patient” is meant to refer to a human who is in need of preventative treatment for a viral infection, such as HIV infection.

As used herein, the terms “prevention” or “preventing” refer to the administration of a compound, or a pharmaceutically acceptable salt or free acid form thereof, or composition comprising the compound, pharmaceutically acceptable salt, or free acid form thereof according to the present disclosure pre- or post-exposure of the patient to the virus but before the appearance of symptoms of the disease, and/or prior to the detection of the virus in the blood. The terms also refer to prevention of the appearance of symptoms of the disease and/or to prevent the virus from reaching detectable levels in the blood. The terms include both pre-exposure prophylaxis (PrEP), as well as post-exposure prophylaxis (PEP) and event driven or “on demand” prophylaxis. The terms also refer to prevention of perinatal transmission of HIV from mother to baby by administration of a compound, pharmaceutically acceptable salt thereof, or composition comprising the compound or the pharmaceutically acceptable salt thereof according to the present disclosure to the mother before giving birth and to the child within the first days of life. The term also refers to prevention of transmission of HIV through blood transfusion.

As used herein, the term “period of exposure” refers to a period of time, ranging from a single event or to multiple events over an extended period of time, in which a patient is exposed to HIV. For example, a patient who engages in one sexual intercourse event with a partner who is HIV-positive has a period of exposure that is limited to the time and duration of that one sexual intercourse event with that partner. As another example, a patient who has sexual intercourse with a partner who is HIV-positive on multiple occasions over an extended period of time (e.g., days, weeks, months, or years) has a period of exposure that ranges from the first instance to the last instance of sexual intercourse with that partner.

In some embodiments, the methods disclosed herein may comprise event driven administration of a composition provided herein (e.g., a composition comprising a compound of Formula Ia or Ib, or a pharmaceutically acceptable salt or free acid form thereof), to the patient. As used herein, the terms “event driven” or “event driven administration” refer to administration of a composition provided herein, (1) prior to an event (e.g., 2 hours, 1 day, 2 days, 5 days, 7 days, 10 days, 14 days, 28 days (i.e., one month), or more days prior to the event) that would expose the patient to HIV (or that would otherwise increase the patient's risk of acquiring HIV); and/or (2) during an event (or more than one recurring event) that would expose the patient to HIV (or that would otherwise increase the patient's risk of acquiring HIV); and/or (3) after an event (or after the final event in a series of recurring events) that would expose the patient to HIV (or that would otherwise increase the patient's risk of acquiring HIV). In some embodiments, the event driven administration is performed pre-exposure of the patient to the HIV. In some embodiments, the event driven administration is performed during exposure of the patient to the HIV. In some embodiments, the event driven administration is performed post-exposure of the patient to the HIV.

In some embodiments, the event driven administration is performed pre-exposure of the patient to the HIV and during exposure of the patient to the HIV.

In some embodiments, the event driven administration is performed pre-exposure of the patient to the HIV and post-exposure of the patient to the HIV.

In some embodiments, the event driven administration is performed during exposure of the patient to the HIV and post-exposure of the patient to the HIV.

In certain embodiments, the methods disclosed herein involve administration prior to and/or after an event that would expose the patient to HIV or that would otherwise increase the patient's risk of acquiring HIV, e.g., as pre-exposure prophylaxis (PrEP) and/or as post-exposure prophylaxis (PEP). Examples of events that could increase a patient's risk of acquiring HIV include, without limitation, no condom use during anal intercourse with an HIV positive partner or a partner of unknown HIV status; anal intercourse with more than 3 sexual partners; exchange of money, gifts, shelter or drugs for anal sex; sex with male partner and diagnosis of sexually transmitted infection; and no consistent use of condoms with a sexual partner known to be HIV positive. In some embodiments, the methods disclosed herein comprise pre-exposure prophylaxis (PrEP). In some embodiments, methods disclosed herein comprise post-exposure prophylaxis (PEP). In some embodiments, the methods disclosed herein comprise pre-exposure prophylaxis (PrEP) and post-exposure prophylaxis (PEP).

In some embodiments, a composition provided herein is administered before exposure of the patient to the HIV.

In some embodiments, a composition provided herein is administered during the period of exposure of the patient to the HIV.

In some embodiments, a composition provided herein is administered after final exposure of the patient to the HIV.

In some embodiments, a composition provided herein is administered before and during exposure of the patient to the HIV.

In some embodiments, a composition provided herein is administered before and after exposure of the patient to the HIV.

In some embodiments, a composition provided herein is administered during and after exposure of the patient to the HIV.

In some embodiments, a composition provided herein is administered before, during, and after exposure of the patient to the HIV.

In some embodiments, the dose of the composition administered during each period (i.e., before, during, and after exposure) may be different, i.e, independently selected from any of the doses disclosed herein.

In certain embodiments, e.g., when administered as PrEP, a composition provided herein is administered 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity, prior to sexual intercourse or other exposure to the HIV). In some embodiments, a composition provided herein is administered within 14 days, 13 days, 12 days, 11 days, 10 days, 9 days, 8 days, 7 days, 6 days, 5 days, 4 days, 3 days, 2 days, or 1 day prior to an event that would increase the patient's risk of acquiring HIV (e.g., prior to sexual intercourse or other exposure to the HIV). In some embodiments, a composition provided herein is administered within 72 hours, 60 hours, 48 hours, 24 hours, 12 hours, 9 hours, 6 hours, 4 hours, 3 hours, 2 hours, or 1 hour prior to an event that would increase the patient's risk of acquiring HIV (e.g., prior to sexual intercourse or other exposure to the HIV). In certain embodiments, when a composition provided herein is administered prior to an event that would increase the patient's risk of acquiring HIV, it is administered daily prior to the event (e.g., sexual activity). In certain embodiments, when a composition provided herein is administered prior to an event that would increase the patient's risk of acquiring HIV, it is administered one to three times prior to the event. In certain embodiments, when a composition provided herein is administered prior to an event that would increase the patient's risk of acquiring HIV, it is administered one time (i.e., once) prior to the event.

In some embodiments, a composition provided herein is administered once from about 31 days to about one day before exposure of the patient to the HIV. In some embodiments, a composition provided herein is administered once from about 30 days to about one day before exposure of the patient to the HIV. In some embodiments, a composition provided herein is administered from about 14 days to about one day before exposure of the patient to the HIV. In some embodiments, a composition provided herein is administered once from about 14 days to about one day before exposure of the patient to the HIV.

In some embodiments, a composition provided herein is administered from about 10 days to about 5 days before exposure of the patient to the HIV. In some embodiments, a composition provided herein is administered once from about 10 days to about 5 days before exposure of the patient to the HIV.

In some embodiments, a composition provided herein is administered from about 8 days to about 6 days before exposure of the patient to the HIV. In some embodiments, a composition provided herein is administered once from about 8 days to about 6 days before exposure of the patient to the HIV.

In some embodiments, a composition provided herein is administered about 7 days before exposure of the patient to the HIV. In some embodiments, a composition provided herein is administered once about 7 days before exposure of the patient to the HIV.

In some embodiments, a composition provided herein is administered from about 72 hours to about 1 hour before exposure of the patient to the HIV. In some embodiments, a composition provided herein is administered once from about 72 hours to about 1 hour before exposure of the patient to the HIV.

In some embodiments of the methods provided herein, the pre-exposure prophylaxis (PrEP) comprises continuous PrEP.

In certain embodiments where a composition provided herein is administered before exposure of the patient to the HIV, the methods disclosed herein further comprise administering one or more additional doses of the compound of Formula Ia or Ib, or a pharmaceutically acceptable salt or free acid form thereof, during, and/or after exposure of the patient to the HIV.

In some embodiments, e.g., when administered as part of a PrEP regimen or as part of a PEP regimen, a composition provided herein is administered during the period of exposure of the patient to the HIV. In certain embodiments wherein a composition provided herein is administered before HIV exposure, the composition is administered about every 7 days, about every 14 days, about every 21 days, about every 28 days, about every 35 days, about every 42 days, or about every 6 months, or about every 12 months (e.g., as a single dose) during the time of HIV exposure (e.g., during the time period of sexual activity with a sexual partner known to be HIV positive). In some embodiments, a composition provided herein is administered once about every 7 days, about every 14 days, about every 21 days, about every 28 days, about every 35 days, about every 42 days, about every 6 months, or about every 12 months during the period of exposure of the patient to the HIV.

In some embodiments, a composition provided herein administered prior to exposure to the HIV is at a different dose than a composition (e.g., a composition comprising a compound of Formula Ia or Ib, or a pharmaceutically acceptable salt or free acid form thereof) administered during and/or after exposure to the HIV. For example, in some embodiments, the dose of the compound of Formula Ia or Ib, or a pharmaceutically acceptable salt or free acid form thereof, is increased, e.g., as a double dose, as a triple dose, and the like as compared to an earlier administered dose (e.g., a dose prior to exposure to the HIV). In some embodiments, the increased dose of the compound of Formula Ia or Ib, or a pharmaceutically acceptable salt or free acid form thereof, is a double dose. In some embodiments, the dose of the compound of Formula Ia or Ib, or a pharmaceutically acceptable salt or free acid form thereof, is decreased, e.g., a half dose as compared to an earlier administered dose (e.g., a dose prior to exposure to the HIV).

In some embodiments, a composition provided herein (e.g., a composition comprising a compound of Formula Ia or Ib, or a pharmaceutically acceptable salt or free acid form thereof) is administered as a single dose from about 1 hour to about 10 days before exposure of the patient to the HIV.

Additional examples of PrEP and/or PEP can be found, for example, at the clinical trial summary titled “On Demand Antiretroviral Pre-exposure Prophylaxis for HIV Infection in Men Who Have Sex With Men” (Clinical Trial #NCT01473472); the clinical trial summary titled “Prevention of HIV in Île-de-France” (Clinical Trials #NCT03113123), and at Molina et al, N. Engl. J. Med. 2015, 353:2237-2246, the disclosure of each of which is incorporated herein by reference in its entirety.

In some embodiments, e.g., when administered as part of a PrEP regimen or as part of a PEP regimen, a composition provided herein is administered 1 hour to 10 days, 1 hour to 7 days, 1 hour to 5 days, 1 to 72 hours, 1 to 48 hours, 1 to 36 hours, 1 to 24 hours, or 1 to 12 hours following an event that would increase the patient's risk of acquiring HIV (e.g., following sexual intercourse or other exposure to the HIV).

In certain embodiments, e.g., when administered as PEP, a composition provided herein is administered for 7 days, 14 days, 21 days, 28 days, 30 days, or 45 days following an event that would increase the patient's risk of acquiring HIV (e.g., following sexual intercourse or other exposure to the HIV). In certain embodiments, e.g., when administered as PEP, a composition provided herein is administered for 30 days following an event that would increase the patient's risk of acquiring HIV (e.g., following sexual intercourse or other exposure to the HIV). In certain embodiments, a composition provided herein is administered less than 1 hour, 2 hours, 3 hours, 4 hours, 5 hours, 6 hours, 7 hours, 8 hours, 9 hours, 12 hours, 18 hours, 24 hours, 36 hours, or 48 hours following an event that would increase the patient's risk of acquiring HIV (e.g., following sexual intercourse or other exposure to the HIV virus). In certain embodiments, a composition provided herein is administered for 1 day, 2 days, 3 days, 4 days, or 5 days following an event that would increase the patient's risk of acquiring HIV (e.g., following sexual intercourse or other exposure to the HIV). In certain embodiments, when a composition provided herein is administered following an event that would increase the patient's risk of acquiring HIV, it is administered daily following the event. In certain embodiments, when a composition provided herein is administered following an event that would increase the patient's risk of acquiring HIV, it is administered one to three times following the event. In certain embodiments, when a composition provided herein is administered following an event that would increase the patient's risk of acquiring HIV, it is administered once following the event. In certain embodiments, e.g., when administered as PEP, a composition provided herein is administered once about every week, once about every month, once about every 2 months, once about every 3 months, once about every 4 months, once about every 5 months, once about every 6 months, or once about every 12 months following an event that would increase the patient's risk of acquiring HIV (e.g., following sexual intercourse or other exposure to the HIV). In certain embodiments, e.g., when administered as PEP, a composition provided herein is administered once about every week following an event that would increase the patient's risk of acquiring HIV (e.g., following sexual intercourse or other exposure to the HIV). In certain embodiments, e.g., when administered as PEP, a composition provided herein is administered once about every month following an event that would increase the patient's risk of acquiring HIV (e.g., following sexual intercourse or other exposure to the HIV). In certain embodiments, e.g., when administered as PEP, a composition provided herein is administered once about every 2 months following an event that would increase the patient's risk of acquiring HIV (e.g., following sexual intercourse or other exposure to the HIV). In certain embodiments, e.g., when administered as PEP, a composition provided herein is administered once about every 3 months following an event that would increase the patient's risk of acquiring HIV (e.g., following sexual intercourse or other exposure to the HIV). In certain embodiments, e.g., when administered as PEP, a composition provided herein is administered once about every 4 months following an event that would increase the patient's risk of acquiring HIV (e.g., following sexual intercourse or other exposure to the HIV). In certain embodiments, e.g., when administered as PEP, a composition provided herein is administered once about every 5 months following an event that would increase the patient's risk of acquiring HIV (e.g., following sexual intercourse or other exposure to the HIV). In certain embodiments, e.g., when administered as PEP, a composition provided herein is administered once about every 6 months following an event that would increase the patient's risk of acquiring HIV (e.g., following sexual intercourse or other exposure to the HIV). In certain embodiments, e.g., when administered as PEP, a composition provided herein is administered once about every 12 months following an event that would increase the patient's risk of acquiring HIV (e.g., following sexual intercourse or other exposure to the HIV).

In certain embodiments, e.g., when administered as PEP, a composition provided herein is administered for one month, two months, three months, four months, five months, six months, or twelve months following an event that would increase the patient's risk of acquiring HIV (e.g., following sexual intercourse or other exposure to the HIV).

In certain embodiments, when a composition provided herein is administered following an event that would increase the patient's risk of acquiring HIV, it is administered one to fifty times following the event. In certain embodiments, when a composition provided herein is administered following an event that would increase the patient's risk of acquiring HIV, it is administered one to forty times following the event. In certain embodiments, when a composition provided herein is administered following an event that would increase the patient's risk of acquiring HIV, it is administered one to thirty times following the event. In certain embodiments, when a composition provided herein is administered following an event that would increase the patient's risk of acquiring HIV, it is administered one to twenty times following the event. In certain embodiments, when a composition provided herein is administered following an event that would increase the patient's risk of acquiring HIV, it is administered one to fifteen times following the event. In certain embodiments, when a composition provided herein is administered following an event that would increase the patient's risk of acquiring HIV, it is administered one to ten times following the event. In certain embodiments, when a composition provided herein is administered following an event that would increase the patient's risk of acquiring HIV, it is administered one to five times following the event.

In some embodiments, a composition provided herein is administered during exposure of the patient to the HIV (e.g., during a period of sexual activity with a sexual partner known to be HIV positive).

In some embodiments, a composition provided herein is administered after exposure (e.g., after final exposure) of the patient to the HIV (e.g., after a period of sexual activity with a sexual partner known to be HIV positive). In some embodiments, a composition provided herein is administered from about 1 hour to about 14 days after exposure (e.g., after final exposure) of the patient to the HIV. In some embodiments, a composition provided herein is administered once from about 1 hour to about 14 days after exposure (e.g., after final exposure) of the patient to the HIV. In some embodiments, a composition provided herein is administered from about 1 hour to about 7 days after exposure (e.g., after final exposure) of the patient to the HIV. In some embodiments, a composition provided herein is administered once from about 1 hour to about 7 days after exposure (e.g., after final exposure) of the patient to the HIV. In some embodiments, a composition provided herein is administered from about 1 hour to about 72 hours after exposure (e.g., after final exposure) of the patient to the HIV. In some embodiments, a composition provided herein is administered once from about 1 hour to about 72 hours after exposure (e.g., after final exposure) of the patient to the HIV. In some embodiments, a composition provided herein is administered from about 1 hour to about 24 hours after exposure (e.g., after final exposure) of the patient to the HIV. In some embodiments, a composition provided herein is administered once from about 1 hour to about 24 hours after exposure (e.g., after final exposure) of the patient to the HIV. In some embodiments, a composition provided herein is administered from about 24 hours to about 72 hours after exposure (e.g., after final exposure) of the patient to the HIV. In some embodiments, a composition provided herein is administered once from about 24 hours to about 72 hours after exposure (e.g., after final exposure) of the patient to the HIV.

In some embodiments, e.g., when administered as PrEP, a composition provided herein is administered prior to an event that would increase the patient's risk of acquiring HIV (e.g., prior to sexual activity), and following the event. For example, in certain embodiments, when administered as PrEP, a composition provided herein is administered 1 to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) and 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hourto 36 hours, 1 hour to 24 hours, or 1 hour to 12 hours following the event. For example, in some embodiments, one or more (e.g., one, two, or three) dosages of a composition provided herein are administered one to ten days (e.g., seven days) prior to an event that would increase the patient's risk of acquiring HIV (e.g., prior to sexual intercourse) and once during a period of one to ten days following the event. In some embodiments, a composition provided herein is administered once per week, twice per week, three times per week, four times per week, or five times per week and one or more times (e.g., one, two, or three times) beginning 1 to 48 hours following an event that would increase the patient's risk of acquiring HIV (e.g., following sexual intercourse).

Also provided herein is a method of reducing the risk of acquiring HIV in a patient, comprising administering to the patient a composition provided herein (e.g., a composition comprising a compound of Formula Ia or Ib, or a pharmaceutically acceptable salt or free acid form thereof).

In some embodiments, methods for reducing the risk of acquiring HIV (e.g., HIV-1 and/or HIV-2) comprise administration of a composition provided herein to a patient in combination with safer sexual intercourse practices. In certain embodiments, methods for reducing the risk of acquiring HIV (e.g., HIV-1 and/or HIV-2) comprise administration of a composition provided herein to a patient at risk of acquiring HIV. Examples of patients at high risk for acquiring HIV include, without limitation, a patient who is at risk of sexual transmission of HIV.

In some embodiments, the reduction in risk of acquiring HIV is at least about 40%, 50%, 60%, 70%, 80%, 90%, or 95% (compared to a patient having not been administered the compound of Formula Ia or Ib, or a pharmaceutically acceptable salt thereof according to any of the methods provided herein). In some embodiments, the reduction in risk of acquiring HIV is about 80%, 85%, or 90%. In some embodiments, the reduction in risk of acquiring HIV is at least about 75%. In some embodiments, the reduction in risk of acquiring HIV is at least about 80%. In some embodiments, the reduction in risk of acquiring HIV is at least about 85%. In some embodiments, the reduction in risk of acquiring HIV is at least about 90%.

In some embodiments, a composition provided herein (e.g., a tablet comprising a compound of Formula Ia or Ib, or a pharmaceutically acceptable salt or free acid form thereof) is suitable for oral administration. In some embodiments, a composition provided herein (e.g., a tablet comprising a compound of Formula Ia or Ib, or a pharmaceutically acceptable salt or free acid form thereof) is administered orally.

In some embodiments, a composition provided herein (e.g., a tablet comprising a compound of Formula Ia or Ib, or a pharmaceutically acceptable salt or free acid form thereof) is administered once every five to ten days, for example, once every five days, six days, seven days, eight days, nine days, or ten days. In some embodiments, a composition provided herein (e.g., a tablet comprising a compound of Formula Ia or Ib, or a pharmaceutically acceptable salt or free acid form thereof) is administered once every six to eight days. In some embodiments, a composition provided herein (e.g., a tablet comprising a compound of Formula Ia or Ib, or a pharmaceutically acceptable salt or free acid form thereof) is administered once every seven days (i.e., once-weekly).

In some embodiments, a composition provided herein (e.g., a tablet comprising a compound of Formula Ia or Ib, or a pharmaceutically acceptable salt or free acid form thereof) is orally administered once every five to ten days, for example, once every five days, six days, seven days, eight days, nine days, or ten days. In some embodiments, a composition provided herein (e.g., a tablet comprising a compound of Formula Ia or Ib, or a pharmaceutically acceptable salt or free acid form thereof) is orally administered once every six to eight days. In some embodiments, a composition provided herein (e.g., a tablet comprising a compound of Formula Ia or Ib, or a pharmaceutically acceptable salt or free acid form thereof) is orally administered once every seven days (i.e., once-weekly).

In some embodiments, a composition provided herein (e.g., a tablet comprising a compound of Formula Ia or Ib, or a pharmaceutically acceptable salt or free acid form thereof) is orally administered once every month.

The compounds provided herein (e.g., a compound of Formula Ia or Ib and a compound of Formula II), or a salt thereof, can be present in one or more compositions (such as one or more pharmaceutical compositions or formulations) where each composition includes at least one compound other than the active agent (i.e., the compound of Formula Ia or Ib or compound of Formula II), or a salt thereof.

Compositions provided herein can include mixtures containing the active agent, or salt thereof, and one or more solvents, substrates, carriers, etc. In some embodiments, the composition comprises a compound provided herein, or salt thereof, in an amount greater than about 25% by weight, for example, greater than about 25% by weight, greater than about 50% by weight, greater than about 75% by weight, greater than about 80% by weight, greater than about 90% by weight, or greater than about 95% by weight.

The present disclosure further includes pharmaceutical compositions comprising a compound provided herein (e.g., a compound of Formula Ia or Ib, or a compound of Formula II), or a pharmaceutically acceptable salt thereof, and at least one pharmaceutically acceptable carrier. As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Administration of the compounds provided herein, or a pharmaceutically acceptable salt thereof, can be carried out via any of the accepted modes of administration of agents for serving similar utilities. The pharmaceutical compositions of the disclosure can be prepared by combining the active agent, or a pharmaceutically acceptable salt thereof, with an appropriate pharmaceutically acceptable carrier and, in specific embodiments, are formulated into preparations in solid, semi solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral, sublingual, buccal, rectal, vaginal, and intranasal. In some embodiments, pharmaceutical compositions of the disclosure are tablets.

Pharmaceutical compositions of the disclosure are formulated so as to allow the active ingredients contained therein to be bioavailable upon administration of the composition to a patient. Compositions that will be administered to a patient take the form of one or more dosage units, where for example, a tablet may be a single dosage unit. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount or prophylactically effective amount of the active agent, or a pharmaceutically acceptable salt thereof, for treating or preventing an HIV infection, as described herein.

The pharmaceutical compositions disclosed herein can be also prepared by other methodologies known in the pharmaceutical art.

In some embodiments, the methods provided herein comprise administration of:
(a) a pharmaceutical composition comprising a compound of Formula Ia, or a pharmaceutically acceptable salt thereof, and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ia, and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ia, and one or more pharmaceutically acceptable excipients; and
(b) a pharmaceutical composition comprising a compound of Formula II, and one or more pharmaceutically acceptable excipients

In some embodiments, the methods provided herein comprise administration of:
(a) a pharmaceutical composition comprising a compound of Formula Ib, or a pharmaceutically acceptable salt thereof, and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ib, and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ib, and one or more pharmaceutically acceptable excipients; and
(b) a pharmaceutical composition comprising a compound of Formula II, and one or more pharmaceutically acceptable excipients.

In some embodiments, the methods provided herein comprise administration of:
(a) a pharmaceutical composition comprising a crystalline form of the compound of Formula Ia (e.g., crystalline Form I), and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ia, crystalline Form I, and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ia, crystalline Form I, and one or more pharmaceutically acceptable excipients; and
(b) a pharmaceutical composition comprising a compound of Formula II, and one or more pharmaceutically acceptable excipients.

In some embodiments, the methods provided herein comprise administration of:
(a) a pharmaceutical composition comprising a crystalline form of the compound of Formula Ib (e.g., crystalline Form I), and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ib, crystalline Form I, and one or more pharmaceutically acceptable excipients; and
(b) a pharmaceutical composition comprising a compound of Formula II, or a pharmaceutically acceptable salt thereof, and one or more pharmaceutically acceptable excipients.

In some embodiments, the methods provided herein comprise administration of:
(a) a pharmaceutical composition comprising a compound of Formula Ib, crystalline Form I, and one or more pharmaceutically acceptable excipients; and
(b) a pharmaceutical composition comprising a compound of Formula II, and one or more pharmaceutically acceptable excipients.

The compounds provided herein, or pharmaceutically acceptable salts thereof, can be administered by any useful route and means, such as by oral or parenteral (e.g., intravenous) administration. Therapeutically effective amounts of the compound, or a pharmaceutically acceptable salt thereof, may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein, or pharmaceutically acceptable salts thereof, includes from about 0.3 mg to about 30 mg per day, or from about 30 mg to about 300 mg per day, or from about 0.3 μg to about 30 mg per day, or from about 30 μg to about 300 μg per day.

Therapeutically or prophylactically effective dosages of the compounds provided herein (e.g., a compound of Formula Ia or Ib) may include, for example, from about 0.1 mg per dose to about 2400 mg per dose, such as from about 0.1 mg per dose to about 2000 mg per dose, from about 1 mg per dose to about 2400 mg per dose, from about 5 mg per dose to about 2000 mg per dose, from about 10 mg per dose to about 2400 mg per dose, from about 50 mg per dose to about 2400 mg per dose, from about 100 mg per dose to about 2400 mg per dose, from about 200 mg per dose to about 2400 mg per dose, from about 300 mg per dose to about 2400 mg per dose, from about 400 mg per dose to about 2400 mg per dose, from about 500 mg per dose to about 2400 mg per dose, from about 600 mg per dose to about 2400 mg per dose, from about 700 mg per dose to about 2400 mg per dose, from about 800 mg per dose to about 2400 mg per dose, from about 900 mg per dose to about 2400 mg per dose, from about 1000 mg per dose to about 2400 mg per dose, from about 1100 mg per dose to about 2400 mg per dose, from about 1200 mg per dose to about 2400 mg per dose, from about 1300 mg per dose to about 2400 mg per dose, from about 1400 mg per dose to about 2400 mg per dose, from about 1500 mg per dose to about 2400 mg per dose, from about 1600 mg per dose to about 2400 mg per dose, from about 1700 mg per dose to about 2400 mg per dose, from about 1800 mg per dose to about 2400 mg per dose, from about 1900 mg per dose to about 2400 mg per dose, from about 2000 mg per dose to about 2400 mg per dose, from about 2100 mg per dose to about 2400 mg per dose, from about 2200 mg per dose to about 2400 mg per dose, or from about 2300 mg per dose to about 2400 mg per dose.

In some embodiments, a therapeutically or prophylactically effective amount of a compound provided herein (e.g., a compound of Formula Ia or Ib), or a pharmaceutically acceptable salt thereof, is about 50 mg, about 75 mg, about 100 mg, about 125 mg, about 150 mg, about 175 mg, about 200 mg, about 225 mg, about 275 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, about 2000 mg, about 2100 mg, about 2200 mg, about 2300 mg, about 2400 mg.

In some embodiments, a therapeutically or prophylactically effective amount of a compound provided herein, or a pharmaceutically acceptable salt thereof, is about 5 mg. In some embodiments, a therapeutically or prophylactically effective amount of a compound provided herein, or a pharmaceutically acceptable salt thereof, is about 100 mg. In some embodiments, a therapeutically or prophylactically effective amount of a compound provided herein, or a pharmaceutically acceptable salt thereof, is about 150 mg. In some embodiments, a therapeutically or prophylactically effective amount of a compound provided herein, or a pharmaceutically acceptable salt thereof, is about 200 mg. In some embodiments, a therapeutically or prophylactically effective amount of a compound provided herein, or a pharmaceutically acceptable salt thereof, is about 250 mg. In some embodiments, a therapeutically or prophylactically effective amount of a compound provided herein, or a pharmaceutically acceptable salt thereof, is about 300 mg. In some embodiments, a therapeutically or prophylactically effective amount of a compound provided herein, or a pharmaceutically acceptable salt thereof, is about 350 mg. In some embodiments, a therapeutically or prophylactically effective amount of a compound provided herein, or a pharmaceutically acceptable salt thereof, is about 400 mg. In some embodiments, a therapeutically or prophylactically effective amount of a compound provided herein, or a pharmaceutically acceptable salt thereof, is about 450 mg. In some embodiments, a therapeutically or prophylactically effective amount of a compound provided herein, or a pharmaceutically acceptable salt thereof, is about 500 mg. In some embodiments, a therapeutically or prophylactically effective amount of a compound provided herein, or a pharmaceutically acceptable salt thereof, is about 550 mg. In some embodiments, a therapeutically or prophylactically effective amount of a compound provided herein, or a pharmaceutically acceptable salt thereof, is about 600 mg. In some embodiments, a therapeutically or prophylactically effective amount of a compound provided herein, or a pharmaceutically acceptable salt thereof, is about 650 mg. In some embodiments, a therapeutically or prophylactically effective amount of a compound provided herein, or a pharmaceutically acceptable salt thereof, is about 700 mg. In some embodiments, a therapeutically or prophylactically effective amount of a compound provided herein, or a pharmaceutically acceptable salt thereof, is about 750 mg. In some embodiments, a therapeutically or prophylactically effective amount of a compound provided herein, or a pharmaceutically acceptable salt thereof, is about 800 mg. In some embodiments, a therapeutically or prophylactically effective amount of a compound provided herein, or a pharmaceutically acceptable salt thereof, is about 850 mg. In some embodiments, a therapeutically or prophylactically effective amount of a compound provided herein, or a pharmaceutically acceptable salt thereof, is about 900 mg. In some embodiments, a therapeutically or prophylactically effective amount of a compound provided herein, or a pharmaceutically acceptable salt thereof, is about 950 mg. In some embodiments, a therapeutically or prophylactically effective amount of a compound provided herein, or a pharmaceutically acceptable salt thereof, is about 1000 mg. In some embodiments, a therapeutically or prophylactically effective amount of a compound provided herein, or a pharmaceutically acceptable salt thereof, is about 1050 mg. In some embodiments, a therapeutically or prophylactically effective amount of a compound provided herein, or a pharmaceutically acceptable salt thereof, is about 1100 mg. In some embodiments, a therapeutically or prophylactically effective amount of a compound provided herein, or a pharmaceutically acceptable salt thereof, is about 1150 mg. In some embodiments, a therapeutically or prophylactically effective amount of a compound provided herein, or a pharmaceutically acceptable salt thereof, is about 1200 mg. In some embodiments, a therapeutically or prophylactically effective amount of a compound provided herein, or a pharmaceutically acceptable salt thereof, is about 1250 mg. In some embodiments, a therapeutically or prophylactically effective amount of a compound provided herein, or a pharmaceutically acceptable salt thereof, is about 1300 mg. In some embodiments, a therapeutically or prophylactically effective amount of a compound provided herein, or a pharmaceutically acceptable salt thereof, is about 1350 mg. In some embodiments, a therapeutically or prophylactically effective amount of a compound provided herein, or a pharmaceutically acceptable salt thereof, is about 1400 mg. In some embodiments, a therapeutically or prophylactically effective amount of a compound provided herein, or a pharmaceutically acceptable salt thereof, is about 1450 mg. In some embodiments, a therapeutically or prophylactically effective amount of a compound provided herein, or a pharmaceutically acceptable salt thereof, is about 1500 mg. In some embodiments, a therapeutically or prophylactically effective amount of a compound provided herein, or a pharmaceutically acceptable salt thereof, is about 1550 mg. In some embodiments, a therapeutically or prophylactically effective amount of a compound provided herein, or a pharmaceutically acceptable salt thereof, is about 1600 mg. In some embodiments, a therapeutically or prophylactically effective amount of a compound provided herein, or a pharmaceutically acceptable salt thereof, is about 1650 mg. In some embodiments, a therapeutically or prophylactically effective amount of a compound provided herein, or a pharmaceutically acceptable salt thereof, is about 1700 mg. In some embodiments, a therapeutically or prophylactically effective amount of a compound provided herein, or a pharmaceutically acceptable salt thereof, is about 1750 mg. In some embodiments, a therapeutically or prophylactically effective amount of a compound provided herein, or a pharmaceutically acceptable salt thereof, is about 1800 mg. In some embodiments, a therapeutically or prophylactically effective amount of a compound provided herein, or a pharmaceutically acceptable salt thereof, is about 1850 mg. In some embodiments, a therapeutically or prophylactically effective amount of a compound provided herein, or a pharmaceutically acceptable salt thereof, is about 1900 mg. In some embodiments, a therapeutically or prophylactically effective amount of a compound provided herein, or a pharmaceutically acceptable salt thereof, is about 1950 mg. In some embodiments, a therapeutically or prophylactically effective amount of a compound provided herein, or a pharmaceutically acceptable salt thereof, is about 2000 mg. In some embodiments, a therapeutically or prophylactically effective amount of a compound provided herein, or a pharmaceutically acceptable salt thereof, is about 2100 mg. In some embodiments, a therapeutically or prophylactically effective amount of a compound provided herein, or a pharmaceutically acceptable salt thereof, is about 2200 mg. In some embodiments, a therapeutically or prophylactically effective amount of a compound provided herein, or a pharmaceutically acceptable salt thereof, is about 2300 mg. In some embodiments, a therapeutically or prophylactically effective amount of a compound provided herein, or a pharmaceutically acceptable salt thereof, is about 2400 mg.

A single dose of a compound, or a pharmaceutically acceptable salt thereof, or composition provided herein can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, 4, 5, 6, 7, or 8 weeks. In some embodiments, a single dose can be administered once or twice every four to eight weeks. In some embodiments, a single dose can be administered once or twice every four to seven weeks. In some embodiments, a single dose can be administered once or twice every four to six weeks. In some embodiments, a single dose can be administered once or twice every four to five weeks.

In some embodiments, one compound provided herein, such as a compound of Formula Ia or Ib, or a pharmaceutically acceptable salt thereof, may be administered within seconds, minutes, or hours of the administration of a second compound provided herein, such as a compound of Formula II, or a pharmaceutically acceptable salt thereof.

In some embodiments, a unit dose of a compound of Formula Ia or Ib, or a pharmaceutically acceptable salt thereof, is administered first, followed within seconds or minutes by administration of a unit dose of a compound of Formula II, or a pharmaceutically acceptable salt thereof. In some embodiments, a unit dose of a compound of Formula II, or a pharmaceutically acceptable salt thereof, is administered first, followed within seconds or minutes by administration of a unit dose of a compound of Formula Ia or Ib, or a pharmaceutically acceptable salt thereof.

In some embodiments, a unit dose of a compound of Formula Ia or Ib, or a pharmaceutically acceptable salt thereof, is administered first, followed within minutes or hours (e.g., about 1 to about 12 hours) by administration of a unit dose of a compound of Formula II, or a pharmaceutically acceptable salt thereof. In some embodiments, a unit dose of a compound of Formula II, or a pharmaceutically acceptable salt thereof, is administered first, followed within minutes or hours (e.g., about 1 to about 12 hours) by administration of a unit dose of a compound of Formula Ia or Ib, or a pharmaceutically acceptable salt thereof.

The methods provided herein may be applied to cell populations in vivo or ex vivo. “In vivo” means within a living individual, as within an animal or human. In this context, the methods provided herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. Exemplary tissue samples include tumors and biopsies thereof. In this context, the present disclosure may be used for a variety of purposes, including therapeutic and experimental purposes. For example, the present disclosure may be used ex vivo to determine the optimal schedule and/or dosing of administration of a compound as disclosed herein for a given cell type, individual, and other parameters. Information gleaned from such use may be used for experimental purposes or in the clinic to set protocols for in vivo treatment. Other ex vivo uses for which the present disclosure may be suited are described below or will become apparent to those skilled in the art. The selected compounds may be further characterized to examine the safety or tolerance dosage in human or non-human subjects. Such properties may be examined using commonly known methods to those skilled in the art.

The frequency of dosage of the compounds, salts, or compositions of the present disclosure will be determined by the needs of the individual patient and can be, for example, once per day, once per week, once per month, once per every two months, once per every three months, or once per every six months. Administration of the compounds, salts, or compositions of the present disclosure continues for as long as necessary to treat the Retroviridae infection, including an HIV infection, or any other indication described herein. For example, the compounds, salts, or compositions of the present disclosure, can be administered to a human suffering from a Retroviridae infection, including an HIV infection, for the duration of the human's life.

Administration can be intermittent, with a period of several or more days during which a patient receives a daily dose of the compounds, salts, or compositions of the present disclosure, followed by a period of several or more days during which a patient does not receive a daily dose of the compounds, salts, or compositions of the present disclosure. For example, a patient can receive a dose of the compounds, salts, or compositions, every other day, or three times per week. Again by way of example, a patient can receive a dose of the compounds, salts, or compositions each day for a period of from 1 to 14 days, followed by a period of 7 to 21 days during which the patient does not receive a dose of the compounds, salts, or compositions followed by a subsequent period (e.g., from 1 to 14 days) during which the patient again receives a daily dose of the compounds, salts, or compositions. Alternating periods of administration of the compounds, salts, or compositions followed by non-administration of the compounds, salts, or compositions can be repeated as clinically required to treat the patient.

The compounds of the present disclosure, or pharmaceutically acceptable salts thereof, or the pharmaceutical compositions of the present disclosure may be administered once, twice, three, or four times daily, using any suitable mode described above. Also, administration or treatment with the compounds, salts, or compositions may be continued for a number of days; for example, commonly treatment would continue for at least 7 days, 14 days, 28 days, 30 days, 31 days, 32 days, 33 days, 34 days, 35 days, 36 days, 37 days, 38 days, 39 days, 40 days, 41 days, 42 days, 43 days, 44 days, or 45 days, for one cycle of treatment. Treatment cycles are well known for Retroviridae infections, including an HIV infection. In some embodiments, treatment cycles are frequently alternated with resting periods of about 1 to 45 days, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 days between cycles. In some embodiments, the compounds are administered on the same day each month (e.g., on the first day of each month, the second day of each month, and the like). In some embodiments, the treatment cycles provided herein may be continuous.

In some embodiments, a compound or composition provided herein (e.g., a tablet comprising a compound of Formula Ia or Ib, or a pharmaceutically acceptable salt or free acid form thereof) is administered once every five to ten days, for example, once every five days, six days, seven days, eight days, nine days, or ten days. In some embodiments, a compound or composition provided herein (e.g., a tablet comprising a compound of Formula Ia or Ib, or a pharmaceutically acceptable salt or free acid form thereof) is administered once every six to eight days. In some embodiments, a compound or composition provided herein (e.g., a tablet comprising a compound of Formula Ia or Ib, or a pharmaceutically acceptable salt or free acid form thereof) is administered once every seven days (i.e., once-weekly).

In some embodiments, a compound or composition provided herein (e.g., a tablet comprising a compound of Formula Ia or Ib, or a pharmaceutically acceptable salt or free acid form thereof) is orally administered once every five to ten days, for example, once every five days, six days, seven days, eight days, nine days, or ten days. In some embodiments, a compound or composition provided herein (e.g., a tablet comprising a compound of Formula Ia or Ib, or a pharmaceutically acceptable salt or free acid form thereof) is orally administered once every six to eight days. In some embodiments, a compound or composition provided herein (e.g., a tablet comprising a compound of Formula Ia or Ib, or a pharmaceutically acceptable salt or free acid form thereof) is orally administered once every seven days (i.e., once-weekly).

In some embodiments, a compound or composition provided herein (e.g., a tablet comprising a compound of Formula Ia or Ib, or a pharmaceutically acceptable salt or free acid form thereof) is orally administered once every month.

In some embodiments, a compound provided herein, or a pharmaceutically acceptable salt thereof, is administered orally in a single dose of about 100 mg once or twice monthly. In some embodiments, a compound provided herein, or a pharmaceutically acceptable salt thereof, is administered orally in a single dose of about 200 mg once or twice monthly. In some embodiments, a compound provided herein, or a pharmaceutically acceptable salt thereof, is administered orally in a single dose of about 300 mg once or twice monthly. In some embodiments, a compound provided herein, or a pharmaceutically acceptable salt thereof, is administered orally in a single dose of about 350 mg once or twice monthly. In some embodiments, a compound provided herein, or a pharmaceutically acceptable salt thereof, is administered orally in a single dose of about 400 mg once or twice monthly. In some embodiments, a compound provided herein, or a pharmaceutically acceptable salt thereof, is administered orally in a single dose of about 450 mg once or twice monthly. In some embodiments, a compound provided herein, or a pharmaceutically acceptable salt thereof, is administered orally in a single dose of about 500 once or twice monthly. In some embodiments, a compound provided herein, or a pharmaceutically acceptable salt thereof, is administered orally in a single dose of about 550 mg once or twice monthly. In some embodiments, a compound provided herein, or a pharmaceutically acceptable salt thereof, is administered orally in a single dose of about 600 mg once or twice monthly. In some embodiments, a compound provided herein, or a pharmaceutically acceptable salt thereof, is administered orally in a single dose of about 650 mg once or twice monthly. In some embodiments, a compound provided herein, or a pharmaceutically acceptable salt thereof, is administered orally in a single dose of about 700 mg once or twice monthly. In some embodiments, a compound provided herein, or a pharmaceutically acceptable salt thereof, is administered orally in a single dose of about 750 mg once or twice monthly. In some embodiments, a compound provided herein, or a pharmaceutically acceptable salt thereof, is administered orally in a single dose of about 800 mg once or twice monthly. In some embodiments, a compound provided herein, or a pharmaceutically acceptable salt thereof, is administered orally in a single dose of about 850 mg once or twice monthly. In some embodiments, a compound provided herein, or a pharmaceutically acceptable salt thereof, is administered orally in a single dose of about 900 mg once or twice monthly. In some embodiments, a compound provided herein, or a pharmaceutically acceptable salt thereof, is administered orally in a single dose of about 950 mg once or twice monthly. In some embodiments, a compound provided herein, or a pharmaceutically acceptable salt thereof, is administered orally in a single dose of about 1000 mg once or twice monthly. In some embodiments, a compound provided herein, or a pharmaceutically acceptable salt thereof, is administered orally in a single dose of about 1050 mg once or twice monthly. In some embodiments, a compound provided herein, or a pharmaceutically acceptable salt thereof, is administered orally in a single dose of about 1100 mg once or twice monthly. In some embodiments, a compound provided herein, or a pharmaceutically acceptable salt thereof, is administered orally in a single dose of about 1150 mg once or twice monthly. In some embodiments, a compound provided herein, or a pharmaceutically acceptable salt thereof, is administered orally in a single dose of about 1200 mg once or twice monthly. In some embodiments, a compound provided herein, or a pharmaceutically acceptable salt thereof, is administered orally in a single dose of about 1250 mg once or twice monthly. In some embodiments, a compound provided herein, or a pharmaceutically acceptable salt thereof, is administered orally in a single dose of about 1300 mg once or twice monthly. In some embodiments, a compound provided herein, or a pharmaceutically acceptable salt thereof, is administered orally in a single dose of about 1350 mg once or twice monthly. In some embodiments, a compound provided herein, or a pharmaceutically acceptable salt thereof, is administered orally in a single dose of about 1400 mg once or twice monthly. In some embodiments, a compound provided herein, or a pharmaceutically acceptable salt thereof, is administered orally in a single dose of about 1450 mg once or twice monthly. In some embodiments, a compound provided herein, or a pharmaceutically acceptable salt thereof, is administered orally in a single dose of about 1500 mg once or twice monthly. In some embodiments, a compound provided herein, or a pharmaceutically acceptable salt thereof, is administered orally in a single dose of about 1550 mg once or twice monthly. In some embodiments, a compound provided herein, or a pharmaceutically acceptable salt thereof, is administered orally in a single dose of about 1600 mg once or twice monthly. In some embodiments, a compound provided herein, or a pharmaceutically acceptable salt thereof, is administered orally in a single dose of about 1650 mg once or twice monthly. In some embodiments, a compound provided herein, or a pharmaceutically acceptable salt thereof, is administered orally in a single dose of about 1700 mg once or twice monthly. In some embodiments, a compound provided herein, or a pharmaceutically acceptable salt thereof, is administered orally in a single dose of about 1750 mg once or twice monthly. In some embodiments, a compound provided herein, or a pharmaceutically acceptable salt thereof, is administered orally in a single dose of about 1800 mg once or twice monthly. In some embodiments, a compound provided herein, or a pharmaceutically acceptable salt thereof, is administered orally in a single dose of about 1850 mg once or twice monthly. In some embodiments, a compound provided herein, or a pharmaceutically acceptable salt thereof, is administered orally in a single dose of about 1900 mg once or twice monthly. In some embodiments, a compound provided herein, or a pharmaceutically acceptable salt thereof, is administered orally in a single dose of about 1950 mg once or twice monthly. In some embodiments, a compound provided herein, or a pharmaceutically acceptable salt thereof, is administered orally in a single dose of about 2000 mg once or twice monthly. In some embodiments, a compound provided herein, or a pharmaceutically acceptable salt thereof, is administered orally in a single dose of about 2100 mg once or twice monthly. In some embodiments, a compound provided herein, or a pharmaceutically acceptable salt thereof, is administered orally in a single dose of about 2200 mg once or twice monthly. In some embodiments, a compound provided herein, or a pharmaceutically acceptable salt thereof, is administered orally in a single dose of about 2300 mg once or twice monthly. In some embodiments, a compound provided herein, or a pharmaceutically acceptable salt thereof, is administered orally in a single dose of about 2400 mg once or twice monthly.

In some embodiments, the single dose described herein is administered once every month. In some embodiments, the single dose described herein is administered twice every month.

In some embodiments, a single dose can be administered once or twice every four weeks. In some embodiments, a single dose can be administered once or twice every five weeks. In some embodiments, a single dose can be administered once or twice every six weeks. In some embodiments, a single dose can be administered once or twice every seven weeks. In some embodiments, a single dose can be administered once or twice every eight weeks.

In some embodiments, a single dose can be administered once or twice every 30 days. In some embodiments, a single dose can be administered once or twice every 31 days. In some embodiments, a single dose can be administered once or twice every 32 days. In some embodiments, a single dose can be administered once or twice every 33 days. In some embodiments, a single dose can be administered once or twice every 34 days. In some embodiments, a single dose can be administered once or twice every 35 days. In some embodiments, a single dose can be administered once or twice every 36 days. In some embodiments, a single dose can be administered once or twice every 37 days. In some embodiments, a single dose can be administered once or twice every 38 days. In some embodiments, a single dose can be administered once or twice every 39 days. In some embodiments, a single dose can be administered once or twice every 40 days. In some embodiments, a single dose can be administered once or twice every 41 days. In some embodiments, a single dose can be administered once or twice every 42 days. In some embodiments, a single dose can be administered once or twice every 43 days. In some embodiments, a single dose can be administered once or twice every 44 days. In some embodiments, a single dose can be administered once or twice every 45 days.

In some embodiments, the compounds provided herein are administered on a split dosing schedule (e.g., a compound of Formula Ia or Ib and the PGP inhibitor are administered in the morning and a compound of Formula Ia or Ib and/or the PGP inhibitor is administered in the evening).

In some embodiments, a compound provided herein (e.g., a compound of Formula Ia or Ib) and the PGP inhibitor provided herein (e.g., encequidar) are administered to the patient in the morning and in the evening.

In some embodiments, a compound provided herein (e.g., a compound of Formula Ia or Ib) and the PGP inhibitor provided herein (e.g., encequidar) are administered to the patient in the morning and a compound provided herein (e.g., a compound of Formula Ia or Ib) is administered to the patient in the evening.

In some embodiments, a compound provided herein (e.g., a compound of Formula Ia or Ib) and the PGP inhibitor provided herein (e.g., encequidar) are administered to the patient in the morning and the PGP inhibitor provided herein (e.g., encequidar) is administered to the patient in the evening.

In the regimens disclosed herein that involve administration in the morning and the evening, the morning and the evening may be between 8 hours apart and 16 hours apart, such as between 10 hours apart and 14 hours apart, or 12 hours apart. In some embodiments, a compound provided herein (e.g., a compound of Formula Ia or Ib) and the PGP inhibitor provided herein (e.g., encequidar) are administered to the patient in the morning and in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a compound provided herein (e.g., a compound of Formula Ia or Ib) and the PGP inhibitor provided herein (e.g., encequidar) are administered to the patient in the morning and a compound provided herein (e.g., a compound of Formula Ia or Ib) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a compound provided herein (e.g., a compound of Formula Ia or Ib) and the PGP inhibitor provided herein (e.g., encequidar) are administered to the patient in the morning and the PGP inhibitor provided herein (e.g., encequidar) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a compound provided herein (e.g., a compound of Formula Ia or Ib) and the PGP inhibitor (e.g., encequidar) are administered to the patient in the morning and in the evening, once per month.

In some embodiments, a compound provided herein (e.g., a compound of Formula Ia or Ib) and the PGP inhibitor (e.g., encequidar) are administered in the morning and a compound provided herein (e.g., a compound of Formula Ia or Ib) is administered to the patient in the evening, once per month.

In some embodiments, a compound provided herein (e.g., a compound of Formula Ia or Ib) and the PGP inhibitor (e.g., encequidar) are administered in the morning and the PGP inhibitor (e.g., encequidar) is administered to the patient in the evening, once per month.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and in the evening.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition provided herein (e.g., a tablet comprising a compound of Formula Ia or Ib) is administered to the patient in the evening.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition comprising the PGP inhibitor provided herein (e.g., a solution or suspension comprising a compound of Formula II) is administered to the patient in the evening.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition provided herein (e.g., a tablet comprising a compound of Formula Ia or Ib) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition comprising the PGP inhibitor provided herein (e.g., a solution or suspension comprising a compound of Formula II) is administered to the patient in the evening, once every 28 days, once every 29 days, once every 30 days, once every 31 days, once every 32 days, once every 33 days, once every 34 days, once every 35 days, once every 36 days, once every 37 days, once every 38 days, once every 39 days, once every 40 days, once every 41 days, once every 42 days, once every 43 days, once every 44 days, or once every 45 days.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and in the evening, once per month.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition provided herein (e.g., a tablet comprising a compound of Formula Ia or Ib) is administered to the patient in the evening, once per month.

In some embodiments, a pharmaceutical composition provided herein (e.g., a tablet comprising a compound of Formula Ia or Ib) and a pharmaceutical composition comprising the PGP inhibitor provided herein (e.g., a solution or suspension comprising a compound of Formula II) are administered to the patient in the morning and a pharmaceutical composition comprising the PGP inhibitor provided herein (e.g., a solution or suspension comprising a compound of Formula II) is administered to the patient in the evening, once per month.

### Pharmaceutical Compositions of Compounds of Formula Ia and Formula Ib (Compound 2)

In some embodiments, the pharmaceutical composition (i.e. formulation) comprising the compound of Formula Ia or Ib, or a pharmaceutically acceptable salt thereof, is suitable for oral administration. In some embodiments, the formulation comprising the compound of Formula Ia or Ib, or a pharmaceutically acceptable salt thereof, is a tablet. In some embodiments, the formulation comprising the compound of Formula Ia or Ib, or a pharmaceutically acceptable salt thereof, is a tablet suitable for oral administration.

The pharmaceutically acceptable excipients of the tablets disclosed herein should be compatible with the other ingredients of the formulation and physiologically innocuous to the recipient thereof. Examples of suitable excipients are well known to the person skilled in the art of tablet formulation and may be found e.g. in Handbook of Pharmaceutical Excipients (eds. Rowe, Sheskey & Quinn), 6th edition 2009. As used herein the term “excipients” is intended to refer to inter alia basifying agents, solubilisers, glidants, fillers, binders, lubricant, diluents, preservatives, surface active agents, dispersing agents and the like. The term also includes agents such as sweetening agents, flavoring agents, coloring agents and preserving agents. Such components will generally be present in admixture within the tablet.

Examples of solubilisers include, but are not limited to, surfactants (including both ionic and non-ionic surfactants) such as sodium lauryl sulphate, cetyltrimethylammonium bromide, polysorbates (such as polysorbate 20 or 80), poloxamers (such as poloxamer 188 or 207), and macrogols. Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc.

Examples of disintegrants include, but are not limited to, starches, celluloses, cross-linked PVP, sodium starch glycolate, croscarmellose sodium, and the like. Examples of fillers (also known as bulking agents or diluents) include, but are not limited to, starches, maltodextrins, polyols (such as lactose), and celluloses. Examples of binders include, but are not limited to, cross-linked PVP, HPMC, microcrystalline cellulose, sucrose, starches, and the like.

In some embodiments, the tablet disclosed herein comprise a compound of Formula Ia or Ib, or a pharmaceutically acceptable salt thereof, and one or more pharmaceutically acceptable excipients selected from the group consisting of a diluent, a disintegrant, and a lubricant.

In some embodiments, the tablet provided herein comprises about 30 w/w % to about 50 w/w % of the compound of Formula Ia or Ib, or a pharmaceutically acceptable salt thereof, for example, about 30 w/w %, about 35 w/w %, about 40 w/w %, about 45 w/w %, or about 50 w/w %. In some embodiments, the tablet provided herein comprises about 30 w/w % to about 50 w/w % of a free acid form of the compound of Formula Ia or Ib. In some embodiments, the tablet provided herein comprises about 30 w/w % to about 50 w/w % of a free acid form of the compound of Formula Ia. In some embodiments, the tablet provided herein comprises about 30 w/w % to about 50 w/w % of a free acid form of the compound of Formula Ib. In some embodiments, the tablet provided herein comprises about 30 w/w % to about 50 w/w % of a crystalline form of the compound of Formula Ib. In some embodiments, the tablet provided herein comprises about 30 w/w % to about 50 w/w % of the compound of Formula Ib, crystalline Form I.

In some embodiments, the tablet comprises about 35 w/w % to about 45 w/w % of the compound of Formula Ia or Ib, or a pharmaceutically acceptable salt thereof. In some embodiments, the tablet comprises about 35 w/w % to about 45 w/w % of the compound of Formula Ia, or a pharmaceutically acceptable salt thereof. In some embodiments, the tablet comprises about 35 w/w % to about 45 w/w % of the compound of Formula Ia. In some embodiments, the tablet comprises about 35 w/w % to about 45 w/w % of the compound of Formula Ib, or a pharmaceutically acceptable salt thereof. In some embodiments, the tablet comprises about 35 w/w % to about 45 w/w % of a free acid form of the compound of Formula Ib. In some embodiments, the tablet comprises about 35 w/w % to about 45 w/w % of a crystalline form of the compound of Formula Ib. In some embodiments, the tablet comprises about 35 w/w % to about 45 w/w % of the compound of Formula Ib, crystalline Form I.

In some embodiments, the tablet comprises about 40 w/w % of the compound of Formula Ia or Ib, or a pharmaceutically acceptable salt thereof. In some embodiments, the tablet comprises about 40 w/w % of the compound of Formula Ia, or a pharmaceutically acceptable salt thereof. In some embodiments, the tablet comprises about 40 w/w % of a free acid form of the compound of Formula Ia. In some embodiments, the tablet comprises about 40 w/w % of the compound of Formula Ib, or a pharmaceutically acceptable salt thereof. In some embodiments, the tablet comprises about 40 w/w % of a free acid form of the compound of Formula Ib. In some embodiments, the tablet comprises about 40 w/w % of a crystalline form of the compound of Formula Ib. In some embodiments, the tablet comprises about 40 w/w % of the compound of Formula Ib, crystalline Form I.

In some embodiments, the tablet comprises about 25 w/w % to about 35 w/w % of mannitol, for example, about 25 w/w %, about 26 w/w %, about 27 w/w %, about 28 w/w %, about 29 w/w %, about 30 w/w %, about 31 w/w %, about 32 w/w %, about 33 w/w %, about 34 w/w %, or about 35 w/w % mannitol.

In some embodiments, the tablet comprises about 25 w/w % to about 30 w/w % of mannitol. In some embodiments, the tablet comprises about 27 w/w % of mannitol.

In some embodiments, the tablet comprises about 25 w/w % to about 35 w/w % of microcrystalline cellulose, for example, about 25 w/w %, about 26 w/w %, about 27 w/w %, about 28 w/w %, about 29 w/w %, about 30 w/w %, about 31 w/w %, about 32 w/w %, about 33 w/w %, about 34 w/w %, or about 35 w/w % microcrystalline cellulose. In some embodiments, the tablet comprises about 25 w/w % to about 30 w/w % of microcrystalline cellulose. In some embodiments, the tablet comprises about 27.5 w/w % of microcrystalline cellulose.

In some embodiments, the tablet comprises about 1 w/w % to about 10 w/w % of crospovidone, for example, about 1%, about 2%, about 3%, about 4%, about 5%, about 6%, about 7%, about 8%, about 9%, or about 10% crospovidone. In some embodiments, the tablet comprises about 2 w/w % to about 6 w/w % of crospovidone. In some embodiments, the tablet comprises about 4 w/w % of crospovidone.

In some embodiments, the tablet comprises about 1 w/w % to about 5 w/w % of magnesium stearate, for example, about 1%, about 2%, about 3%, about 4%, or about 5% magnesium stearate. In some embodiments, the tablet comprises about 1 w/w % to about 3 w/w % of magnesium stearate. In some embodiments, the tablet comprises about 1.5 w/w % of magnesium stearate.

In some embodiments, the tablet comprises the compound of Formula Ia or Ib, or a pharmaceutically acceptable salt thereof, mannitol, microcrystalline cellulose, crospovidone, and magnesium stearate. In some embodiments, the tablet comprises the free acid form of the compound of Formula Ia or Ib, mannitol, microcrystalline cellulose, crospovidone, and magnesium stearate. In some embodiments, the tablet comprises an amorphous form of the free acid form of the compound of Formula Ib, mannitol, microcrystalline cellulose, crospovidone, and magnesium stearate.

In some embodiments, the tablet consists essentially of the compound of Formula Ia or Ib, or a pharmaceutically acceptable salt thereof, mannitol, microcrystalline cellulose, crospovidone, and magnesium stearate. In some embodiments, the tablet consists essentially of the free acid form of the compound of Formula Ia or Ib, mannitol, microcrystalline cellulose, crospovidone, and magnesium stearate. In some embodiments, the tablet consists essentially of an amorphous form of the free acid form of the compound of Formula Ib, mannitol, microcrystalline cellulose, crospovidone, and magnesium stearate.

In some embodiments, the tablet provided herein comprises or consists essentially of:
about 30 w/w % to about 50 w/w % of the compound of Formula Ia or Ib, or a pharmaceutically acceptable salt thereof;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises or consists essentially of:
about 35 w/w % to about 45 w/w % of the compound of Formula Ia or Ib, or a pharmaceutically acceptable salt thereof;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises or consists essentially of:
about 40 w/w % of the compound of Formula Ia or Ib, or a pharmaceutically acceptable salt thereof;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises or consists essentially of:
about 30 w/w % to about 50 w/w % of the compound of Formula Ia, or a pharmaceutically acceptable salt thereof;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises or consists essentially of:
about 35 w/w % to about 45 w/w % of the compound of Formula Ia, or a pharmaceutically acceptable salt thereof;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises or consists essentially of:
about 40 w/w % of the compound of Formula Ia, or a pharmaceutically acceptable salt thereof;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises or consists essentially of:
about 30 w/w % to about 50 w/w % of the compound of Formula Ia (i.e., the free acid form of the compound of Formula Ia);
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises or consists essentially of:
about 35 w/w % to about 45 w/w % of the compound of Formula Ia;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises or consists essentially of:
about 40 w/w % of the compound of Formula Ia;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises or consists essentially of:
about 30 w/w % to about 50 w/w % of the compound of Formula Ib, or a pharmaceutically acceptable salt thereof;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises or consists essentially of:
about 35 w/w % to about 45 w/w % of the compound of Formula Ib, or a pharmaceutically acceptable salt thereof;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises or consists essentially of:
about 40 w/w % of the compound of Formula Ib, or a pharmaceutically acceptable salt thereof;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises or consists essentially of:
about 30 w/w % to about 50 w/w % of the compound of Formula Ib (i.e., the free acid form of the compound of Formula Ib);
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises or consists essentially of:
about 35 w/w % to about 45 w/w % of the compound of Formula Ib;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises or consists essentially of:
about 40 w/w % of the compound of Formula Ib;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises or consists essentially of:
about 30 w/w % to about 50 w/w % of a crystalline form of the compound of Formula Ib (i.e., a crystalline form of the free acid form of the compound of Formula Ib);
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises or consists essentially of:
about 35 w/w % to about 45 w/w % of a crystalline form of the compound of Formula Ib;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises or consists essentially of:
about 40 w/w % of a crystalline form of the compound of Formula Ib;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises or consists essentially of:
about 30 w/w % to about 50 w/w % of the compound of Formula Ib, crystalline Form I;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises or consists essentially of:
about 35 w/w % to about 45 w/w % of the compound of Formula Ib, crystalline Form I;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises or consists essentially of:
about 40 w/w % of the compound of Formula Ib, crystalline Form I;
about 27 w/w % of mannitol;
about 27.5 w/w % of microcrystalline cellulose;
about 4 w/w % of crospovidone; and
about 1.5 w/w % of magnesium stearate.

In some embodiments, the tablet comprises about 1 mg to about 2400 mg of the compound of Formula Ia or Ib, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2100 mg, about 2200 mg, about 2300 mg, or about 2400 mg of the compound of Formula Ia or Ib, or a pharmaceutically acceptable salt thereof.

In some embodiments, the tablet comprises about 5 mg to about 1000 mg of the compound of Formula Ia or Ib, or a pharmaceutically acceptable salt thereof. In some embodiments, the tablet comprises about 5 mg to about 750 mg of the compound of Formula Ia or Ib, or a pharmaceutically acceptable salt thereof. In some embodiments, the tablet comprises about 5 mg to about 500 mg of the compound of Formula Ia or Ib, or a pharmaceutically acceptable salt thereof. In some embodiments, the tablet comprises about 50 mg to about 450 mg of the compound of Formula Ia or Ib, or a pharmaceutically acceptable salt thereof. In some embodiments, the tablet comprises about 75 mg to about 425 mg of the compound of Formula Ia or Ib, or a pharmaceutically acceptable salt thereof.

In some embodiments, the tablet comprises about 75 mg to about 125 mg of the compound of Formula Ia or Ib, or a pharmaceutically acceptable salt thereof. In some embodiments, the tablet comprises about 100 mg of the compound of Formula Ia or Ib, or a pharmaceutically acceptable salt thereof.

In some embodiments, the tablet comprises about 175 mg to about 225 mg of the compound of Formula Ia or Ib, or a pharmaceutically acceptable salt thereof. In some embodiments, the tablet comprises about 200 mg of the compound of Formula Ia or Ib, or a pharmaceutically acceptable salt thereof.

In some embodiments, the tablet comprises about 275 mg to about 325 mg of the compound of Formula Ia or Ib, or a pharmaceutically acceptable salt thereof. In some embodiments, the tablet comprises about 300 mg of the compound of Formula Ia or Ib, or a pharmaceutically acceptable salt thereof.

In some embodiments, the tablet comprises about 375 mg to about 425 mg of the compound of Formula Ia or Ib, or a pharmaceutically acceptable salt thereof. In some embodiments, the tablet comprises about 400 mg of the compound of Formula Ia or Ib, or a pharmaceutically acceptable salt thereof.

In some embodiments, the tablet comprises about 1 mg to about 500 mg of the compound of Formula Ia, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound of Formula Ia, or a pharmaceutically acceptable salt thereof.

In some embodiments, the tablet comprises about 5 mg to about 500 mg of the compound of Formula Ia, or a pharmaceutically acceptable salt thereof. In some embodiments, the tablet comprises about 50 mg to about 450 mg of the compound of Formula Ia, or a pharmaceutically acceptable salt thereof. In some embodiments, the tablet comprises about 75 mg to about 425 mg of the compound of Formula Ia, or a pharmaceutically acceptable salt thereof.

In some embodiments, the tablet comprises about 75 mg to about 125 mg of the compound of Formula Ia, or a pharmaceutically acceptable salt thereof. In some embodiments, the tablet comprises about 100 mg of the compound of Formula Ia, or a pharmaceutically acceptable salt thereof.

In some embodiments, the tablet comprises about 175 mg to about 225 mg of the compound of Formula Ia, or a pharmaceutically acceptable salt thereof. In some embodiments, the tablet comprises about 200 mg of the compound of Formula Ia, or a pharmaceutically acceptable salt thereof.

In some embodiments, the tablet comprises about 275 mg to about 325 mg of the compound of Formula Ia, or a pharmaceutically acceptable salt thereof. In some embodiments, the tablet comprises about 300 mg of the compound of Formula Ia, or a pharmaceutically acceptable salt thereof.

In some embodiments, the tablet comprises about 375 mg to about 425 mg of the compound of Formula Ia, or a pharmaceutically acceptable salt thereof. In some embodiments, the tablet comprises about 400 mg of the compound of Formula Ia, or a pharmaceutically acceptable salt thereof.

In some embodiments, the tablet comprises about 1 mg to about 500 mg of the compound of Formula Ib, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound of Formula Ib, or a pharmaceutically acceptable salt thereof.

In some embodiments, the tablet comprises about 5 mg to about 500 mg of the compound of Formula Ib, or a pharmaceutically acceptable salt thereof. In some embodiments, the tablet comprises about 50 mg to about 450 mg of the compound of Formula Ib, or a pharmaceutically acceptable salt thereof. In some embodiments, the tablet comprises about 75 mg to about 425 mg of the compound of Formula Ib, or a pharmaceutically acceptable salt thereof.

In some embodiments, the tablet comprises about 75 mg to about 125 mg of the compound of Formula Ib, or a pharmaceutically acceptable salt thereof. In some embodiments, the tablet comprises about 100 mg of the compound of Formula Ib, or a pharmaceutically acceptable salt thereof.

In some embodiments, the tablet comprises about 175 mg to about 225 mg of the compound of Formula Ib, or a pharmaceutically acceptable salt thereof. In some embodiments, the tablet comprises about 200 mg of the compound of Formula Ib, or a pharmaceutically acceptable salt thereof.

In some embodiments, the tablet comprises about 275 mg to about 325 mg of the compound of Formula Ib, or a pharmaceutically acceptable salt thereof. In some embodiments, the tablet comprises about 300 mg of the compound of Formula Ib, or a pharmaceutically acceptable salt thereof.

In some embodiments, the tablet comprises about 375 mg to about 425 mg of the compound of Formula Ib, or a pharmaceutically acceptable salt thereof. In some embodiments, the tablet comprises about 400 mg of the compound of Formula Ib, or a pharmaceutically acceptable salt thereof.

In some embodiments, the tablet comprises about 1 mg to about 500 mg of a crystalline form of the compound of Formula Ib, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of a crystalline form of the compound of Formula Ib.

In some embodiments, the tablet comprises about 5 mg to about 500 mg of a crystalline form of the compound of Formula Ib. In some embodiments, the tablet comprises about 50 mg to about 450 mg of a crystalline form of the compound of Formula Ib. In some embodiments, the tablet comprises about 75 mg to about 425 mg of a crystalline form of the compound of Formula Ib.

In some embodiments, the tablet comprises about 75 mg to about 125 mg of a crystalline form of the compound of Formula Ib. In some embodiments, the tablet comprises about 100 mg of a crystalline form of the compound of Formula Ib.

In some embodiments, the tablet comprises about 175 mg to about 225 mg of a crystalline form of the compound of Formula Ib. In some embodiments, the tablet comprises about 200 mg of a crystalline form of the compound of Formula Ib.

In some embodiments, the tablet comprises about 275 mg to about 325 mg of a crystalline form of the compound of Formula Ib. In some embodiments, the tablet comprises about 300 mg of a crystalline form of the compound of Formula Ib.

In some embodiments, the tablet comprises about 375 mg to about 425 mg of a crystalline form of the compound of Formula Ib. In some embodiments, the tablet comprises about 400 mg of a crystalline form of the compound of Formula Ib.

In some embodiments, the tablet comprises about 1 mg to about 500 mg of the compound of Formula Ib, crystalline Form I, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound of Formula Ib, crystalline Form I.

In some embodiments, the tablet comprises about 5 mg to about 500 mg of the compound of Formula Ib, crystalline Form I. In some embodiments, the tablet comprises about 50 mg to about 450 mg of the compound of Formula Ib, crystalline Form I. In some embodiments, the tablet comprises about 75 mg to about 425 mg of the compound of Formula Ib, crystalline Form I.

In some embodiments, the tablet comprises about 75 mg to about 125 mg of the compound of Formula Ib, crystalline Form I. In some embodiments, the tablet comprises about 100 mg of the compound of Formula Ib, crystalline Form I.

In some embodiments, the tablet comprises about 175 mg to about 225 mg of the compound of Formula Ib, crystalline Form I. In some embodiments, the tablet comprises about 200 mg of the compound of Formula Ib, crystalline Form I.

In some embodiments, the tablet comprises about 275 mg to about 325 mg of the compound of Formula Ib, crystalline Form I. In some embodiments, the tablet comprises about 300 mg of the compound of Formula Ib, crystalline Form I.

In some embodiments, the tablet comprises about 375 mg to about 425 mg of the compound of Formula Ib, crystalline Form I. In some embodiments, the tablet comprises about 400 mg of the compound of Formula Ib, crystalline Form I.

In some embodiments, the method comprises orally administering about 5 mg to about 500 mg of the compound of Formula Ia or Ib, or a pharmaceutically acceptable salt thereof to the patient, once every seven days. In some embodiments, the method comprises orally administering about 50 mg to about 450 mg of the compound of Formula Ia or Ib, or a pharmaceutically acceptable salt thereof to the patient, once every seven days. In some embodiments, the method comprises orally administering about 75 mg to about 425 mg of the compound of Formula Ia or Ib, or a pharmaceutically acceptable salt thereof to the patient, once every seven days.

In some embodiments, the method comprises orally administering about 75 mg to about 125 mg of the compound of Formula Ia or Ib, or a pharmaceutically acceptable salt thereof to the patient, once every seven days. In some embodiments, the method comprises orally administering about 100 mg of the compound of Formula Ia or Ib, or a pharmaceutically acceptable salt thereof to the patient, once every seven days.

In some embodiments, the method comprises orally administering about 175 mg to about 225 mg of the compound of Formula Ia or Ib, or a pharmaceutically acceptable salt thereof to the patient, once every seven days. In some embodiments, the method comprises orally administering about 200 mg of the compound of Formula Ia or Ib, or a pharmaceutically acceptable salt thereof to the patient, once every seven days.

In some embodiments, the method comprises orally administering about 275 mg to about 325 mg of the compound of Formula Ia or Ib, or a pharmaceutically acceptable salt thereof to the patient, once every seven days. In some embodiments, the method comprises orally administering about 300 mg of the compound of Formula Ia or Ib, or a pharmaceutically acceptable salt thereof to the patient, once every seven days.

In some embodiments, the method comprises orally administering about 375 mg to about 425 mg of the compound of Formula Ia or Ib, or a pharmaceutically acceptable salt thereof to the patient, once every seven days. In some embodiments, the method comprises orally administering about 400 mg of the compound of Formula Ia or Ib, or a pharmaceutically acceptable salt thereof to the patient, once every seven days.

In some embodiments, the method comprises orally administering about 5 mg to about 500 mg of the compound of Formula Ia or Ib to the patient, once every seven days. In some embodiments, the method comprises orally administering about 50 mg to about 450 mg of the compound of Formula Ia or Ib to the patient, once every seven days. In some embodiments, the method comprises orally administering about 75 mg to about 425 mg of the compound of Formula Ia or Ib to the patient, once every seven days.

In some embodiments, the method comprises orally administering about 75 mg to about 125 mg of the compound of Formula Ia or Ib to the patient, once every seven days. In some embodiments, the method comprises orally administering about 100 mg of the compound of

Formula Ia or Ib to the patient, once every seven days.

In some embodiments, the method comprises orally administering about 175 mg to about 225 mg of the compound of Formula Ia or Ib to the patient, once every seven days. In some embodiments, the method comprises orally administering about 200 mg of the compound of Formula Ia or Ib to the patient, once every seven days.

In some embodiments, the method comprises orally administering about 275 mg to about 325 mg of the compound of Formula Ia or Ib to the patient, once every seven days. In some embodiments, the method comprises orally administering about 300 mg of the compound of Formula Ia or Ib to the patient, once every seven days.

In some embodiments, the method comprises orally administering about 375 mg to about 425 mg of the compound of Formula Ia or Ib to the patient, once every seven days. In some embodiments, the method comprises orally administering about 400 mg of the compound of Formula Ia or Ib to the patient, once every seven days.

In some embodiments, the method comprises orally administering about 5 mg to about 500 mg of the compound of Formula Ia to the patient, once every seven days. In some embodiments, the method comprises orally administering about 50 mg to about 450 mg of the compound of Formula Ia to the patient, once every seven days. In some embodiments, the method comprises orally administering about 75 mg to about 425 mg of the compound of Formula Ia to the patient, once every seven days.

In some embodiments, the method comprises orally administering about 75 mg to about 125 mg of the compound of Formula Ia to the patient, once every seven days. In some embodiments, the method comprises orally administering about 100 mg of the compound of Formula Ia to the patient, once every seven days.

In some embodiments, the method comprises orally administering about 175 mg to about 225 mg of the compound of Formula Ia to the patient, once every seven days. In some embodiments, the method comprises orally administering about 200 mg of the compound of Formula Ia to the patient, once every seven days.

In some embodiments, the method comprises orally administering about 275 mg to about 325 mg of the compound of Formula Ia to the patient, once every seven days. In some embodiments, the method comprises orally administering about 300 mg of the compound of Formula Ia to the patient, once every seven days.

In some embodiments, the method comprises orally administering about 375 mg to about 425 mg of the compound of Formula Ia to the patient, once every seven days. In some embodiments, the method comprises orally administering about 400 mg of the compound of Formula Ia to the patient, once every seven days.

In some embodiments, the method comprises orally administering about 5 mg to about 500 mg of the compound of Formula Ib to the patient, once every seven days. In some embodiments, the method comprises orally administering about 50 mg to about 450 mg of the compound of Formula Ib to the patient, once every seven days. In some embodiments, the method comprises orally administering about 75 mg to about 425 mg of the compound of Formula Ib to the patient, once every seven days.

In some embodiments, the method comprises orally administering about 75 mg to about 125 mg of the compound of Formula Ib to the patient, once every seven days. In some embodiments, the method comprises orally administering about 100 mg of the compound of Formula Ib to the patient, once every seven days.

In some embodiments, the method comprises orally administering about 175 mg to about 225 mg of the compound of Formula Ib to the patient, once every seven days. In some embodiments, the method comprises orally administering about 200 mg of the compound of Formula Ib to the patient, once every seven days.

In some embodiments, the method comprises orally administering about 275 mg to about 325 mg of the compound of Formula Ib to the patient, once every seven days. In some embodiments, the method comprises orally administering about 300 mg of the compound of Formula Ib to the patient, once every seven days.

In some embodiments, the method comprises orally administering about 375 mg to about 425 mg of the compound of Formula Ib to the patient, once every seven days. In some embodiments, the method comprises orally administering about 400 mg of the compound of Formula Ib to the patient, once every seven days.

In some embodiments, the method comprises orally administering about 5 mg to about 500 mg of a crystalline form of the compound of Formula Ib (e.g., crystalline Form I) to the patient, once every seven days. In some embodiments, the method comprises orally administering about 50 mg to about 450 mg of a crystalline form of the compound of Formula Ib (e.g., crystalline Form I) to the patient, once every seven days. In some embodiments, the method comprises orally administering about 75 mg to about 425 mg of a crystalline form of the compound of Formula Ib (e.g., crystalline Form I) to the patient, once every seven days.

In some embodiments, the method comprises orally administering about 75 mg to about 125 mg of a crystalline form of the compound of Formula Ib (e.g., crystalline Form I) to the patient, once every seven days. In some embodiments, the method comprises orally administering about 100 mg of a crystalline form of the compound of Formula Ib (e.g., crystalline Form I) to the patient, once every seven days.

In some embodiments, the method comprises orally administering about 175 mg to about 225 mg of a crystalline form of the compound of Formula Ib (e.g., crystalline Form I) to the patient, once every seven days. In some embodiments, the method comprises orally administering about 200 mg of a crystalline form of the compound of Formula Ib (e.g., crystalline Form I) to the patient, once every seven days.

In some embodiments, the method comprises orally administering about 275 mg to about 325 mg of a crystalline form of the compound of Formula Ib (e.g., crystalline Form I) to the patient, once every seven days. In some embodiments, the method comprises orally administering about 300 mg of a crystalline form of the compound of Formula Ib (e.g., crystalline Form I) to the patient, once every seven days.

In some embodiments, the method comprises orally administering about 375 mg to about 425 mg of a crystalline form of the compound of Formula Ib (e.g., crystalline Form I) to the patient, once every seven days. In some embodiments, the method comprises orally administering about 400 mg of a crystalline form of the compound of Formula Ib (e.g., crystalline Form I) to the patient, once every seven days.

In some embodiments, the tablet comprises about 50 mg to about 300 mg of mannitol, for example, about 50 mg, about 100 mg, about 150 mg, about 200 mg, about 250 mg, about 300 mg or about 300 mg of mannitol.

In some embodiments, the tablet comprises about 50 mg to about 100 mg of mannitol. In some embodiments, the tablet comprises about 55 mg to about 75 mg of mannitol. In some embodiments, the tablet comprises about 65 mg to about 75 mg of mannitol. In some embodiments, the tablet comprises about 67 mg to about 68 mg of mannitol. In some embodiments, the tablet comprises about 67.5 mg of mannitol.

In some embodiments, the tablet comprises about 100 mg to about 150 mg of mannitol. In some embodiments, the tablet comprises about 120 mg to about 140 mg of mannitol. In some embodiments, the tablet comprises about 130 mg to about 140 mg of mannitol. In some embodiments, the tablet comprises about 134 mg to about 136 mg of mannitol. In some embodiments, the tablet comprises about 135 mg of mannitol.

In some embodiments, the tablet comprises about 150 mg to about 250 mg of mannitol. In some embodiments, the tablet comprises about 190 mg to about 210 mg of mannitol. In some embodiments, the tablet comprises about 200 mg to about 205 mg of mannitol. In some embodiments, the tablet comprises about 202 mg to about 203 mg of mannitol. In some embodiments, the tablet comprises about 202.5 mg of mannitol.

In some embodiments, the tablet comprises about 250 mg to about 300 mg of mannitol. In some embodiments, the tablet comprises about 260 mg to about 280 mg of mannitol. In some embodiments, the tablet comprises about 265 mg to about 275 mg of mannitol. In some embodiments, the tablet comprises about 269 mg to about 271 mg of mannitol. In some embodiments, the tablet comprises about 270 mg of mannitol.

In some embodiments, the tablet comprises about 50 mg to about 300 mg of microcrystalline cellulose, for example, about 50 mg, about 100 mg, about 150 mg, about 200 mg, about 250 mg, about 300 mg or about 300 mg of microcrystalline cellulose.

In some embodiments, the tablet comprises about 50 mg to about 100 mg of microcrystalline cellulose. In some embodiments, the tablet comprises about 55 mg to about 75 mg of microcrystalline cellulose. In some embodiments, the tablet comprises about 65 mg to about 75 mg of microcrystalline cellulose. In some embodiments, the tablet comprises about 68 mg to about 69 mg of microcrystalline cellulose. In some embodiments, the tablet comprises about 68.75 mg of microcrystalline cellulose.

In some embodiments, the tablet comprises about 100 mg to about 150 mg of microcrystalline cellulose. In some embodiments, the tablet comprises about 120 mg to about 140 mg of microcrystalline cellulose. In some embodiments, the tablet comprises about 135 mg to about 140 mg of microcrystalline cellulose. In some embodiments, the tablet comprises about 136 mg to about 138 mg of microcrystalline cellulose. In some embodiments, the tablet comprises about 137.5 mg of microcrystalline cellulose.

In some embodiments, the tablet comprises about 150 mg to about 250 mg of microcrystalline cellulose. In some embodiments, the tablet comprises about 195 mg to about 215 mg of microcrystalline cellulose. In some embodiments, the tablet comprises about 205 mg to about 210 mg of microcrystalline cellulose. In some embodiments, the tablet comprises about 206 mg to about 207 mg of microcrystalline cellulose. In some embodiments, the tablet comprises about 206.25 mg of microcrystalline cellulose.

In some embodiments, the tablet comprises about 250 mg to about 300 mg of microcrystalline cellulose. In some embodiments, the tablet comprises about 265 mg to about 285 mg of microcrystalline cellulose. In some embodiments, the tablet comprises about 270 mg to about 280 mg of microcrystalline cellulose. In some embodiments, the tablet comprises about 274 mg to about 276 mg of microcrystalline cellulose. In some embodiments, the tablet comprises about 275 mg of microcrystalline cellulose.

In some embodiments, the tablet comprises about 5 mg to about 50 mg of crospovidone, for example, about 5 mg, about 10 mg, about 15 mg, about 20 mg, about 25 mg, about 30 mg, about 35 mg, about 40 mg, about 45 mg, or about 50 mg of crospovidone.

In some embodiments, the tablet comprises about 5 mg to about 15 mg of crospovidone. In some embodiments, the tablet comprises about 7 mg to about 13 mg of crospovidone. In some embodiments, the tablet comprises about 9 mg to about 11 mg of crospovidone. In some embodiments, the tablet comprises about 10 mg of crospovidone.

In some embodiments, the tablet comprises about 15 mg to about 25 mg of crospovidone. In some embodiments, the tablet comprises about 17 mg to about 23 mg of crospovidone. In some embodiments, the tablet comprises about 19 mg to about 21 mg of crospovidone. In some embodiments, the tablet comprises about 20 mg of crospovidone. In some embodiments, the tablet comprises about 25 mg to about 35 mg of crospovidone. In some embodiments, the tablet comprises about 27 mg to about 33 mg of crospovidone. In some embodiments, the tablet comprises about 29 mg to about 31 mg of crospovidone. In some embodiments, the tablet comprises about 30 mg of crospovidone. In some embodiments, the tablet comprises about 35 mg to about 45 mg of crospovidone. In some embodiments, the tablet comprises about 37 mg to about 43 mg of crospovidone. In some embodiments, the tablet comprises about 39 mg to about 41 mg of crospovidone. In some embodiments, the tablet comprises about 40 mg of crospovidone.

In some embodiments, the tablet comprises about 1 mg to about 20 mg of magnesium stearate, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg magnesium stearate.

In some embodiments, the tablet comprises about 1 mg to about 6 mg of magnesium stearate. In some embodiments, the tablet comprises about 2 mg to about 5 mg of magnesium stearate. In some embodiments, the tablet comprises about 3 mg to about 4 mg of magnesium stearate. In some embodiments, the tablet comprises about 3.75 mg of magnesium stearate.

In some embodiments, the tablet comprises about 5 mg to about 10 mg of magnesium stearate. In some embodiments, the tablet comprises about 6 mg to about 9 mg of magnesium stearate. In some embodiments, the tablet comprises about 6 mg to about 8 mg of magnesium stearate. In some embodiments, the tablet comprises about 7.5 mg of magnesium stearate.

In some embodiments, the tablet comprises about 9 mg to about 14 mg of magnesium stearate. In some embodiments, the tablet comprises about 10 mg to about 13 mg of magnesium stearate. In some embodiments, the tablet comprises about 11 mg to about 12 mg of magnesium stearate. In some embodiments, the tablet comprises about 11.25 mg of magnesium stearate.

In some embodiments, the tablet comprises about 10 mg to about 20 mg of magnesium stearate. In some embodiments, the tablet comprises about 13 mg to about 17 mg of magnesium stearate. In some embodiments, the tablet comprises about 14 mg to about 16 mg of magnesium stearate. In some embodiments, the tablet comprises about 15 mg of magnesium stearate.

In some embodiments, the tablet provided herein comprises or consists essentially of:
about 5 mg to about 500 mg of the compound of Formula Ia or Ib, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises or consists essentially of:
about 75 mg to about 125 mg of the compound of Formula Ia or Ib, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises or consists essentially of:

about 100 mg of the compound of Formula Ia or Ib, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the tablet provided herein comprises or consists essentially of:
about 75 mg to about 125 mg of the compound of Formula Ia, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises or consists essentially of:
about 100 mg of the compound of Formula Ia, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the tablet provided herein comprises or consists essentially of:
about 75 mg to about 125 mg of the compound of Formula Ib, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises or consists essentially of:
about 100 mg of the compound of Formula Ib, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the tablet provided herein comprises or consists essentially of:
about 75 mg to about 125 mg of a crystalline form of the compound of Formula Ib;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises or consists essentially of:
about 100 mg of a crystalline form of the compound of Formula Ib;
about 67.5 mg of mannitol;
about 68.75 mg of microcrystalline cellulose;
about 10 mg of crospovidone; and
about 3.75 mg of magnesium stearate.

In some embodiments, the tablet provided herein comprises or consists essentially of:
about 75 mg to about 125 mg of the compound of Formula Ib, crystalline Form I;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises or consists essentially of:
about 100 mg of the compound of Formula Ib, crystalline Form I;
about 67.5 mg of mannitol;
about 68.75 mg of microcrystalline cellulose;
about 10 mg of crospovidone; and
about 3.75 mg of magnesium stearate.

In some embodiments, the tablet provided herein comprises or consists essentially of:
about 175 mg to about 225 mg of the compound of Formula Ia or Ib, or a pharmaceutically acceptable salt thereof;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises or consists essentially of:
about 200 mg of the compound of Formula Ia or Ib, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the tablet provided herein comprises or consists essentially of:
about 5 mg to about 500 mg of the compound of Formula Ia, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises or consists essentially of:
about 175 mg to about 225 mg of the compound of Formula Ia, or a pharmaceutically acceptable salt thereof;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises or consists essentially of:
about 200 mg of the compound of Formula Ia, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the tablet provided herein comprises or consists essentially of:
about 5 mg to about 500 mg of the compound of Formula Ia;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises or consists essentially of:
about 175 mg to about 225 mg of the compound of Formula Ia;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises or consists essentially of:
about 200 mg of the compound of Formula Ia;
about 135 mg of mannitol;
about 137.5 mg of microcrystalline cellulose;
about 20 mg of crospovidone; and
about 7.5 mg of magnesium stearate.

In some embodiments, the tablet provided herein comprises or consists essentially of:
about 5 mg to about 500 mg of the compound of Formula Ib, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises or consists essentially of:
about 175 mg to about 225 mg of the compound of Formula Ib, or a pharmaceutically acceptable salt thereof;

about 100 mg to about 150 mg of mannitol;

about 100 mg to about 150 mg of microcrystalline cellulose;

about 15 mg to about 25 mg of crospovidone; and

about 5 mg to about 10 mg of magnesium stearate.

In some embodiments, the tablet provided herein comprises or consists essentially of:
about 200 mg of the compound of the compound of Formula Ib, or a pharmaceutically acceptable salt thereof;

about 135 mg of mannitol;

about 137.5 mg of microcrystalline cellulose;

about 20 mg of crospovidone; and

about 7.5 mg of magnesium stearate.

In some embodiments, the tablet provided herein comprises or consists essentially of:
about 5 mg to about 500 mg of the compound of Formula Ib;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises or consists essentially of:
about 175 mg to about 225 mg of the compound of Formula Ib;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises or consists essentially of:
about 200 mg of the compound of the compound of Formula Ib;
about 135 mg of mannitol;
about 137.5 mg of microcrystalline cellulose;
about 20 mg of crospovidone; and
about 7.5 mg of magnesium stearate.

In some embodiments, the tablet provided herein comprises or consists essentially of:
about 5 mg to about 500 mg of a crystalline form of the compound of Formula Ib;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises or consists essentially of:
about 175 mg to about 225 mg of a crystalline form of the compound of Formula Ib;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises or consists essentially of:
about 200 mg of a crystalline form of the compound of Formula Ib;
about 135 mg of mannitol;
about 137.5 mg of microcrystalline cellulose;
about 20 mg of crospovidone; and
about 7.5 mg of magnesium stearate.

In some embodiments, the tablet provided herein comprises or consists essentially of:

about 5 mg to about 500 mg of a compound of Formula Ib, crystalline Form I;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises or consists essentially of:
about 175 mg to about 225 mg of a compound of Formula Ib, crystalline Form I;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises or consists essentially of:
about 200 mg of a compound of Formula Ib, crystalline Form I;
about 135 mg of mannitol;
about 137.5 mg of microcrystalline cellulose;
about 20 mg of crospovidone; and
about 7.5 mg of magnesium stearate.

In some embodiments, the tablet provided herein comprises or consists essentially of:
about 275 mg to about 325 mg of the compound of Formula Ia or Ib, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises or consists essentially of:
about 300 mg of the compound of Formula Ia or Ib, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the tablet provided herein comprises or consists essentially of:
about 275 mg to about 325 mg of the compound of Formula Ia, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises or consists essentially of:
about 300 mg of the compound of Formula Ia, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the tablet provided herein comprises or consists essentially of:
about 275 mg to about 325 mg of the compound of Formula Ib, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises or consists essentially of:
about 300 mg of the compound of Formula Ib, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the tablet provided herein comprises or consists essentially of:
about 275 mg to about 325 mg of a crystalline form of the compound of Formula Ib;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises or consists essentially of:
about 300 mg of a crystalline form of the compound of Formula Ib;
about 202.5 mg of mannitol;
about 206.25 mg of microcrystalline cellulose;
about 30 mg of crospovidone; and
about 11.25 mg of magnesium stearate.

In some embodiments, the tablet provided herein comprises or consists essentially of:
about 275 mg to about 325 mg of a compound of Formula Ib, crystalline Form I;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises or consists essentially of:
about 300 mg of a compound of Formula Ib, crystalline Form I;
about 202.5 mg of mannitol;
about 206.25 mg of microcrystalline cellulose;
about 30 mg of crospovidone; and
about 11.25 mg of magnesium stearate.

In some embodiments, the tablet provided herein comprises or consists essentially of:
about 375 mg to about 425 mg of the compound of Formula Ia or Ib, or a pharmaceutically acceptable salt thereof;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises or consists essentially of:
about 400 mg of the compound of Formula Ia or Ib, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the tablet provided herein comprises or consists essentially of:
about 375 mg to about 425 mg of the compound of Formula Ia, or a pharmaceutically acceptable salt thereof;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises or consists essentially of:
about 400 mg of the compound of Formula Ia, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the tablet provided herein comprises or consists essentially of:
about 375 mg to about 425 mg of the compound of Formula Ib, or a pharmaceutically acceptable salt thereof;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises or consists essentially of:
about 400 mg of the compound of Formula Ib, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the tablet provided herein comprises or consists essentially of:
about 375 mg to about 425 mg of a crystalline form of a compound of Formula Ib;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises or consists essentially of:
about 400 mg of a crystalline form of a compound of Formula Ib;
about 270 mg of mannitol;
about 275 mg of microcrystalline cellulose;
about 40 mg of crospovidone; and
about 15 mg of magnesium stearate.

In some embodiments, the tablet provided herein comprises or consists essentially of:
about 375 mg to about 425 mg of a compound of Formula Ib, crystalline Form I;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises or consists essentially of:
about 400 mg of a compound of Formula Ib, crystalline Form I;
about 270 mg of mannitol;
about 275 mg of microcrystalline cellulose;
about 40 mg of crospovidone; and
about 15 mg of magnesium stearate.

The tablets disclosed herein may be uncoated or coated (in which case they include an outer film coat). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water-soluble material included in the film coating of the tablets may include a single polymer material, it may also be formed using a mixture of more than one polymer. The coating may be white or colored. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional colouring such as iron oxide or indigo carmine or iron oxide yellow or FD&C yellow #6). The amount of coating will generally be between about 1-8% of the uncoated tablet's weight.

In some embodiments of the methods provided herein, each tablet provided herein further comprises an outer film coat. In some embodiments of the methods provided herein, each tablet comprising a compound of Formula Ia, or a pharmaceutically acceptable salt thereof, provided herein further comprises an outer film coat. In some embodiments of the methods provided herein, each tablet comprising a compound of Formula Ib, or a pharmaceutically acceptable salt thereof, provided herein further comprises an outer film coat.

In some embodiments of the methods provided herein, the outer film coat provides from about 1% to about 10% weight gain based on the uncoated tablet. In some embodiments, the amount of coating is 1% of the uncoated tablet's weight. In some embodiments, the amount of coating is 2% of the uncoated tablet's weight. In some embodiments, the amount of coating is 3% of the uncoated tablet's weight. In some embodiments, the amount of coating is 4% of the uncoated tablet's weight. In some embodiments, the amount of coating is 5% of the uncoated tablet's weight. In some embodiments, the amount of coating is 6% of the uncoated tablet's weight. In some embodiments, the amount of coating is 7% of the uncoated tablet's weight. In some embodiments, the amount of coating is 8% of the uncoated tablet's weight. In some embodiments, the amount of coating is 9% of the uncoated tablet's weight. In some embodiments, the amount of coating is 10% of the uncoated tablet's weight.

In some embodiments, the coating is Opadry II Purple 85F140073. Opadry II Purple 85F140073 contains 40.00% w/w polyvinyl alcohol (USP/Ph. Eur.), 24.20% w/w titanium dioxide (USP/Ph. Eur.), 20.20% w/w macrogol/polyethylene glycol (USP/Ph. Eur.), 14.80% w/w talc (USP/Ph. Eur.), 0.58% w/w iron oxide red (NF), and 0.22% w/w ferrosoferric oxide/black iron oxide (NF).

In some embodiments, the coating is Opadry II Gray 85F97517. Opadry II Gray 85F97517 contains 40.00% w/w polyvinyl alcohol (USP/Ph.Eur.), 24.74% w/w titanium dioxide, (USP/Ph.Eur.), 20.20% w/w macrogol/polyethylene glycol (NF/Ph.Eur.), 14.80% (w/w) talc (USP/Ph.Eur.), and 0.26% w/w ferrosoferric oxide/black iron oxide (NF). In some embodiments, the coating is Opadry TF Gray 273F17005. Opadry TF Gray 273F175005 contains 37.000% w/w polyvinyl alcohol (USP/FCC/PhEur/JPE/ChP/GB), 34.850% w/w calcium carbonate (USP/FCC/PhEur/JP/JECFA/JSFA/GB), 14.500% w/w microcrystalline cellulose (NF/PhEur/JP/ChP), 7.000% w/w macrogol 6000 JP/PEG (USP/FCC/PhEur/JECFA/JP), 5.000% w/w magnesium aluminometasilicate (Type IA NF/Type A PhEur/JP), 1.000% carnauba wax (NF/FCC/PhEur/JP/ChP/GB), 0.500% xanthan gum (NF/FCC/PhEur/JPE/ChP/JECFA), 0.150% w/w ferrosoferric oxide (NF)/black iron oxide (JPE/JECFA/ChP).

### Pharmaceutical Compositions of Compounds of Formula II (Encequidar)

In some embodiments, the pharmaceutical composition (i.e. formulation) comprising the compound of Formula II, or a pharmaceutically acceptable salt thereof, is suitable for oral administration. In some embodiments, the pharmaceutical composition comprising the compound of Formula II, or a pharmaceutically acceptable salt thereof, is a suspension or solution.

In some embodiments, the pharmaceutical composition comprising the compound of Formula II, or a pharmaceutically acceptable salt thereof, is a solution. In some embodiments, the pharmaceutical composition comprising the compound of Formula II, or a pharmaceutically acceptable salt thereof, is an injectable solution. In some embodiments, the pharmaceutical composition comprising the compound of Formula II, or a pharmaceutically acceptable salt thereof, is a solution suitable for oral administration. In some embodiments, the pharmaceutical composition comprising the compound of Formula II, or a pharmaceutically acceptable salt thereof, is an injectable solution suitable for oral administration.

In some embodiments, the pharmaceutical composition comprising the compound of Formula II, or a pharmaceutically acceptable salt thereof, is a suspension. In some embodiments, the pharmaceutical composition comprising the compound of Formula II, or a pharmaceutically acceptable salt thereof, is an injectable suspension. In some embodiments, the pharmaceutical composition comprising the compound of Formula II, or a pharmaceutically acceptable salt thereof, is a suspension suitable for oral administration. In some embodiments, the pharmaceutical composition comprising the compound of Formula II, or a pharmaceutically acceptable salt thereof, is an injectable suspension suitable for oral administration.

In some embodiments, the suspension comprising the compound of Formula II, or a pharmaceutically acceptable salt thereof, comprises about 1 mg/mL to about 10 mg/mL of the compound of Formula II, or a pharmaceutically acceptable salt thereof, for example, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL of the compound of Formula II, or a pharmaceutically acceptable salt thereof.

In some embodiments, the suspension comprising the compound of Formula II, or a pharmaceutically acceptable salt thereof, comprises about 1 mg/mL to about 5 mg/mL of the compound of Formula II, or a pharmaceutically acceptable salt thereof.

In some embodiments, the suspension comprising the compound of Formula II, or a pharmaceutically acceptable salt thereof, comprises about 1 mg/mL to about 3 mg/mL of the compound of Formula II, or a pharmaceutically acceptable salt thereof.

In some embodiments, the suspension comprising the compound of Formula II, or a pharmaceutically acceptable salt thereof, comprises about 1.5 mg/mL to about 2.5 mg/mL of the compound of Formula II, or a pharmaceutically acceptable salt thereof.

In some embodiments, the suspension comprising the compound of Formula II, or a pharmaceutically acceptable salt thereof, comprises about 1.9 mg/mL to about 2.1 mg/mL of the compound of Formula II, or a pharmaceutically acceptable salt thereof.

In some embodiments, the suspension comprising the compound of Formula II, or a pharmaceutically acceptable salt thereof, comprises about 2 mg/mL of the compound of Formula II, or a pharmaceutically acceptable salt thereof.

In some embodiments, the suspension comprising the compound of Formula II, or a pharmaceutically acceptable salt thereof, comprises carboxymethyl cellulose.

In some embodiments, the suspension comprising the compound of Formula II, or a pharmaceutically acceptable salt thereof, comprises about 0.1 w/w % to about 2 w/w % carboxymethyl cellulose, for example, about 0.1 w/w %, about 0.2 w/w %, about 0.3 w/w %, about 0.4 w/w %, about 0.5 w/w %, about 0.6 w/w %, about 0.7 w/w %, about 0.8 w/w %, about 0.9 w/w %, about 1 w/w %, about 1.1 w/w %, about 1.2 w/w %, about 1.3 w/w %, about 1.4 w/w %, about 1.5 w/w %, about 1.6 w/w %, about 1.7 w/w %, about 1.8 w/w %, about 1.9 w/w %, or about 2 w/w % carboxymethyl cellulose.

In some embodiments, the suspension comprising the compound of Formula II, or a pharmaceutically acceptable salt thereof, comprises about 0.1 w/w % to about 1 w/w % carboxymethyl cellulose.

In some embodiments, the suspension comprising the compound of Formula II, or a pharmaceutically acceptable salt thereof, comprises about 0.4 w/w % to about 0.6 w/w % carboxymethyl cellulose.

In some embodiments, the suspension comprising the compound of Formula II, or a pharmaceutically acceptable salt thereof, comprises about 0.5 w/w % carboxymethyl cellulose. In some embodiments, the suspension comprising the compound of Formula II, or a pharmaceutically acceptable salt thereof, comprises about 98 w/w % to about 99.9 w/w % water, for example, about 98 w/w %, about 98.1 w/w %, about 98.2 w/w %, about 98.3 w/w %, about 98.4 w/w %, about 98.5 w/w %, about 98.6 w/w %, about 98.7 w/w %, about 98.8 w/w %, about 98.9 w/w %, about 99 w/w %, about 99.2 w/w %, about 99.3 w/w %, about 99.4 w/w %, about 99.5 w/w %, about 99.6 w/w %, about 99.7 w/w %, about 99.8 w/w %, or about 99.9 w/w % water.

In some embodiments, the suspension comprising the compound of Formula II, or a pharmaceutically acceptable salt thereof, comprises about 99 w/w % to about 99.9 w/w % water. In some embodiments, the suspension comprising the compound of Formula II, or a pharmaceutically acceptable salt thereof, comprises about 99.5 w/w % water.

In some embodiments, the suspension comprising the compound of Formula II, or a pharmaceutically acceptable salt thereof, comprises carboxymethyl cellulose and water.

In some embodiments, the suspension consists essentially of the compound of Formula II, or a pharmaceutically acceptable salt thereof, carboxymethyl cellulose and water.

In some embodiments, the suspension comprising the compound of Formula II, or a pharmaceutically acceptable salt thereof, comprises about 1 mg/mL to about 10 mg/mL of the compound of Formula II, or a pharmaceutically acceptable salt thereof, about 0.1 w/w % to about 2 w/w % carboxymethyl cellulose, and about 98 w/w % to about 99.9 w/w % water.

In some embodiments, the suspension consists essentially of about 1 mg/mL to about 10 mg/mL of the compound of Formula II, or a pharmaceutically acceptable salt thereof, about 0.1 w/w % to about 2 w/w % carboxymethyl cellulose, and about 98 w/w % to about 99.9 w/w % water.

In some embodiments, the suspension comprising the compound of Formula II, or a pharmaceutically acceptable salt thereof, comprises about 1 mg/mL to about 5 mg/ml of the compound of Formula II, or a pharmaceutically acceptable salt thereof, about 0.1 w/w % to about 1 w/w % carboxymethyl cellulose, and about 99 w/w % to about 99.9 w/w % water.

In some embodiments, the suspension consists essentially of about 1 mg/mL to about 5 mg/mL of the compound of Formula II, or a pharmaceutically acceptable salt thereof, about 0.1 w/w % to about 1 w/w % carboxymethyl cellulose, and about 99 w/w % to about 99.9 w/w % water.

In some embodiments, the solution comprising the compound of Formula II, or a pharmaceutically acceptable salt thereof, comprises about 2 mg/mL of the compound of Formula II, or a pharmaceutically acceptable salt thereof, about 0.5 w/w % carboxymethyl cellulose, and about 99.5 w/w % water.

In some embodiments, the solution comprising the compound of Formula II, or a pharmaceutically acceptable salt thereof, comprises Kolliphor HS-15, N-methylpyrrolidone, PEG 300, water, or any combination thereof.

In some embodiments, the solution comprising the compound of Formula II, or a pharmaceutically acceptable salt thereof, comprises Kolliphor HS-15.

In some embodiments, the solution comprising the compound of Formula II, or a pharmaceutically acceptable salt thereof, comprises about 5 w/w % to about 15 w/w % Kolliphor HS-15, for example, about 6 w/w %, about 7 w/w %, about 8 w/w %, about 9 w/w %, about 10 w/w %, about 11 w/w %, about 12 w/w %, about 13 w/w %, about 14 w/w %, or about 15 w/w % Kolliphor HS-15.

In some embodiments, the solution comprising the compound of Formula II, or a pharmaceutically acceptable salt thereof, comprises about 8 w/w % to about 12 w/w % Kolliphor HS-15.

In some embodiments, the solution comprising the compound of Formula II, or a pharmaceutically acceptable salt thereof, comprises about 9 w/w % to about 11 w/w % Kolliphor HS-15.

In some embodiments, the solution comprising the compound of Formula II, or a pharmaceutically acceptable salt thereof, comprises about 10 w/w % Kolliphor HS-15.

In some embodiments, the solution comprising the compound of Formula II, or a pharmaceutically acceptable salt thereof, comprises N-methylpyrrolidone.

In some embodiments, the solution comprising the compound of Formula II, or a pharmaceutically acceptable salt thereof, comprises about 1 w/w % to about 10 w/w % N-methylpyrrolidone, for example, about 1 w/w %, about 2 w/w %, about 3 w/w %, about 4 w/w %, about 5 w/w %, about 6 w/w %, about 7 w/w %, about 8 w/w %, about 9 w/w %, about 10 w/w % N-methylpyrrolidone.

In some embodiments, the solution comprising the compound of Formula II, or a pharmaceutically acceptable salt thereof, comprises about 3 w/w % to about 7 w/w % N-methylpyrrolidone.

In some embodiments, the solution comprising the compound of Formula II, or a pharmaceutically acceptable salt thereof, comprises about 4 w/w % to about 6 w/w % N-methylpyrrolidone.

In some embodiments, the solution comprising the compound of Formula II, or a pharmaceutically acceptable salt thereof, comprises about 5 w/w % N-methylpyrrolidone.

In some embodiments, the solution comprising the compound of Formula II, or a pharmaceutically acceptable salt thereof, comprises polyethylene glycol 300 (i.e., PEG 300).

In some embodiments, the solution comprising the compound of Formula II, or a pharmaceutically acceptable salt thereof, comprises about 20 w/w % to about 40 w/w % PEG 300, for example, about 20 w/w %, about 21 w/w %, about 22 w/w %, about 23 w/w %, about 24 w/w %, about 25 w/w %, about 26 w/w %, about 27 w/w %, about 28 w/w %, about 29 w/w %, about 30 w/w %, about 31 w/w %, about 32 w/w %, about 33 w/w %, about 34 w/w %, about 35 w/w %, about 36 w/w %, about 37 w/w %, about 38 w/w %, about 39 w/w %, or about 40 w/w % PEG 300.

In some embodiments, the solution comprising the compound of Formula II, or a pharmaceutically acceptable salt thereof, comprises about 25 w/w % to about 35 w/w % PEG 300.

In some embodiments, the solution comprising the compound of Formula II, or a pharmaceutically acceptable salt thereof, comprises about 29 w/w % to about 31 w/w % PEG 300.

In some embodiments, the solution comprising the compound of Formula II, or a pharmaceutically acceptable salt thereof, comprises about 30 w/w % PEG 300.

In some embodiments, the solution comprising the compound of Formula II, or a pharmaceutically acceptable salt thereof, comprises water.

In some embodiments, the solution comprising the compound of Formula II, or a pharmaceutically acceptable salt thereof, comprises about 45 w/w % to about 65 w/w % water, for example, about 45 w/w %, about 46 w/w %, about 47 w/w %, about 48 w/w %, about 49 w/w %, about 50 w/w %, about 51 w/w %, about 52 w/w %, about 53 w/w %, about 54 w/w %, about 55 w/w %, about 56 w/w %, about 57 w/w %, about 58 w/w %, about 59 w/w %, or about 60 w/w % water.

In some embodiments, the solution comprising the compound of Formula II, or a pharmaceutically acceptable salt thereof, comprises about 50 w/w % to about 60 w/w % water.

In some embodiments, the solution comprising the compound of Formula II, or a pharmaceutically acceptable salt thereof, comprises about 54 w/w % to about 56 w/w % water.

In some embodiments, the solution comprising the compound of Formula II, or a pharmaceutically acceptable salt thereof, comprises about 55 w/w % water.

In some embodiments, the solution comprising the compound of Formula II, or a pharmaceutically acceptable salt thereof, comprises Kolliphor HS-15, N-methylpyrrolidone, PEG 300, and water.

In some embodiments, the solution consists essentially of the compound of Formula II, or a pharmaceutically acceptable salt thereof, Kolliphor HS-15, N-methylpyrrolidone, PEG 300, and water.

In some embodiments, the solution comprising the compound of Formula II, or a pharmaceutically acceptable salt thereof, comprises about 1 mg/mL to about 10 mg/mL of the compound of Formula II, or a pharmaceutically acceptable salt thereof, about 5 w/w % to about 15 w/w % Kolliphor HS-15, about 1 w/w % to about 10 w/w % N-methylpyrrolidone, about 20 w/w % to about 40 w/w % PEG 300, and about 45 w/w % to about 65 w/w % water.

In some embodiments, the solution consists essentially of about 1 mg/mL to about 10 mg/mL of the compound of Formula II, or a pharmaceutically acceptable salt thereof, about 5 w/w % to about 15 w/w % Kolliphor HS-15, about 1 w/w % to about 10 w/w % N-methylpyrrolidone, about 20 w/w % to about 40 w/w % PEG 300, and about 45 w/w % to about 65 w/w % water.

In some embodiments, the solution comprising the compound of Formula II, or a pharmaceutically acceptable salt thereof, comprises about 1 mg/mL to about 5 mg/mL of the compound of Formula II, or a pharmaceutically acceptable salt thereof, about 8 w/w % to about 12 w/w % Kolliphor HS-15, about 3 w/w % to about 7 w/w % N-methylpyrrolidone, about 25 w/w % to about 35 w/w % PEG 300, and about 50 w/w % to about 60 w/w % water.

In some embodiments, the solution consists essentially of about 1 mg/mL to about 5 mg/mL of the compound of Formula II, or a pharmaceutically acceptable salt thereof, about 8 w/w % to about 12 w/w % Kolliphor HS-15, about 3 w/w % to about 7 w/w % N-methylpyrrolidone, about 25 w/w % to about 35 w/w % PEG 300, and about 50 w/w % to about 60 w/w % water.

In some embodiments, the solution comprising the compound of Formula II, or a pharmaceutically acceptable salt thereof, comprises about 1.5 mg/mL to about 2.5 mg/mL of the compound of Formula II, or a pharmaceutically acceptable salt thereof, about 9 w/w % to about 11 w/w % Kolliphor HS-15, about 4 w/w % to about 6 w/w % N-methylpyrrolidone, about 29 w/w % to about 31 w/w % PEG 300, and about 54 w/w % to about 56 w/w % water.

In some embodiments, the solution consists essentially of about 1.5 mg/mL to about 2.5 mg/mL of the compound of Formula II, or a pharmaceutically acceptable salt thereof, about 9 w/w % to about 11 w/w % Kolliphor HS-15, about 4 w/w % to about 6 w/w % N-methylpyrrolidone, about 29 w/w % to about 31 w/w % PEG 300, and about 54 w/w % to about 56 w/w % water.

In some embodiments, the solution comprising the compound of Formula II, or a pharmaceutically acceptable salt thereof, comprises about 2.0 mg/mL of the compound of Formula II, or a pharmaceutically acceptable salt thereof, about 10 w/w % Kolliphor HS-15, about 5 w/w % N-methylpyrrolidone, about 30 w/w % PEG 300, and about 55 w/w % water.

In some embodiments, the solution or suspension comprising the compound of Formula II, or a pharmaceutically acceptable salt thereof, is administered to the patient once every seven days.

In some embodiments, the solution or suspension comprising the compound of Formula II, or a pharmaceutically acceptable salt thereof, is orally administered to the patient once every seven days.

In some embodiments, the solution or suspension comprising the compound of Formula II, or a pharmaceutically acceptable salt thereof, is administered to the patient once every month.

In some embodiments, the solution or suspension comprising the compound of Formula II, or a pharmaceutically acceptable salt thereof, is orally administered to the patient once every month.

### Additional Combination Therapies

Patients being treated by administration of the compounds provided herein, or pharmaceutically acceptable salts thereof, often exhibit diseases or conditions that benefit from treatment with additional therapeutic agents, including agents that are therapeutic for Retroviridae infections, including an HIV infection. In some embodiments, the additional therapeutic agent is an agent that is therapeutic for an HIV infection. Thus, one aspect of the disclosure is a method of treating an HIV infection comprising administering the compounds, or a pharmaceutically acceptable salts thereof, or compositions of the present disclosure, in combination with one or more compounds useful for the treatment of an HIV infection to a subject, particularly a human subject, in need thereof.

In some embodiments, the methods provided herein further comprise administering to the patient one or more (i.e., one, two, three, four; one or two; one to three; or one to four) additional therapeutic agents, or a pharmaceutically acceptable salt thereof (i.e., administration of a compound of Formula I, or a pharmaceutically acceptable salt thereof, and a compound of

Formula II, or a pharmaceutically acceptable salt thereof, and one more additional therapeutic agents).

In some embodiments, the compounds, or a pharmaceutically acceptable salts thereof, or compositions of the present disclosure, are administered in combination with one, two, three, four or more additional therapeutic agents.

In some embodiments, the compounds, or a pharmaceutically acceptable salts thereof, or compositions of the present disclosure, are administered in combination with one additional therapeutic agent. In some embodiments, the compounds, or a pharmaceutically acceptable salts thereof, or compositions of the present disclosure, are administered in combination with two additional therapeutic agents. In some embodiments, the compounds, or a pharmaceutically acceptable salts thereof, or compositions of the present disclosure, are administered in combination with three additional therapeutic agents. In some embodiments, the compounds, or a pharmaceutically acceptable salts thereof, or compositions of the present disclosure, are administered in combination with four additional therapeutic agents. The one, two, three, four or more additional therapeutic agents can be different therapeutic agents selected from the same class of therapeutic agents, and/or they can be selected from different classes of therapeutic agents.

In some embodiments, when the compounds, or a pharmaceutically acceptable salts thereof, or compositions of the present disclosure, are administered in combination with one or more additional therapeutic agents as described herein, each compound or composition is administered as a simultaneous or sequential regimen. When administered sequentially, the combination may be administered in two or more administrations.

In some embodiments, the compounds, or a pharmaceutically acceptable salts thereof, or compositions of the present disclosure, are co-administered with one or more additional therapeutic agents.

Co-administration includes administration of unit dosages of the compounds, salts, and compositions provided herein, before or after administration of unit dosages of one or more additional therapeutic agents. The compounds, salts, and compositions provided herein may be administered within seconds, minutes, or hours of the administration of one or more additional therapeutic agents. For example, in some embodiments, a unit dose of a compound, salt, or composition provided herein, is administered first, followed within seconds or minutes by administration of a unit dose of one or more additional therapeutic agents. Alternatively, in some embodiments, a unit dose of one or more additional therapeutic agents is administered first, followed by administration of a unit dose of compound, salt, or composition provided herein, within seconds or minutes. In some embodiments, a unit dose of a compound, salt, or composition provided herein is administered first, followed, after a period of hours (i.e., 1-12 hours), by administration of a unit dose of one or more additional therapeutic agents. In other embodiments, a unit dose of one or more additional therapeutic agents is administered first, followed, after a period of hours (i.e., 1-12 hours), by administration of a unit dose of compound, salt, or composition provided herein.

### HIV Combination Therapy

In some embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents any are chosen from dolutegravir, cabotegravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911. Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), elsulfavirine (long acting injectable nanosuspension, HIV infection), and elsulfavirine (VM-1500). Additional non-limiting examples of non-nucleoside or non-nucleotide inhibitors of reverse transcriptase include the compounds disclosed in U.S. Pat. No. 10,548,898.

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

HIV Integrase Inhibitors Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017. Additional non-limiting examples of HIV integrase inhibitors include the compounds disclosed in U.S. Pat. No. 11,084,832.

Examples of HIV non-catalytic site, or allosteric, integrase inhibitors (NCINI) include, but are not limited to, CX-05045, CX-05168, and CX-14442.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

Examples of CD4 attachment inhibitors include, but are not limited to, ibalizumab and CADA analogs

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188,

N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQ1, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the compounds, salts, and compositions disclosed herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

Additional examples of HIV capsid inhibitors include, but are not limited to, those described in U.S. Patent Application Publication Nos. US2014221356 and US2016016973.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In some embodiments, the compounds, salts, and compositions disclosed herein are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18 (11): 671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAET1E; ULBP4); retinoic acid early transcript 1G (RAET1G; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In some embodiments, the compounds, salts, and compositions disclosed herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In some embodiments, the compounds, salts, and compositions disclosed herein are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18 (11): 671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In some embodiments, the compounds, salts, and compositions disclosed herein are combined with anti-TIGIT antibodies, such as BMS-986207, RG-6058, and AGEN-1307.

### TNF Receptor Superfamily (TNFRSF) Member Agonists or Activators

In some embodiments, the compounds, salts, and compositions disclosed herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106. Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific Natural Killer (NK)-Cell Engagers

In some embodiments, the compounds, salts, and compositions disclosed herein are combined with a bi-specific NK-cell engager (BiKE) or a tri-specific NK-cell engager (TrikE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TrikEs are described, e.g., in Felices et al., Methods Mol Biol. (2016) 1441:333-346; Fang et al., Semin Immunol. (2017) 31:37-54. Examples of trispecific NK cell engagers (TRIKE) include, but are not limited to, OXS-3550, HIV-TriKE, and CD16-IL-15-B7H3 Trike.

### Indoleamine-Pyrrole-2,3-Dioxygenase (IDO1) Inhibitors

In some embodiments, the compounds, salts, and compositions disclosed herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In some embodiments, the compounds, salts, and compositions disclosed herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the compounds, salts, and compositions disclosed herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the compounds, salts, and compositions disclosed herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the compounds, salts, and compositions disclosed herein are combined with a RIG-I modulator such as RGT-100, or NOD2 modulator, such as SB-9200, and IR-103.

### LAG-3 and TIM-3 Inhibitors

In some embodiments, the compounds, salts, and compositions disclosed herein are combined with an anti-TIM-3 antibody, such as TSR-022, LY-3321367, MBG-453, INCAGN-2390.

In some embodiments, the compounds, salts, and compositions disclosed herein are combined with an anti LAG-3 (Lymphocyte-activation) antibody, such as relatlimab (ONO-4482), LAG-525, MK-4280, REGN-3767, INCAGN2385.

### Interleukin Agonists

In some embodiments, the compounds, salts, and compositions disclosed herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated Il-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492 (7427): 118-22 (2012), Horwitz et al., Proc Natl Acad Sci USA, 110 (41): 16538-43 (2013), Scheid et al., Science, 333:1633-1637 (2011), Scheid et al., Nature, 458:636-640 (2009), Eroshkin et al, Nucleic Acids Res., 42 (Database issue): D1 133-9 (2014), Mascola et al., Immunol Rev., 254 (1): 225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as an additional therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173 (7): 1783-1795; Sajadi et al., J Infect Dis. (2016) 213 (1): 156-64; Klein et al., Nature, 492 (7427): 118-22 (2012), Horwitz et al., Proc Natl Acad Sci USA, 110 (41): 16538-43 (2013), Scheid et al., Science, 333:1633-1637 (2011), Scheid et al., Nature, 458:636-640 (2009), Eroshkin et al., Nucleic Acids Res., 42 (Database issue): D1 133-9 (2014), Mascola et al., Immunol Rev., 254 (1): 225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, DID2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7,VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e. rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9),

Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140 [delta] V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as Derma Vir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In some embodiments, the compounds, salts, and compositions disclosed herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In some embodiments, the compounds, salts, and compositions disclosed herein are combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ®(dolutegravir, abacavir, and lamivudine); dolutegravir, abacavir sulfate, and lamivudine; raltegravir; PEGylated raltegravir; raltegravir and lamivudine;

lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the compounds, salts, and compositions disclosed herein are combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In some embodiments, the compounds, salts, and compositions disclosed herein are combined with a first additional therapeutic agent chosen from dolutegravir, cabotegravir, darunavir, bictegravir, elsulfavirine, rilpivirine, and lenacapavir and a second additional therapeutic agent chosen from emtricitabine and lamivudine.

In some embodiments, the compounds, salts, and compositions disclosed herein are combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In some embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34−CXCR4+CCR5 ZFN T-cells, dual anti-CD4 CART-T cell therapy (CD4 CAR+C34−CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, doravirine, raltegravir, and long acting dolutegravir.

### EXAMPLES

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. In Vivo Pharmacokinetics of Lenacapavir Prodrug (Compound 2)+Encequidar in Male Beagle Dogs

A 55 mg fixed dose of Compound 2 was orally administered to each animal in a tablet formulation containing 4 w/w % crospovidone, 1.5 w/w % magnesium stearate, 26.97 w/w % mannitol, and 27.25 w/w % microcrystalline cellulose.

Dog PO suspension formulations of encequidar (2.0 mg/mL) in 0.5% carboxymethyl cellulose (CMC) and 99.5% water were prepared according to the following procedures. Non-uniform suspensions were sonicated for five minutes to break up any aggregates. Animals were dosed at 2.5 mL/kg for the 5 mg/kg dose within four hours of preparing the 0.5% CMC in 99.5% water suspension. A corresponding vehicle formulation was also prepared using 0.5% CMC and 99.5% water in the absence of encequidar. Study leg 1=administration of vehicle only; Study leg 2=administration of 2.0 mg/mL encequidar formulation.

Leg 1: All animals were administered a 2.5 mL/kg oral dose of vehicle 0.5% carboxymethyl cellulose (CMC) in water for injection (WFI) immediately followed by a 0.1 ml/kg intramuscular injection of pentagastrin approximately 30 minutes (+3 minutes) prior to administration of Compound 2 tablet.

Leg 2: All animals were administered a 5 mg/kg oral dose of encequidar in 0.5% carboxymethyl cellulose (CMC) in water for injection, immediately followed by a 0.1 mL/kg intramuscular injection of pentagastrin approximately 30 minutes (+3 minutes) prior to administration of Compound 2 tablet.

### Results

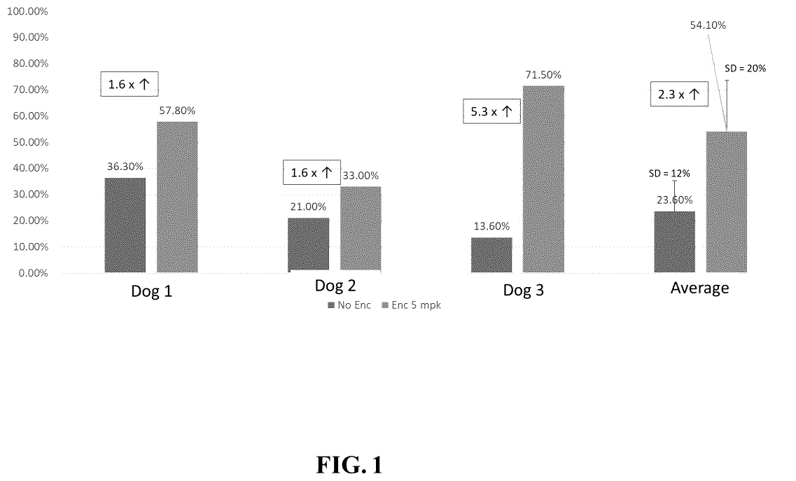

Table 2 shows PK parameters of lenacapavir after an oral dose at 55 mg fixed of Compound 2 in pentagastrin pretreated dogs, with and without encequidar (n=3). % F of lenacapavir was calculated using AUC_{inf} 18400 nM·h from average of Dog IV at 1 mg/kg. As shown in FIG. 1, administration of Compound 2, 55 mg fixed PO+encequidar solution showed an average 2.3× % F lenacapavir improvement.

TABLE 2

| | Animal ID | Dog 1 | Dog 2 | Dog 3 | Mean | SD |
|---|---|---|---|---|---|---|
| Leg 1 | AUCₗₐₛₜ(nM · h) | 23140 | 15408 | 10315 | 16287 | 6458 |
| (Compound 2 + | AUC_{inf} (nM · h) | 23456 | 15431 | 10358 | 16415 | 6604 |
| vehicle | Cₘₐₓ (nM) | 919 | 1200 | 735 | 951 | 234 |
| | Tₘₐₓ (h) | 4.00 | 4.00 | 4.00 | 4.00 | 0.00 |
| | lenacapavir % F | 36.3% | 21.0% | 13.6% | 23.6% | 11.6% |
| Leg 2 | AUCₗₐₛₜ(nM · h) | 38314 | 23552 | 53529 | 38465 | 14989 |
| (Compound 2 + | AUC_{inf} (nM · h) | 38741 | 23588 | 53571 | 38633 | 14992 |
| encequidar) | Cₘₐₓ (nM) | 1540 | 1080 | 2150 | 1590 | 537 |
| | Tₘₐₓ (h) | 2.00 | 4.00 | 2.00 | 2.7 | 1.2 |
| | lenacapavir % F | 57.8% | 33.0% | 71.5% | 54.1% | 19.5% |

Plasma concentration-time profiles for lenacapavir in individual animals are shown in Tables 3-4. BLQ: Below the limit of quantification <1.0 nM.

TABLE 3
Individual lenacapavir plasma concentration (nM)
in male beagle dogs following an oral dose of Compound
2 at 55 mg fixed dose with vehicle (Leg 1)

| Time (h) | Dog 1 | Dog 2 | Dog 3 | Mean | SD |
|---|---|---|---|---|---|
| 0.00 | BLQ | BLQ | BLQ | | |
| 0.25 | 23.4 | BLQ | 1.46 | 12.4 | |
| 0.50 | 1.22 | 32.5 | 6.21 | 13.3 | 16.8 |
| 1.00 | 346 | 14.7 | 159 | 173 | 166 |
| 2.00 | 886 | 1120 | 719 | 908 | 201 |
| 4.00 | 919 | 1200 | 735 | 951 | 234 |
| 6.00 | 818 | 832 | 468 | 706 | 206 |
| 8.00 | 636 | 652 | 369 | 552 | 159 |
| 12.0 | 464 | 466 | 242 | 391 | 129 |
| 24.0 | 287 | 163 | 160 | 203 | 72.5 |
| 48.0 | 147 | 28.8 | 25.7 | 67.2 | 69.2 |
| 72.0 | 79.9 | 10.4 | 5.75 | 32.0 | 41.5 |
| 96.0 | 37.5 | 2.86 | 2.53 | 14.3 | 20.1 |
| 120 | 24.8 | 1.09 | BLQ | 12.9 | |
| 144 | 10.6 | BLQ | BLQ | 10.6 | |
| 168 | 7.47 | BLQ | BLQ | 7.47 | |

TABLE 4
Individual lenacapavir plasma concentration (nM) in
male beagle dogs following an oral dose of Compound
2 at 55 mg fixed dose with encequidar (Leg 2)

| Time (h) | Dog 1 | Dog 2 | Dog 3 | Mean | SD |
|---|---|---|---|---|---|
| 0.00 | BLQ | BLQ | BLQ | | |
| 0.25 | 7.67 | BLQ | 48.1 | 27.9 | |
| 0.50 | 30.0 | 9.28 | 728 | 256 | 409 |
| 1.00 | 960 | 101 | 1570 | 877 | 738 |
| 2.00 | 1540 | 836 | 2150 | 1510 | 658 |
| 4.00 | 1370 | 1080 | 1840 | 1430 | 384 |
| 6.00 | 1160 | 915 | 1540 | 1210 | 315 |
| 8.00 | 1050 | 809 | 1390 | 1080 | 292 |
| 12.0 | 854 | 693 | 1200 | 916 | 259 |
| 24.0 | 502 | 377 | 904 | 594 | 275 |
| 48.0 | 228 | 90.7 | 367 | 229 | 138 |
| 72.0 | 122 | 37.8 | 88.1 | 82.6 | 42.4 |
| 96.0 | 63.0 | 11.7 | 32.0 | 35.6 | 25.8 |
| 120 | 30.5 | 3.68 | 10.6 | 14.9 | 13.9 |
| 144 | 17.6 | 1.58 | 3.07 | 7.42 | 8.85 |
| 168 | 10.5 | BLQ | 1.75 | 6.13 | |

### Example 2. Encequidar Increases Oral Bioavailability and Decreases Intestinal Secretion Across Non-Clinical Species

Oral bioavailability of novel chemical entities can be limited by poor solubility, permeability, metabolic stability or transporter-mediated efflux. As one of the important factors, transporter-mediated efflux can greatly impact oral bioavailability (F %) and make achieving sufficient exposures a challenge (see e.g., Chan et al, European Journal of Pharmaceutical Sciences 2004, 21 (1), 25-51). Understanding the extent of transporter-mediated efflux in preclinical species such as rat, dog, and monkey can help with rational drug design to eliminate P-gp substrate affinity. For this reason, elacridar has been widely used as an efflux transporter inhibitor to help boost % F in preclinical species but suffers from poor formulatability (see e.g., Sawicki et al, Drug Delivery and Translational Research 2017, 7 (1), 125-131). Additionally, elacridar's systemic absorption can lead to drug-drug interactions (DDI) by inhibiting P-gp and BCRP at the bile-canalicular junction.

Recently, the ability of encequidar to inhibit P-gp was reported (see e.g., Chu et al, Pharm. Res. 2023; DOI: 10.1007/s11095-023-03563-4; and Smolinski et al, Journal of Medicinal Chemistry 2021, 64 (7), 3677-3693). Encequidar is a gut-restricted efflux transporter inhibitor that has been tested in rats and dogs with paclitaxel as a substrate (see e.g., Chu et al, Pharm. Res. 2023; DOI: 10.1007/s11095-023-03563-4). Encequidar is a useful tool to understand the influence of P-gp on % F limitations. Additionally, due to its gut-restricted properties, encequidar is a valuable tool for assessing the intestinal excretion clearance of molecules, a process mediated by efflux transporters such as P-gp and BCRP in the intestinal lumen. Because encequidar is not systemically absorbed, it selectively inhibits efflux transporters at the enterocyte level without affecting the bile-canalicular junction. This distinction enables encequidar to differentiate between intestinal secretion and biliary elimination, both influenced by systemic P-gp-mediated efflux.

This example describes the inhibition of encequidar in vitro against common transporters and cytochrome P450 enzymes to confirm its selectivity for human P-gp. In vivo experiments were then conducted in rats, dogs, and monkeys to demonstrate its impact on % F enhancement for P-gp substrates. To further illustrate encequidar's utility in distinguishing between intestinal excretion and biliary or hepatic metabolism-mediated clearances, IV studies on P-gp substrates known for intestinal excretion were conducted in rats and dogs.

### Formulation for In Vivo Assessment

The efflux probe substrate (test article) paclitaxel was formulated in 5% ethanol, 5% Kolliphor RH40, 20% PEG300, and 70% of 20% Kleptose in saline; digoxin was formulated in 5% NMP, 25% PEG300, and 70% of 20% Kleptose in saline; oral talinolol was formulated in 2% DMSO, 10% polyethylene glycol 300, and 88% sulfobutylether-β-cyclodextrin; IV talinolol and apixaban were co-formulated in 0.2% sodium phosphate, 30% 2-hydroxypropyl-β-cyclodextrin, and 69.8% water for injection. The efflux inhibitor encequidar was formulated in two forms-solution and suspension form: solution form was prepared in 5% NMP, 10% Kolliphor HS-15, 30% polyethylene glycol 300, and 55% water while the suspension form was prepared in 0.5% carboxymethylcellulose and 99.5% water. Elacridar HCl salt was formulated as a solution in 10% NMP, 20% PEG300, and 70% water.

### In Vivo Assessment in Sprague Dawley Rats

All animals were fasted overnight and had food returned at approximately 4 hour postdose. The inhibitor co-administered arm had a single dose of either encequidar (5 mg/kg, solution or suspension) or elacridar (10 mg/kg) 30 minutes prior to the test article. The no-inhibitor group was used as the control. Each group consisted of three male SD rats. The test article was either administered IV via lateral tail vein or PO via oral gavage. Whole blood was sampled up to 24 hours postdose following the test article administration and was treated with K2EDTA, after which was centrifuged to obtain plasma. Feces was collected only for the IV group, and at intervals of 0 to 4, 4 to 8, and 8 to 24 hours postdose.

### In Vivo Assessment in Beagle Dogs and Cynomolgus Monkeys

Both IV and PO studies were carried out in beagle dog. Talinolol and apixaban were selected as the efflux probe substrates for IV administration at 0.33 mg/kg dose, and paclitaxel was selected for oral administration at 5 mg/kg dose. All animals were fasted overnight and had food returned at approximately 4 hour postdose. The test group was administered 5 mg/kg encequidar (solution) 30-min prior to the administration of efflux probe substrate, and no inhibitor group was used as the control. Each group consisted of three animals. For the IV group, in addition to plasma, feces were also collected up to 72 hours. The same study design was used for monkey in vivo assessment, except that only oral administration of talinolol at 1 mg/kg dose was conducted.

### Bioanalytical Method

200 μL acetonitrile (ACN) containing internal standard was added to a 50 μL aliquot of each plasma and diluted feces homogenate (homogenized using 50% ACN (3 mL/mg feces)), as well as matrix blanks containing reference standards. The precipitated proteins were removed by centrifugation and 100 μL of supernatant was transferred into a clean 96 deep-well plate and mixed with 200 μL of water. An aliquot of 5 μL was injected into a Thermo Fisher Vanquish LC coupled with an Exploris 240 HRMS system. The chromatographic separation was carried out on a Thermo Scientific Hypersil GOLD C18 column (50× 2.1 mm, 1.9 μm particle size). The mobile phase consisted of 0.1% formic acid in water as solvent A and 0.1% formic acid in acetonitrile as solvent B. At 1 mL/min flow rate, the elution program started at 20% B and held for 0.2 min; then linearly ramped to 95% B at 1.1 min and held for 0.2 min. Finally the gradient was re-equilibrated to 20% B for another 0.2 min. Full mass analysis was performed under H-ESI positive mode using the exact mass of each analyte at 30,000 resolution with 5 ppm mass tolerance. The mass spectrometry parameters were configured as 3500 V ion spray voltage, sheath gas 60, auxiliary gas 15, and sweep gas 2 (all arbitrary unit), 350°C ion transfer tube temperature, and 400°C vaporizer temperature. The LCMS raw data were processed using TraceFinder 5.1 and the noncompartment analysis was carried out using Dotmatics Ver 5.5-100976-s [2021 May 28].

In vitro Transporter Inhibition Assay Overexpressing OATP 1B1 and 1B3 HEK 293 cells were seeded onto CellCoat Poly-D-Lysine coated 96-well black cell culture plates with clear bottoms at a density of 70,000 cells/well for WT and OATP1B1, or 100,000 cells/well for OATP1B3, respectively, and cultured to confluency overnight. All test compounds and reference inhibitors were serially diluted in DMSO to create spiking solutions at concentrations of 250-fold of final assay concentrations (final DMSO content 0.4%). Cells were preincubated with HBSS buffer containing test compounds for 15 minutes. After removing HBSS buffer, a 100 μL dose solution containing test article or reference inhibitor with probe substrate was added to the HEK293-OATP1B1, HEK-293-OATP1B3, and its associated WT cell plate, respectively. The co-incubation period of test compounds with the corresponding probe substrate for all plates was 5 minutes. Cells were washed three times with ice cold HBSS buffer and immediately extracted with 200 μL methanol:water (70:30; v: v) containing internal standard labetalol (30 nM). The 150 μL supernatants were evaporated to dryness on injection plates and reconstituted in 200 μL 80% water containing 20% acetonitrile and 0.1% formic acid for analysis by LC-MS/MS.

The transporter inhibition assay for human organic cation transporter (OCT) 2, organic anion transporter (OAT) 1 and 3, and multidrug and toxin extrusion protein (MATE) 1 and 2K, and breast cancer resistant protein (BCRP), and P-glycoprotein (P-gp) followed protocols from SOLVO Biotechnology (Budapest, Hungary). In brief, the in vitro test system was a polarized monolayer of MDCK-II cells grown on permeable supports. The MDCK-II cells were treated to express the transporter of interest or treated with a control vector. Unless stated otherwise, a stock solution was prepared with the test article in DMSO. All final assay solutions of probe substrate, reference inhibitor or test article contained 0.5% (v/v) DMSO. For OAT1, OAT3, OCT2, MATE1, and MATE2K, the pre-incubation time with inhibitors were 15 min and the uptake co-incubation time with probe substrate and inhibitors was 5 min. At the end of incubation, aspirated the uptake buffer and washed apical and basolateral side of the permeable support 4 times and added 60 μL cell extraction solution to the 96-well plate wells. After 15 min agitation on an orbital shaker at 120 rpm, collected 30 μL samples of the extract and added 200 μL scintillation fluid to extracted samples. The samples were counted on a 1450 Microbeta (Perkin-Elmer). For P-gp, the co-incubation time with probe substrate and inhibitors was 120 min; the co-incubation time with probe substrate and inhibitors was 60 min for BCRP. After incubation, aliquots (100 μL) were taken from the receptor chambers to determine the amount of translocated prazosin or digoxin, samples were analyzed by scintillation counting. The donor chambers were sampled before and after incubation to determine the initial concentration (CO) and recovery (R) of the probe substrate. Dose recovery samples were also taken from the helper plate to measure the actual initial concentration in the assay, the samples were quantified by LC-MS/MS.

### Results

### In Vitro Inhibition of Encequidar and Elacridar in HEK 293 or MDCK II Cells

As illustrated in Table 5, elacridar exhibited low IC₅₀ values (below 10 μM) for several transporters, including OATP1B1, OCT2, MATE1, MATE2K, P-gP, indicating its broad in vitro inhibition profile. Conversely, encequidar showed no inhibition of OATP1B1, OATP1B3, OAT3, or BCRP up to the highest tested concentration (100 μM). However, encequidar did display potent inhibition of OCT2, MATE1, MATE2K, and P-gp, with IC₅₀ values in the single-digit micromolar range, and weak inhibition of OAT1 (IC50=37 μM). This in vitro inhibition profile suggests that, unlike elacridar, encequidar preferentially inhibits P-gp without significantly affecting BCRP, highlighting its utility as selective P-gp inhibitor.

Encequidar is not a substrate for the transporters OATP1B1 and OATP1B3. Additionally, it exhibited no inhibitory effect on any tested cytochrome P450 enzymes (CYP1A2, 2B6, 2C8, 2C9, 2C19, 2D6, 3A4), with IC50 values exceeding 25 μM. Encequidar also did not act as a mechanism-based inhibitor of CYP3A4, as indicated by IC50 values greater than 25 μM in the presence and absence of NADPH. Encequidar demonstrated no inhibition of UGT1A1 (IC₅₀>20 μM), further confirming its limited impact on major metabolic enzymes.

Permeability assessments showed that encequidar was undetectable in the basolateral compartments of both Caco-2 and MDCK systems, indicating low passive permeability. This low permeability supports its classification as a gut-restricted inhibitor, allowing selective inhibition of intestinal P-gp without systemic inhibitory effects. This property is advantageous for differentiating intestinal secretion from biliary elimination pathways, making encequidar a useful tool for investigating transporter-mediated intestinal secretion.

TABLE 5
Encequidar and elacridar in vitro transporter inhibition.

| | |
|---|---|
| Probe | IC₅₀ (μM) |

| Transporter | substrate | Elacridar | Encequidar |
|---|---|---|---|
| OATP1B1 | Pravastatin | 3.2 | NIO up to |
| | | | 100 μM |
| OATP1B3 | Pravastatin | 44 | NIO up to |
| | | | 100 μM |
| OAT1 | Tenofovir | 27 | 37 |
| OAT3 | MTX | 55 | NIO up to |
| | | | 100 μM |
| OCT2 | Metformin | 0.62 | 0.25 |
| MATE1 | Metformin | 0.75 | 0.58 |
| MATE2K | Metformin | 0.17 | 0.13 |
| P-gp | Digoxin | 0.19 | 5.7 |
| BCRP | Prazosin | 6.8 | NIO up to |
| | | | 100 μM |

NIO: no inhibition observed;
MTX: methotrexate.

### Pharmacokinetics of Encequidar in Non-Clinical Species

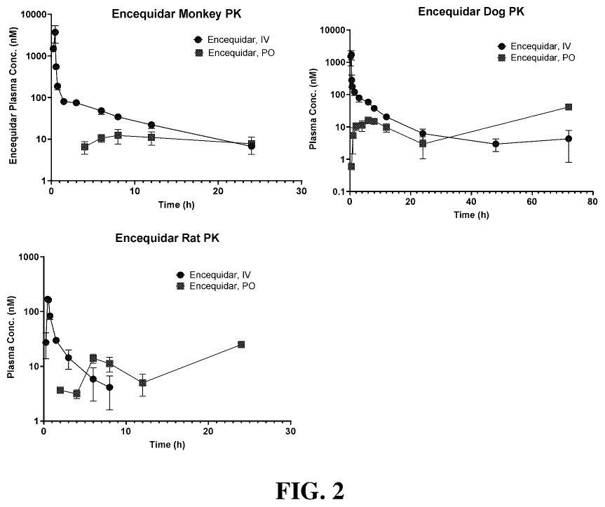

The PK profile of encequidar was investigated in rats, dogs, and monkeys, with the plasma PK profiles shown in FIG. 2 and summarized PK parameters in Table 6A-6B. Encequidar exhibited moderate to high plasma clearance across species, with hepatic extraction ratio (ER) ranging from 0.48 to >1, indicating substantial systemic elimination. Clearance was highest in rats, exceeding hepatic blood flow, likely due to the observed instability of encequidar in rat plasma (t_{1/2}=76 min), suggesting significant non-hepatic elimination pathways in this species.

The volume of distribution at steady state (Vₛₛ) was high across species, ranging from 3.2 L/kg in cynos to 15.4 L/kg in rats, indicative of extensive tissue distribution. The terminal half-life (t_{1/2}) varied markedly, from 2.1 hours in rats to 22.8 hours in dogs, highlighting species-specific differences in encequidar disposition. Encequidar demonstrated a biphasic PK profile characterized by rapid distribution followed by a slower elimination phase.

The oral bioavailability (F %) of encequidar was low in all species (<10%), consistent with its low passive permeability observed in Caco-2 and MDCK transwell systems. This limited oral absorption supports encequidar's utility as a gut-restricted P-gp inhibitor without significant systemic exposure, allowing selective inhibition of intestinal efflux without off-target effects.

In vitro stability assessment indicated that encequidar was stable in human, beagle dog, cynomolgus, and rhesus monkey plasma but degraded rapidly in rat plasma. Plasma protein binding was extensive in humans, cynomolgus monkeys, and rats, with a free fraction of less than 0.1% in plasma and human liver microsomes. Despite high plasma protein binding and low to moderate predicted hepatocytes extraction ratios (15-44% of hepatic blood flow across species), encequidar displayed moderate to high in vivo clearance. The elevated clearance in rats, exceeding hepatic blood flow, aligns with encequidar's plasma instability in this species.

In Tables 6A-6B, for IV administration, parameters include clearance (CL), predicted hepatic clearance (Pred Hep CL), volume of distribution at steady state (Vₛₛ), terminal half-life (t_{1/2}), and mean residence time (MRT). For PO administration, parameters include maximum plasma concentration (Cₘₐₓ), time to reach maximum plasma concentration (Tₘₐₓ), and oral bioavailability (% F).

TABLE 6A
Encequidar IV PK parameters

| | Rat | Dog | Monkey |
|---|---|---|---|
| CL, L/h/kg | 7.88 ± 1.54 (>1) | 0.869 ± 0.242 (0.54) | 0.781 ± 0.126 (0.49) |
| (ER) | | | |
| Pred Hep CL, | 1.76 | 0.72 | 0.25 |
| L/h/kg | | | |
| Vₛₛ, L/kg | 15.4 ± 1.99 | 7.73 ± 1.22 | 3.19 ± 0.749 |
| t_{1/2}, h | 2.09 ± 0.46 | 22.8 | 6.94 ± 0.748 |
| MRT, h | 2.04 ± 0.639 | 9.14 ± 1.62 | 4.05 ± 0.372 |

TABLE 6B
Encequidar PO PK parameters

| | Rat | Dog | Monkey |
|---|---|---|---|
| Cₘₐₓ, nM | 17.9 ± 6.48 | 16.9 ± 2.11 | 12.5 ± 4.38 |
| Tₘₐₓ, h | 12.7 ± 9.87 | 6.67 ± 1.15 | 7.33 ± 1.15 |
| % F | 8.23 ± 4 | 1.44 ± 0.225 | 1.03 ± 0.534 |

### Encequidar Boosts Oral Exposures of P-Gp Substrates Across Species

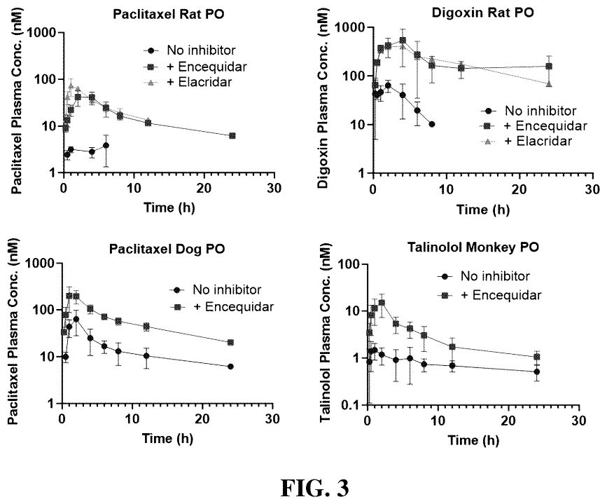

The pharmacokinetics of orally administered digoxin and paclitaxel in rats, with or without efflux inhibitors, are summarized in Table 7 and FIG. 3. Co-administration of encequidar led to a 10-fold increase in Cₘₐₓ and 20-fold increase in AUCₗₐₛₜ for digoxin, while elacridar produced similar effects with a 7-fold and 18-fold increase, respectively. For paclitaxel, encequidar co-administration resulted in a 13-fold increase in Cₘₐₓ and a 20-fold increase in AUCₗₐₛₜ, with elacridar showing comparable effects. Thus, both encequidar and elacridar markedly enhanced the oral exposures of digoxin and paclitaxel in rats.

Further experiments tested encequidar in a suspension formulation with paclitaxel to assess formulation impact on efflux inhibition. Results showed that the suspension formulation of encequidar achieved similar exposure increases as the solution, confirming that the observed inhibition is intrinsic to encequidar itself and not formulation-dependent.

In dogs, oral co-administration of encequidar with paclitaxel increased AUCₗₐₛₜ approximately 5-fold and Cₘₐₓ 4-fold, demonstrating effective P-gp inhibition in the gastrointestinal tract. Similarly, co-administration with talinolol in monkeys increased AUCₗₐₛₜ by 5-fold and Cₘₐₓ by 10-fold, further indicating potent P-gp inhibition by encequidar in primates.

The PK profiles show that the elimination phase of the substrates in dogs and monkeys was unaffected by encequidar, suggesting the increased oral exposure is primarily due to enhanced bioavailability rather than altered systemic clearance. Furthermore, as indicated in the encequidar PK section, plasma concentrations of encequidar remained below 10 nM post-5 mg/kg administration in rats, dogs, and monkeys, minimizing the likelihood of systemic inhibition of drug-metabolizing enzymes or other transporters.

In Table 7, “NC” indicates values that could not be calculated due to the absence of a clear terminal phase, and “NA” indicates parameters not applicable due to lack of fecal sample collection.

TABLE 7
Pharmacokinetic parameters of digoxin, paclitaxel, and talinolol in rats, dogs,
and monkeys following oral (PO) administration with or without co-administration
of the P-gp inhibitors encequidar (5 mg/kg PO) or elacridar (10 mg/kg PO)

| | Substrate, | Inhibitor, | AUCₗₐₛₜ | Cₘₐₓ, | Tₘₐₓ, |
|---|---|---|---|---|---|
| Species | Dose | Dose | nM*h | nM | nM |
| Rat | Digoxin, | None | 258 ± 135 | 64 ± 18 | 1.4 ± 1.0 |
| | 5 mg/kg | Encequidar, | 5170 ± 1280 | 623 ± 331 | 3.3 ± 1.2 |
| | | 5 mg/kg | | | |
| | | Elacridar, | 4720 ± 963 | 447 ± 58 | 3.3 ± 1.2 |
| | | 10 mg/kg | | | |
| Rat | Paclitaxel, | None | 15.3 ± 4.82 | 4.02 ± 1.48 | 2.7 ± 2.9 |
| | 5 mg/kg | Encequidar, | 361 ± 89 | 52.4 ± 2.84 | 2.7 ± 1.2 |
| | | 5 mg/kg | | | |
| | | Elacridar, | 358 ± 87 | 78.9 ± 24.5 | 1.3 ± 0.6 |
| | | 10 mg/kg | | | |
| Dog | Paclitaxel, | None | 300 ± 211 | 62.7 ± 34.8 | 2 |
| | 5 mg/kg | Encequidar, | 1470 ± 276 | 245 ± 68.4 | 1.67 ± 0.58 |
| | | 5 mg/kg | | | |
| Monkey | Talinolol, | None | 18.3 ± 5.3 | 1.56 ± 0.58 | 0.67 ± 0.29 |
| | 1 mg/kg | Encequidar, 5 | 83.7 ± 38.6 | 15.2 ± 7.92 | 2 ± 0 |
| | | mg/kg | | | |

### Encequidar can Inhibit Intestinal Secretion in Rat and Dog

Paclitaxel was dosed IV (1 mpk) in the absence and presence of 5 mpk PO encequidar or 10 mpk elacridar and the amount of parent was quantified in the feces. Encequidar and elacridar reduced the amount of parent in the feces from 20% to less than 1% thereby proving that encequidar and elacridar can inhibit Pgp in the enterocytes thereby stopping intestinal secretion. Correspondingly, the IV clearance of paclitaxel went up by 18% in the presence of the P-gp inhibitors.

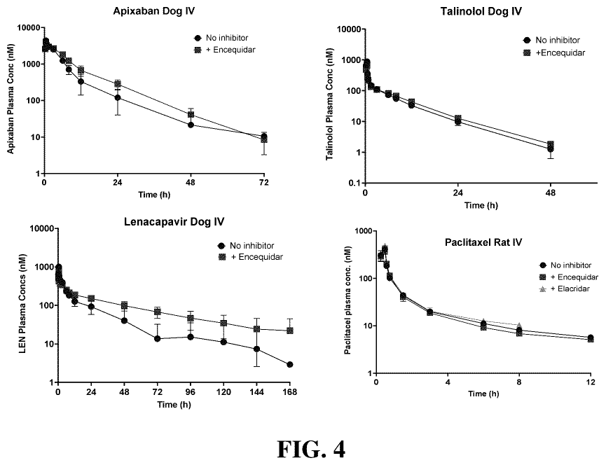

Encequidar is also capable of inhibiting intestinal secretion clearance and this was tested in dogs with apixaban, lenacapavir, and talinolol as compounds that undergo intestinal secretion. As shown in FIG. 4 and Table 8, following apixaban IV, approximately 31% was recovered in feces in unchanged form. This excretion in feces was reduced to 11% when co-administered with encequidar with a corresponding reduction in CL from 0.33 to 0.23 L/kg/h. Similarly, excretion of talinolol in feces was reduced from 10% to less than 1% with encequidar co-administration with a corresponding reduction in CL from 0.54 to 0.51 L/kg/h. With lenacapavir, the excretion in feces was reduced from 32 to 19% with a corresponding reduction in CL from 0.051 to 0.023 L/kg/h. Hence, in dogs, encequidar was capable of significantly reducing the extent of intestinal secretion. The parent found in feces upon coadministration of encequidar could be via biliary elimination, and hence encequidar is a useful tool to quantitatively estimate both intestinal secretion clearance and biliary clearance.

TABLE 8
Pharmacokinetic parameters of apixaban, talinolol, lenacapavir, and paclitaxel
in beagle dogs and Sprague-Dawley rats following IV administration, with or
without co-administration of the P-gp inhibitor encequidar (5 mg/kg PO)

| | | | | | | Excretion |
|---|---|---|---|---|---|---|
| Species | Substrate | Inhibitor | t_{1/2} (h) | CL (L/hr/kg) | Vₛₛ (L/kg) | in feces (%) |
| Dog | Apixaban | None | 6.9 ± 3.4 | 0.033 ± 0.008 | 0.21 ± 0.03 | 31 ± 10 |
| | | Encequidar | 10.1 ± 1.9 | 0.023 ± 0.004 | 0.22 ± 0.02 | 11 ± 3 |
| Dog | Talinolol | None | 7.6 ± 0.8 | 0.54 ± 0.06 | 3.88 ± 0.25 | 10 ± 3 |
| | | Encequidar | 7.6 ± 0.3 | 0.51 ± 0.05 | 4.43 ± 0.36 | 0.7 ± 0.2 |
| Dog | Lenacapavir | None | 15.9 ± 10.1 | 0.051 ± 0.019 | 0.90 ± 0.12 | 31.5 ± 9.0 |
| | | Encequidar | 45.1 ± 20.6 | 0.023 ± 0.008 | 1.34 ± 0.20 | 18.8 ± 4.1 |
| Rat | Paclitaxel | None | 4.0 ± 1.0 | 3.41 ± 0.81 | 6.5 ± 2.0 | 19.8 ± 6.8 |
| | | Encequidar | 6.5 ± 1.0 | 2.78 ± 0.12 | 12.4 ± 2.9 | 0.9 ± 0.7 |
| | | Elacridar | 5.2 ± 1.6 | 2.82 ± 0.47 | 7.5 ± 4.0 | 0.5 ± 0.3 |

### SUMMARY

### Encequidar is a Selective Human P-Gp Inhibitor

To understand the potency and selectivity of elacridar and encequidar, in vitro IC₅₀ of several common human efflux and uptake transporters using cells or membrane vesicles overexpressing these transporters were analyzed. Elacridar was found to potently inhibits multiple transporters, including OATP1B1, OCT2, MATE1, MATE2K, P-gp and BCRP with IC₅₀ values below 10 μM. Additionally, elacridar demonstrated moderate inhibition of OATP1B3, OAT1, and OAT3 with IC₅₀ values ranging from 27 to 55 μM. In contrast, encequidar showed no measurable inhibition of OATP1B1, OATP1B3, OAT3, and BCRP, and only moderate inhibition towards OAT1 (IC₅₀=37 μM).

In pharmacokinetic studies conducted in rats, dogs and monkeys, oral bioavailability of encequidar was observed to be less than 10%, with Cₘₐₓ values below 20 nM. Given encequidar's high plasma protein binding (>99.9% across species), and without being bound by theory, the estimated free circulating concentrations post-oral administration are likely to be in single to double-digit picomolar range, suggesting minimal potential for systemic inhibition. Furthermore, encequidar does not inhibit major cytochrome P450 enzymes and is not a time-dependent inhibitor of CYP3A4.

The in vitro transporter and enzyme inhibition data, combined with the in vivo PK profile, indicate that encequidar's P-gp inhibition is restricted to the gastrointestinal tract. Its selectivity for human P-gp over human BCRP makes it a useful tool for investigating P-gp-mediated efflux mechanism.

### Encequidar Shows High Clearance and Low Oral Bioavailability

Encequidar was administered to rats, dogs, and monkeys, and exhibited moderate to high clearance across species. In cryopreserved hepatocyte assays, predicted clearance was low to moderate, yet in vivo clearance remained moderate to high, despite encequidar's very high plasma protein binding (>99.9% in human, rat, dog and monkey). This discrepancy suggests that factors beyond hepatic metabolism, such as plasma instability, contribute to encequidar's clearance, especially in rats, where clearance exceeded hepatic blood flow, aligning with its observed instability in rat plasma.

In dogs and monkeys, encequidar displayed a biphasic pharmacokinetic profile with a long terminal half-life ranging from 7 to 23 hours. Following oral dosing at 10 mg/kg in solution, encequidar exposures were very low with bioavailability ranging from 1% to 8% across species and Cₘₐₓ values consistently below 20 nM. These low oral exposures correlate well with the low permeability determined from MDCK and Caco-2 where the permeability values were below the detection limit of 0.1×10⁻⁶ cm/s. The Fₐ×F_{g} values (fraction absorbed and fraction escaping gut metabolism) for dog and monkey were 2 and 3% respectively, indicating that both poor permeability and metabolic instability contribute to encequidar's limited oral exposures. These findings underscore encequidar's gut-restricted nature, suggesting that any pharmacokinetic impact or DDIs with encequidar as the perpetrator would be limited to the GI tract with minimal systemic effects.

### Encequidar Boosts Oral Bioavailability of P-Gp Substrates Across Preclinical Species

Administration of 5 mg/kg encequidar in a solution formulation increased Cₘₐₓ and AUC values of P-gp substrates by 5- to 20-fold across rat, dog and monkey. This study represents the first comprehensive report of encequidar's ability to enhance P-gp substrate exposures across multiple preclinical species, highlighting its utility as a pharmacokinetic booster. Given encequidar's gut-restricted nature, this enhancement in oral bioavailability is due to efflux transporter inhibition at enterocyte level, suggesting that encequidar has sufficient permeability to access enterocytes and inhibit the efflux transporters, yet lacks the permeability and metabolic stability necessary for significant systemic exposure.

Furthermore, comparable bioavailability improvements for paclitaxel were observed with both suspension and solution formulations of encequidar, indicating formulation flexibility. This ease of formulation, combined with its selective enterocyte-level action, makes encequidar an attractive tool for investigating drug disposition mechanisms.

### Encequidar is Capable of Inhibiting Intestinal Secretion in Rat and Dog

When paclitaxel was administered intravenously in rats, co-administration of oral elacridar reduced the fecal excretion of paclitaxel from 20% to 1% of the administered dose. This suggests that approximately 20% of paclitaxel could be eliminated via biliary elimination or intestinal secretion, both of which are inhibited by elacridar. However, when a similar 20% reduction of fecal excretion was observed with the co-administration of encequidar, this indicates that paclitaxel undergoes intestinal secretion in rats, instead of biliary elimination. Thus, encequidar serves as a useful tool not only for quantifying the extent of intestinal secretion, but also for differentiating between intestinal and biliary elimination pathways.

Further investigations into the role of encequidar in inhibiting intestinal secretion were conducted in dogs, where apixaban, talinolol, and lenacapavir were co-administered as an IV cassette at 0.33 mg/kg each. For apixaban, fecal excretion decreased from 31 to 11% with encequidar, suggesting approximately 20% intestinal secretion and 11% biliary elimination in dogs. This decrease in fecal excretion was consistent with a 30% reduction in IV clearance. For talinolol, fecal excretion decreased from 10% to 1%, indicating around 9% intestinal secretion and negligible biliary elimination. Correspondingly, IV clearance decreased by 6%, aligning with the reduction in intestinal secretion. For lenacapavir, fecal excretion decreased from 32% to 19%, indicating 13% intestinal secretion and 19% biliary elimination. Lenacapavir's IV clearance reduced by 54%, which exceeded the reduction in intestinal secretion.

In summary, this study demonstrates that encequidar acts as a gut-restricted efflux inhibitor in rats, dogs, and monkeys. Oral co-administration of encequidar PO significantly enhances the oral bioavailability of the efflux substrates and reduces their intestinal excretion into feces, This gut-specific inhibitory effect on intestinal secretion makes encequidar a valuable tool for assessing the contribution of intestinal excretion in the overall disposition of drug candidates in preclinical models, without the need for bile duct cannulation (BDC) studies.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating or preventing a human immunodeficiency virus (HIV) infection in a patient in need thereof, comprising administering to the patient:
(a) a compound of Formula Ia: 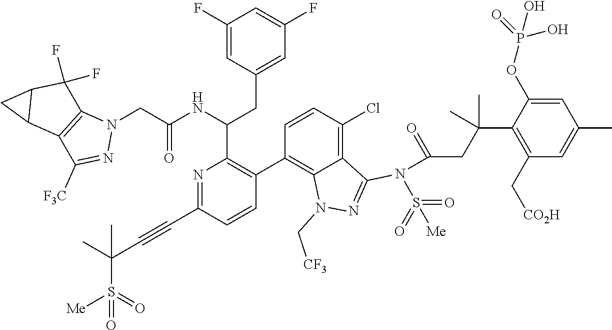 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]
or a pharmaceutically acceptable salt thereof; and
(b) a compound of Formula II: 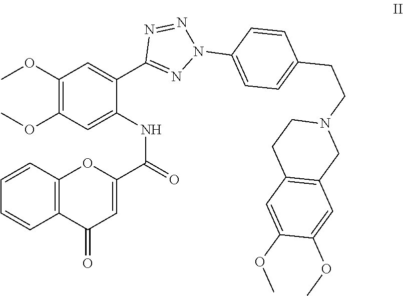 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]
or a pharmaceutically acceptable salt thereof.

2. A method of treating or preventing a human immunodeficiency virus (HIV) infection in a patient in need thereof, comprising administering to the patient:
(a) a pharmaceutical composition comprising a compound of Formula Ia: 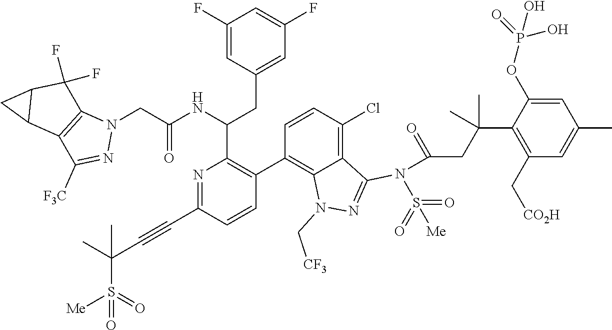 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]
or a pharmaceutically acceptable salt thereof, and one or more pharmaceutically acceptable excipients; and
(b) a pharmaceutical composition comprising a compound of Formula II: 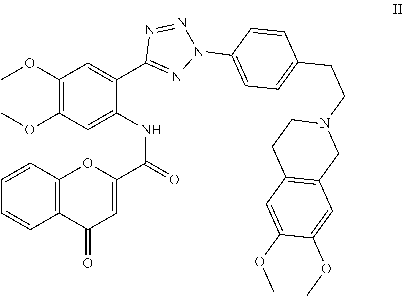 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]
or a pharmaceutically acceptable salt thereof, and one or more pharmaceutically acceptable excipients.

3. The method of claim 2, wherein the pharmaceutical composition comprising the compound of Formula Ia, or a pharmaceutically acceptable salt thereof, is a tablet.

4-8. (canceled)

9. The method of claim 3, wherein the tablet comprises;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

10-30. (canceled)

31. The method of claim 3, wherein the method comprises orally administering to the patient a tablet comprising:
about 5 mg to about 700 mg of the compound of Formula Ia, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

32. The method of claim 3, wherein the method comprises orally administering to the patient a tablet comprising:
about 175 mg to about 225 mg of the compound of Formula Ia, or a pharmaceutically acceptable salt thereof;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

33. The method of claim 3, wherein the method comprises orally administering to the patient a tablet comprising:
about 200 mg of the compound of Formula Ia, or a pharmaceutically acceptable salt thereof;
about 135 mg of mannitol;
about 137.5 mg of microcrystalline cellulose;
about 20 mg of crospovidone; and
about 7.5 mg of magnesium stearate.

34. The method of claim 3, wherein the tablet is administered to the patient once every seven days.

35-37. (canceled)

38. The method of claim 2, wherein the pharmaceutical composition comprising the compound of Formula II, or a pharmaceutically acceptable salt thereof, is a solution or a suspension suitable for oral administration.

39-57. (canceled)

58. The method of claim 38, wherein the suspension comprises about 2 mg/mL of the compound of Formula II, or a pharmaceutically acceptable salt thereof, about 0.5 w/w % carboxymethyl cellulose, and 99.5 w/w % water.

59. (canceled)

60. The method of claim 38, wherein the solution comprises about 2 mg/mL of the compound of Formula II, or a pharmaceutically acceptable salt thereof, about 10 w/w % Kolliphor HS-15, about 5 w/w % N-methylpyrrolidone, about 30 w/w % PEG 300, and about 55 w/w % water.

61. The method of claim 1, which is a method of preventing a human immunodeficiency virus (HIV) infection in the patient.

62. (canceled)

63. The method of claim 1, wherein the method comprises pre-exposure prophylaxis (PrEP).

64-66. (canceled)

67. The method of claim 1, wherein the compound of Formula Ia, or a pharmaceutically acceptable salt thereof, and the compound of Formula II, or a pharmaceutically acceptable salt thereof, are administered before exposure of the patient to the HIV.

68-75. (canceled)

76. The method of claim 1, which is a method of treating HIV.

77. The method of claim 1, wherein the method further comprises administering a therapeutically effective amount of one, two, three, or four additional therapeutic agents, or a pharmaceutically acceptable salt thereof.

78-80. (canceled)

81. The method of claim 1, wherein the compound of Formula Ia, or a pharmaceutically acceptable salt thereof, is a compound of Formula Ib: 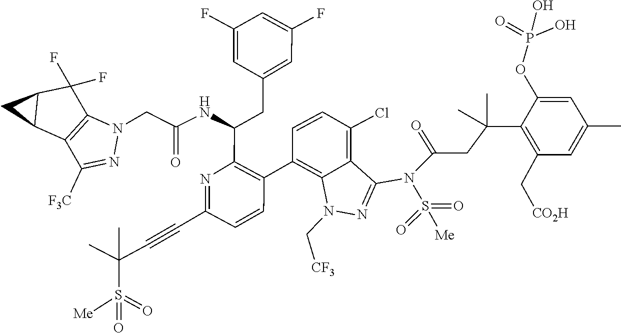 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]
or a pharmaceutically acceptable salt thereof.

82. The method of claim 1, wherein the compound of Formula Ia, or a pharmaceutically acceptable salt thereof, is a crystalline form of a compound of Formula Ib:

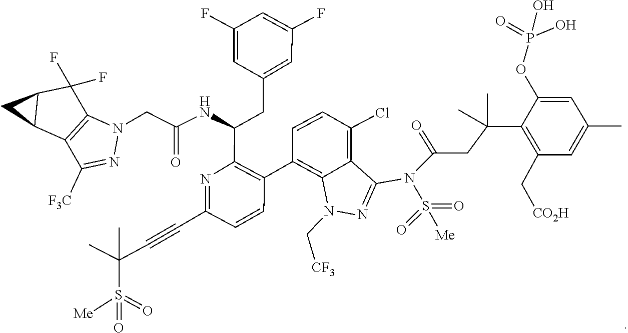 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

83. The method of claim 82, wherein the crystalline form of the compound of Formula Ib is crystalline Form I.

84. (canceled)

85. The method of claim 1, wherein the compound of Formula II is administered as a mesylate salt.

86. The method of claim 1, wherein the HIV is HIV-1.

87. The method of claim 1, wherein the patient is a human.

88-93. (canceled)
